SEQUENCE LISTING (85 sequences)

SEQ ID NO: 1 - DNA, 150 bp, Homo sapiens
cugaagucgcuugagaaagaaauugcuggccaagaugaggaccacuucauuacaguggacgcuguggguugcuucgagggugaugaagaagaggaagaggaugaugaggaugaagaagagaucgagguugaggaggaacucugcaagcag

SEQ ID NO: 2 - protein, 50 aa, Homo sapiens
LKSLEKEIAGQDEDHFITVDAVGCFEGDEEEEEDDEDEEEIEVEEELCKQ

SEQ ID NO: 3 - DNA, 126 bp, Homo sapiens
cugaagucgcuugagaaagaaauugcuggccaagaugaggaccacuucauuacaguggacgcuguggguugcuucgagggugaugaagaagaggaagaggaugaugaggaugaagaagagaucgag

SEQ ID NO: 4 - protein, 42 aa, Homo sapiens
LKSLEKEIAGQDEDHFITVDAVGCFEGDEEEEEDDEDEEEIE

SEQ ID NO: 5 - DNA, 69 bp, Homo sapiens
gugagguccagagauauauccagagaggaguggaagggcucggagaccuacagccccaauacugcauau

SEQ ID NO: 6 - protein, 23 aa, Homo sapiens
VRSRDISREEWKGSETYSPNTAY

SEQ ID NO: 7 - DNA, 50 bp, Homo sapiens
ugaugaggaugaagaagagaucgaggugagguccagagauauauccagag

SEQ ID NO: 8 - protein, 16 aa, Homo sapiens
DEDEEEIEVRSRDISR

SEQ ID NO: 9 - protein, 457 aa, Homo sapiens
MLQRALLLQQLQGLDQFAMPPATYDTAGLTMPTATLGNLRGYGMASPGLAAPSLTPPQLATPNLQQFFPQATRQSLLGPPPVGVPMNPSQFNLSGRNPQKQARTSSSTTPNRKDSSSQTMPVEDKSDPPEGSEEAAEPRMDTPEDQDLPPCPEDIAKEKRTPAPEPEPCEASELPAKRLRSSEEPTEKEPPGQLQVKAQPQARMTVPKQTQTPDLLPEALEAQVLPRFQPRVLQVQAQVQSQTQPRIPSTDTQVQPKLQKQAQTQTSPEHLVLQQKQVQPQLQQEAEPQKQVQPQVQPQAHSQGPRQVQLQQEAEPLKQVQPQVQPQAHSQPPRQVQLQLQKQVQTQTYPQVHTQAQPSVQPQEHPPAQVSVQPPEQTHEQPHTQPQVSLLAPEQTPVVVHVCGLEMPPDAVEAGGGMEKTLPEPVGTQVSMEEIQNESACGLDVGECENRAREMPG

SEQ ID NO: 10 - protein, 414 aa, Homo sapiens
MASPGLAAPSLTPPQLATPNLQQFFPQATRQSLLGPPPVGVPMNPSQFNLSGRNPQKQARTSSSTTPNRKDSSSQTMPVEDKSDPPEGSEEAAEPRMDTPEDQDLPPCPEDIAKEKRTPAPEPEPCEASELPAKRLRSSEEPTEKEPPGQLQVKAQPQARMTVPKQTQTPDLLPEALEAQVLPRFQPRVLQVQAQVQSQTQPRIPSTDTQVQPKLQKQAQTQTSPEHLVLQQKQVQPQLQQEAEPQKQVQPQVQPQAHSQGPRQVQLQQEAEPLKQVQPQVQPQAHSQPPRQVQLQLQKQVQTQTYPQVHTQAQPSVQPQEHPPAQVSVQPPEQTHEQPHTQPQVSLLAPEQTPVVVHVCGLEMPPDAVEAGGGMEKTLPEPVGTQVSMEEIQNESACGLDVGECENRAREMPG

SEQ ID NO: 11 - protein, 358 aa, Homo sapiens
MASPGLAAPSLTPPQLATPNLQQFFPQATRQSLLGPPPVGVPMNPSQFNLSGRNPQKQARTSSSTTPNRKDSSSQTMPVEDKSDPPEGSEEAAEPRMDTPEDQDLPPCPEDIAKEKRTPAPEPEPCEASELPAKRLRSSEEPTEKEPPGQLQVKAQPQARMTVPKQTQTPDLLPEALEAQVLPRFQPRVLQVQAQVQSQTQPRIPSTDTQVQPKLQKQAQTQTSPEHLVLQQKQVQPQLQQEAEPQKQVQPQVHTQAQPSVQPQEHPPAQVSVQPPEQTHEQPHTQPQVSLLAPEQTPVVVHVCGLEMPPDAVEAGGGMEKTLPEPVGTQVSMEEIQNESACGLDVGECENRAREMPG

SEQ ID NO: 12 - DNA, 1371 bp, Homo sapiens
augcugcagagagcuuugcuuuuacagcaguugcaaggacuggaccaguuugcaaugccaccagccacguaugacacugccggucucaccaugcccacagcaacacuggguaaccuccgaggcuauggcauggcauccccaggccucgcagcccccagccucacacccccacaacuggccacuccaaauuugcaacaguucuuuccccaggccacucgccaguccuugcugggaccuccuccuguugggguccccaugaacccuucccaguucaaccuuucaggacggaacccccagaaacaggcccggaccuccuccucuaccacccccaaucgaaaggauucuucuucucagacaaugccuguggaagacaagucagaccccccagaggggucugaggaagccgcagagccccggauggacacaccagaagaccaagauuuaccgcccugcccagaggacaucgccaaggaaaaacgcacuccagcaccugagccugagccuugugaggcguccgagcugccagcaaagagauugaggagcucagaagagcccacagagaaggaaccuccagggcaguuacaggugaaggcccagccgcaggcccggaugacaguaccgaaacagacacagacaccagaccugcugccugaggcccuggaagcccaagugcugccacgauuccagccacggguccugcagguccaggcccaggugcagucacagacucagccgcggauaccauccacagacacccaggugcagccaaagcugcagaagcaggcgcaaacacagaccucuccagagcacuuagugcugcaacagaagcaggugcagccacagcugcagcaggaggcagagccacagaagcaggugcagccacagguacagccacaggcacauucacagggcccaaggcaggugcagcugcagcaggaggcagagccgcugaagcaggugcagccacaggugcagccccaggcacauucacagcccccaaggcaggugcagcugcagcugcagaagcagguccagacacagacauauccacagguccacacacaggcacagccaagcguccagccacaggagcauccuccagcgcaggugucaguacagccaccagagcagacccaugagcagccucacacccagccgcaggugucguugcuggcuccagagcaaacaccaguugugguucaugucugcgggcuggagaugccaccugaugcaguagaagcugguggaggcauggaaaagaccuugccagagccugugggcacccaagucagcauggaagagauucagaaugagucggccuguggccuagaugugggagaaugugaaaacagagcgagagagaugccaggg

SEQ ID NO: 13 - DNA, 1263 bp, Homo sapiens
gguaaccuccgaggcuauggcauggcauccccaggccucgcagcccccagccucacacccccacaacuggccacuccaaauuugcaacaguucuuuccccaggccacucgccaguccuugcugggaccuccuccuguugggguccccaugaacccuucccaguucaaccuuucaggacggaacccccagaaacaggcccggaccuccuccucuaccacccccaaucgaaaggauucuucuucucagacaaugccuguggaagacaagucagaccccccagaggggucugaggaagccgcagagccccggauggacacaccagaagaccaagauuuaccgcccugcccagaggacaucgccaaggaaaaacgcacuccagcaccugagccugagccuugugaggcguccgagcugccagcaaagagauugaggagcucagaagagcccacagagaaggaaccuccagggcaguuacaggugaaggcccagccgcaggcccggaugacaguaccgaaacagacacagacaccagaccugcugccugaggcccuggaagcccaagugcugccacgauuccagccacggguccugcagguccaggcccaggugcagucacagacucagccgcggauaccauccacagacacccaggugcagccaaagcugcagaagcaggcgcaaacacagaccucuccagagcacuuagugcugcaacagaagcaggugcagccacagcugcagcaggaggcagagccacagaagcaggugcagccacagguacagccacaggcacauucacagggcccaaggcaggugcagcugcagcaggaggcagagccgcugaagcaggugcagccacaggugcagccccaggcacauucacagcccccaaggcaggugcagcugcagcugcagaagcagguccagacacagacauauccacagguccacacacaggcacagccaagcguccagccacaggagcauccuccagcgcaggugucaguacagccaccagagcagacccaugagcagccucacacccagccgcaggugucguugcuggcuccagagcaaacaccaguugugguucaugucugcgggcuggagaugccaccugaugcaguagaagcugguggaggcauggaaaagaccuugccagagccugugggcacccaagucagcauggaagagauucagaaugagucggccuguggccuagaugugggagaaugugaaaacagagcgagagagaugccaggg

SEQ ID NO: 14 - DNA, 1095 bp, Homo sapiens
gguaaccuccgaggcuauggcauggcauccccaggccucgcagcccccagccucacacccccacaacuggccacuccaaauuugcaacaguucuuuccccaggccacucgccaguccuugcugggaccuccuccuguugggguccccaugaacccuucccaguucaaccuuucaggacggaacccccagaaacaggcccggaccuccuccucuaccacccccaaucgaaaggauucuucuucucagacaaugccuguggaagacaagucagaccccccagaggggucugaggaagccgcagagccccggauggacacaccagaagaccaagauuuaccgcccugcccagaggacaucgccaaggaaaaacgcacuccagcaccugagccugagccuugugaggcguccgagcugccagcaaagagauugaggagcucagaagagcccacagagaaggaaccuccagggcaguuacaggugaaggcccagccgcaggcccggaugacaguaccgaaacagacacagacaccagaccugcugccugaggcccuggaagcccaagugcugccacgauuccagccacggguccugcagguccaggcccaggugcagucacagacucagccgcggauaccauccacagacacccaggugcagccaaagcugcagaagcaggcgcaaacacagaccucuccagagcacuuagugcugcaacagaagcaggugcagccacagcugcagcaggaggcagagccacagaagcaggugcagccacagguccacacacaggcacagccaagcguccagccacaggagcauccuccagcgcaggugucaguacagccaccagagcagacccaugagcagccucacacccagccgcaggugucguugcuggcuccagagcaaacaccaguugugguucaugucugcgggcuggagaugccaccugaugcaguagaagcugguggaggcauggaaaagaccuugccagagccugugggcacccaagucagcauggaagagauucagaaugagucggccuguggccuagaugugggagaaugugaaaacagagcgagagagaugccaggg

SEQ ID NO: 15 - protein, 399 aa, Homo sapiens
GMEKTLPEPVGTQVSMEEIQNESACGLDVGECENRAREMPGVWGAGGSLKVTILQSSDSRAFSTVPLTPVPRPSDSVSSTPAATSTPSKQALQFFCYICKASCSSQQEFQDHMSEPQHQQRLGEIQHMSQACLLSLLPVPRDVLETEDEEPPPRRWCNTCQLYYMGDLIQHRRTQDHKIAKQSLRPFCTVCNRYFKTPRKFVEHVKSQGHKDKAKELKSLEKEIAGQDEDHFITVDAVGCFEGDEEEEEDDEDEEEIEVEEELCKQVRSRDISREEWKGSETYSPNTAYGVDFLVPVMGYICRICHKFYHSNSGAQLSHCKSLGHFENLQKYKAAKNPSPTTRPVSRRCAINARNALTALFTSSGRPPSQPNTQDKTPSKVTARPSQPPLPRRSTRLKT

SEQ ID NO: 16 - protein, 227 aa, Homo sapiens
RTQDHKIAKQSLRPFCTVCNRYFKTPRKFVEHVKSQGHKDKAKELKSLEKEIAGQDEDHFITVDAVGCFEGDEEEEEDDEDEEEIEVEEELCKQVRSRDISREEWKGSETYSPNTAYGVDFLVPVMGYICRICHKFYHSNSGAQLSHCKSLGHFENLQKYKAAKNPSPTTRPVSRRCAINARNALTALFTSSGRPPSQPNTQDKTPSKVTARPSQPPLPRRSTRLKT

SEQ ID NO: 17 - protein, 208 aa, Homo sapiens
NRYFKTPRKFVEHVKSQGHKDKAKELKSLEKEIAGQDEDHFITVDAVGCFEGDEEEEEDDEDEEEIEVEEELCKQVRSRDISREEWKGSETYSPNTAYGVDFLVPVMGYICRICHKFYHSNSGAQLSHCKSLGHFENLQKYKAAKNPSPTTRPVSRRCAINARNALTALFTSSGRPPSQPNTQDKTPSKVTARPSQPPLPRRSTRLKT

SEQ ID NO: 18 - DNA, 1200 bp, Homo sapiens
ggcauggaaaagaccuugccagagccugugggcacccaagucagcauggaagagauucagaaugagucggccuguggccuagaugugggagaaugugaaaacagagcgagagagaugccagggguauggggcgccgggggcucccugaaggucaccauucugcagagcagugacagccgggccuuuagcacuguaccccugacaccugucccccgccccagugacuccgucuccuccaccccugcggcuaccagcacucccucuaagcaggcccuccaguucuucugcuacaucugcaaggccagcugcuccagccagcaggaguuccaggaccacaugucggagccucagcaccagcagcggcuaggggagauccagcacaugagccaagccugccuccugucccugcugcccgugccccgggacguccuggagacagaggaugaggagccuccaccaaggcgcuggugcaacaccugccagcucuacuacaugggggaccugauccaacaccgcaggacacaggaccacaagauugccaaacaauccuugcgacccuucugcaccguuugcaaccgcuacuucaaaaccccucgcaaguuuguggagcacgugaagucccaggggcauaaggacaaagccaaggagcugaagucgcuugagaaagaaauugcuggccaagaugaggaccacuucauuacaguggacgcuguggguugcuucgagggugaugaagaagaggaagaggaugaugaggaugaagaagagaucgagguugaggaggaacucugcaagcaggugagguccagagauauauccagagaggaguggaagggcucggagaccuacagccccaauacugcauaugguguggacuuccuggugcccgugaugggcuauaucugccgcaucugccacaaguucuaucacagcaacucaggggcacagcucucccacugcaagucccugggccacuuugagaaccugcagaaauacaaggcggccaagaaccccagccccaccacccgaccugugagccgccggugcgcaaucaacgcccggaacgcuuugacagcccuguucaccuccagcggccgcccacccucccagcccaacacccaggacaaaacacccagcaaggugacggcucgacccucccagcccccacuaccucggcgcucaacccgccucaaaaccuga

SEQ ID NO: 19 - DNA, 684 bp, Homo sapiens
aggacacaggaccacaagauugccaaacaauccuugcgacccuucugcaccguuugcaaccgcuacuucaaaaccccucgcaaguuuguggagcacgugaagucccaggggcauaaggacaaagccaaggagcugaagucgcuugagaaagaaauugcuggccaagaugaggaccacuucauuacaguggacgcuguggguugcuucgagggugaugaagaagaggaagaggaugaugaggaugaagaagagaucgagguugaggaggaacucugcaagcaggugagguccagagauauauccagagaggaguggaagggcucggagaccuacagccccaauacugcauaugguguggacuuccuggugcccgugaugggcuauaucugccgcaucugccacaaguucuaucacagcaacucaggggcacagcucucccacugcaagucccugggccacuuugagaaccugcagaaauacaaggcggccaagaaccccagccccaccacccgaccugugagccgccggugcgcaaucaacgcccggaacgcuuugacagcccuguucaccuccagcggccgcccacccucccagcccaacacccaggacaaaacacccagcaaggugacggcucgacccucccagcccccacuaccucggcgcucaacccgccucaaaaccuga

SEQ ID NO: 20 - DNA, 629 bp, Homo sapiens
gcaaccgcuacuucaaaaccccucgcaaguuuguggagcacgugaagucccaggggcauaaggacaaagccaaggagcugaagucgcuugagaaagaaauugcuggccaagaugaggaccacuucauuacaguggacgcuguggguugcuucgagggugaugaagaagaggaagaggaugaugaggaugaagaagagaucgagguugaggaggaacucugcaagcaggugagguccagagauauauccagagaggaguggaagggcucggagaccuacagccccaauacugcauaugguguggacuuccuggugcccgugaugggcuauaucugccgcaucugccacaaguucuaucacagcaacucaggggcacagcucucccacugcaagucccugggccacuuugagaaccugcagaaauacaaggcggccaagaaccccagccccaccacccgaccugugagccgccggugcgcaaucaacgcccggaacgcuuugacagcccuguucaccuccagcggccgcccacccucccagcccaacacccaggacaaaacacccagcaaggugacggcucgacccucccagcccccacuaccucggcgcucaacccgccucaaaaccuga

SEQ ID NO: 21 - protein, 73 aa, Homo sapiens
LKSLEKEIAGQDEDHFITVDAVGCFEGDEEEEEDDEDEEEIEVEEELCKQVRSRDISREEWKGSETYSPNTAY

SEQ ID NO: 22 - protein, 65 aa, Homo sapiens
LKSLEKEIAGQDEDHFITVDAVGCFEGDEEEEEDDEDEEEIEVRSRDISREEWKGSETYSPNTAY

SEQ ID NO: 23 - protein, 24 aa, Homo sapiens
DEDEEEIEVEEELCKQVRSRDISR

SEQ ID NO: 24 - DNA, 21 bp, Homo sapiens
aagaagagaucgaggugaggu

SEQ ID NO: 25 - DNA, 21 bp, Homo sapiens
aagagaucgaggugaggucca

SEQ ID NO: 26 - DNA, 21 bp, Homo sapiens
agaagagaucgaggugagguc

SEQ ID NO: 27 - DNA, 21 bp, Homo sapiens
gaagagaucgaggugaggucc

SEQ ID NO: 28 - DNA, 21 bp, Homo sapiens
agagaucgaggugagguccag

SEQ ID NO: 29 - protein, 9 aa, Human immunodeficiency virus
RKKRRQRRR

SEQ ID NO: 30 - protein, 70 aa, Homo sapiens
FFMEELNTYRQKQGVVLKYQELPNSGPPHDRRFTFQVIIDGREFPEGEGRSKKEAKNAAAKLAVEILNKE

SEQ ID NO: 31 - DNA, 20 bp, Artificial Sequence
  Nucleic acid product amplified using PCR
tggacctcacctcgatctct

SEQ ID NO: 32 - DNA, 40 bp, Artificial Sequence
  Nucleic acid product amplified using PCR
gatatatctctggacctcacctcgatctcttcttcatcct

SEQ ID NO: 33 - DNA, 29 bp, Artificial Sequence
  Nucleic acid product amplified using PCR
gaagaagagatcgaggtgaggtccagaga

SEQ ID NO: 34 - DNA, 20 bp, Artificial Sequence
  Primer
gaagagatcgaggtgaggtc

SEQ ID NO: 35 - DNA, 26 bp, Artificial Sequence
  Oligonucleotide probe
tggacctcacctcgatctcttcttca

SEQ ID NO: 36 - protein, 6 aa, Homo sapiens
DEEEIE

SEQ ID NO: 37 - protein, 7 aa, Homo sapiens
VRSRDIS

SEQ ID NO: 38 - protein, 21 aa, Homo sapiens
DEEEIEVEEELCKQVRSRDIS

SEQ ID NO: 39 - protein, 34 aa, Homo sapiens
EGDEEEEEDDEDEEEIEVRSRDISREEWKGSETY

SEQ ID NO: 40 - protein, 42 aa, Homo sapiens
EGDEEEEEDDEDEEEIEVEEELCKQVRSRDISREEWKGSETY

SEQ ID NO: 41 - protein, 30 aa, Homo sapiens
DEEEEEDDEDEEEIEVRSRDISREEWKGSE

SEQ ID NO: 42 - protein, 38 aa, Homo sapiens
DEEEEEDDEDEEEIEVEEELCKQVRSRDISREEWKGSE

SEQ ID NO: 43 - protein, 26 aa, Homo sapiens
EEEEDDEDEEEIEVRSRDISREEWKG

SEQ ID NO: 44 - protein, 34 aa, Homo sapiens
EEEEDDEDEEEIEVEEELCKQVRSRDISREEWKG

SEQ ID NO: 45 - protein, 22 aa, Homo sapiens
EEDDEDEEEIEVRSRDISREEW

SEQ ID NO: 46 - protein, 30 aa, Homo sapiens
EEDDEDEEEIEVEEELCKQVRSRDISREEW

SEQ ID NO: 47 - protein, 18 aa, Homo sapiens
DDEDEEEIEVRSRDISRE

SEQ ID NO: 48 - protein, 26 aa, Homo sapiens
DDEDEEEIEVEEELCKQVRSRDISRE

SEQ ID NO: 49 - protein, 13 aa, Homo sapiens
DEEEIEVRSRDIS

SEQ ID NO: 50 - protein, 14 aa, Homo sapiens
EDEEEIEVRSRDIS

SEQ ID NO: 51 - protein, 22 aa, Homo sapiens
EDEEEIEVEEELCKQVRSRDIS

SEQ ID NO: 52 - protein, 12 aa, Homo sapiens
DEEEIEVRSRDI

SEQ ID NO: 53 - protein, 20 aa, Homo sapiens
DEEEIEVEEELCKQVRSRDI

SEQ ID NO: 54 - protein, 7 aa, Homo sapiens
EIEVRSR

SEQ ID NO: 55 - protein, 15 aa, Homo sapiens
EIEVEEELCKQVRSR

SEQ ID NO: 56 - DNA, 29 bp, Homo sapiens
gaagaagagaucgaggugagguccagaga

SEQ ID NO: 57 - DNA, 20 bp, Artificial Sequence
  Primer
cacaactggccactccaaat

SEQ ID NO: 58 - DNA, 19 bp, Artificial Sequence
  Primer
cctctaccacccccaatcg

SEQ ID NO: 59 - DNA, 25 bp, Artificial Sequence
  Primer
acacaccagaagaccaagatttacc

SEQ ID NO: 60 - DNA, 20 bp, Artificial Sequence
  Primer
tgctggagtgcgtttttcct

SEQ ID NO: 61 - DNA, 20 bp, Artificial Sequence
  Oligonucleotide for detection of amplified replication domain
cgccagtccttgctgggacc

SEQ ID NO: 62 - DNA, 21 bp, Artificial Sequence
  Oligonucleotide for detection of amplified replication domain
ccctgcccagaggacatcgcc

SEQ ID NO: 63 - DNA, 19 bp, Artificial Sequence
  Primer
caggggcataaggacaaag

SEQ ID NO: 64 - DNA, 20 bp, Artificial Sequence
  Primer
ggcttcctcagacccctctg

SEQ ID NO: 65 - DNA, 21 bp, Artificial Sequence
  Primer
cgagggtgatgaagaagagga

SEQ ID NO: 66 - DNA, 19 bp, Artificial Sequence
  Primer
cccctgagttgctgtgata

SEQ ID NO: 67 - DNA, 21 bp, Artificial Sequence
  Oligonucleotide for detection of amplified immobilization domain
tggtcctcatcttggccagca

SEQ ID NO: 68 - DNA, 20 bp, Artificial Sequence
  Oligonucleotide for detection of amplified immobilization domain
cacgggcaccaggaagtcca

SEQ ID NO: 69 - DNA, 20 bp, Artificial Sequence
  Oligonucleotide for detection of amplified immobilization domain
cactgcaagtccctgggcca

SEQ ID NO: 70 - protein, 33 aa, Artificial Sequence
  Peptide used to detect Ciz1 autoantibodies
EGDEEEEEDDEDEEEIEVRSRDISREEWKGSET

SEQ ID NO: 71 - protein, 41 aa, Artificial Sequence
  Peptide control sequence
EGDEEEEEDDEDEEEIEVEEELCKQVRSRDISREEWKGSET

SEQ ID NO: 72 - protein, 9 aa, Artificial Sequence
  Peptide blocking agent sequence
VEEELCKQV

SEQ ID NO: 73 - protein, 7 aa, Artificial Sequence
  Peptide blocking agent sequence
EEELCKQ

SEQ ID NO: 74 - DNA, 25 bp, Artificial Sequence
  Primer
tccgagcccttccactcctctctgg

SEQ ID NO: 75 - DNA, 22 bp, Artificial Sequence
  Primer
tcaggttttgaggcgggttgag

SEQ ID NO: 76 - DNA, 19 bp, Artificial Sequence
  Primer
ggttttgaggcgggttgag

SEQ ID NO: 77 - DNA, 19 bp, Artificial Sequence
  Primer
caaccgcgagaagatgacc

SEQ ID NO: 78 - DNA, 19 bp, Artificial Sequence
  Primer
tccagggcgacgtagcaca

SEQ ID NO: 79 - DNA, 19 bp, Artificial Sequence
  Primer
gaatctccagggcaccaac

SEQ ID NO: 80 - DNA, 19 bp, Artificial Sequence
  Primer
cgattgggggtggtagagg

SEQ ID NO: 81 - DNA, 20 bp, Artificial Sequence
  Primer
tgttgcatgagaaaacgcca

SEQ ID NO: 82 - DNA, 20 bp, Artificial Sequence
  Primer
gtcgcctgttcaccaaggat

SEQ ID NO: 83 - DNA, 20 bp, Artificial Sequence
  Oligonucleotide probe
ccctgtacgcctctggccgt

SEQ ID NO: 84 - DNA, 26 bp, Artificial Sequence
  Oligonucleotide probe
aagtgacagagtcaccaaatgctgca

SEQ ID NO: 85 - protein, 8 aa, Artificial Sequence
  Peptide used to detect Ciz1 autoantibodies
EEIEVRSR

(19) United States
(12) Patent Application Publication
(10) Pub. No.: US 2013/0210663 A1
(43) Pub. Date: Aug. 15, 2013

(54) METHODS AND COMPOUNDS FOR THE DIAGNOSIS AND TREATMENT OF CANCER

(71) Applicant: Dawn Alison Coverley, York (GB)

(72) Inventor: Dawn Alison Coverley, York (GB)

(73) Assignee: Cizzle Biotechnology Limited, York (GB)

(21) Appl. No.: 13/813,782

(22) PCT Filed: Aug. 4, 2011
(86) PCT No.: PCT/GB2011/001173

§ 371 Date: Apr. 11, 2013

Related U.S. Application Data
(60) Provisional application No. 61/370,479, filed on Aug. 4, 2010.
(60) Provisional application No. 61/372,981, filed on Aug. 12, 2010.
(60) Provisional application No. 61/442,823, filed on Feb. 15, 2011.

(51) Int. Cl. G01N 33/68; A61B 6/03; A61B 5/055; A61B 5/00

(52) U.S. Cl. CPC G01N 33/6893; A61B 5/004; A61B 6/032; A61B 5/055; A61B 6/037
USPC 506/9; 436/501; 530/387.9; 435/7.1; 435/7.92; 600/407; 600/436; 600/427; 600/411

(57) ABSTRACT

The present invention provides for methods for use in the diagnosis and prognosis of cancer. The invention further provides to binding agents and kits for us e.g., in such methods. The present invention further relates to compositions, methods of making said compositions and methods of using the same, including use in the treatment and diagnosis of cancer, including lung, lymphoma, liver, thyroid and bladder cancer. Compositions of the present invention useful in the treatment of cancer include anti-sense and small inhibitory RNAs (siRNA).

### RELATED APPLICATIONS

This application claims priority benefit of U.S. Provisional Application 61/370,479, filed 4 Aug. 2010, U.S. Provisional Application 61/372,981, filed 12 Aug. 2010 and U.S. Provisional Application 61/442,823, filed 15 Feb. 2011. This application is also is a continuation-in-part of PCT/GB2010/000204, filed 5 Feb. 2010, which in turn claims priority benefit of GB Application 0901837.5, filed 5 Feb. 2009.

### INCORPORATION BY REFERENCE

U.S. Provisional Applications 61/370,479, 61/372,981 and 61/442,823, PCT Application PCT/GB2010/000204 and GB Application 0901837.5 are herein incorporated by reference in their entireties.

### BACKGROUND

Cip1-interacting zinc finger protein 1 (Ciz1) (NCBI Reference Sequence: NM_{—}001131016.1) is required for cell proliferation. Ciz1 localises to nuclear matrix bound foci that form sites of DNA replication during early S phase and promotes the initiation of DNA replication in association with cell cycle regulators including cyclin A/CDK2, cyclin E/CDK2 and p21 cip1. In the context of transcription, CIZ1 is an oestrogen responsive gene that is itself a positive cofactor of the oestrogen receptor (ER), capable of enhancing the recruitment of ER to target chromatin. Ciz1 is alternatively spliced to produce conserved isoforms in mouse and man. Normal Ciz1 protein comprises at least two defined functional domains, a ‘replication’ domain and an ‘immobilisation’ domain.

The present invention relates, in part, to the discovery of alternative splicing of Ciz1 exon 14 in cancers, including small cell lung cancer (SCLC), non-small cell lung cancer (NSCLC), lymphomas, thyroid, kidney and liver cancer. The present invention further relates to the discovery of excess expression of either the replication or immobilisation domain in cancers including NSCLC, breast, colon, kidney, liver, bladder and thyroid cancers, and the correlation of domain expression with the stage of the cancer. The present invention addresses the continued need to develop diagnostic tests and treatments that improve the survival rates of patients suffering from cancers such as lung cancer through novel biomarkers and targets based on these molecular abnormalities in Ciz1 gene expression.

### SUMMARY

In one aspect, the present invention relates to a method of diagnosing cancer in a subject, said method comprising the steps of:
i) providing an isolated biological sample to be tested;
ii) detecting whether a Ciz1 b-variant polypeptide is present in said sample,

wherein the presence of said Ciz1 b-variant polypeptide indicates said subject has cancer.

In one embodiment, the cancer is selected from lung, lymphoma, kidney, breast, liver, bladder and thyroid cancer.

In one aspect, the present invention relates to a method for the early detection of lung cancer in a subject, said method comprising the steps of:
i) providing an isolated biological sample to be tested;
ii) detecting whether a Ciz1 b-variant polypeptide is present in said sample;

wherein the presence of said Ciz1 b-variant polypeptide in said sample indicates the subject has cancer.

In one aspect, the present invention relates to a method for the detection of lung cancer recurrence in a subject previously treated for lung cancer, said method comprising the steps of:
i) providing an isolated biological sample to be tested from said subject;
ii) detecting whether a Ciz1 b-variant polypeptide is present in said sample;

wherein the presence of said Ciz1 b-variant polypeptide in said sample indicates recurrence of lung cancer in said subject.

In one aspect, the present invention relates to a method of diagnosing cancer in a subject with a lung nodule, said method comprising the steps:
iii) providing an isolated biological sample to be tested;
iv) detecting whether a Ciz1 b-variant polypeptide is present in said sample;

wherein the presence of said Ciz1 b-variant polypeptide in said sample indicates the subject has cancer.

In one aspect, the present invention relates to a method of differentially diagnosing lung cancer from pneumonia in a subject suspected of having either pneumonia or lung cancer:
i) providing an isolated biological sample to be tested from said subject;
ii) detecting whether a Ciz1 b-variant polypeptide is present in said sample;

wherein the presence of said Ciz1 b-variant polypeptide in said sample indicates the subject has cancer.

In one embodiment of the methods of the invention, the cancer is non-small cell lung cancer (NSCLC). In another embodiment, the lung cancer is small cell lung cancer (SCLC). In another embodiment, the lung cancer is stage 0 NSCLC. In another ne embodiment, the lung cancer is stage IA NSCLC. In another ne embodiment, the lung cancer is stage IB NSCLC. In another embodiment, the lung cancer is limited stage SCLC.

In one embodiment of the methods, the lung nodule is less than about 20 mm in diameter. In another embodiment, the lung nodule is less than about 15 mm. In another embodiment, the lung nodule is less than or about 10 mm. In another embodiment, the lung nodule is less than about 7.5 mm. In another embodiment, the lung nodule is between about 5 mm to about 10 mm.

In one embodiment, the methods comprise the step of imaging the subject's lungs. In another embodiment, the imaging further comprises the step of performing a chest X-ray, computerized tomography (CT) scan, magnetic resonance imaging (MRI) scan or positron emission tomography (PET) scan, and wherein said imaging alone is insufficient for said diagnosing of cancer. In another embodiment, the imaging comprises the step of performing a chest X-ray. In another embodiment, the imaging comprises the step of performing a computerized tomography (CT) scan. In another embodiment, the CT scan is a low dose helical computerized tomography CT scan. In another embodiment, the imaging comprises the step of performing a MRI scan In another embodiment, the imaging comprises the step of performing a PET scan.

In one aspect, the present invention relates to a method of indicating cancer cell death in a subject treated for lung cancer, wherein said method comprises the steps of:
i) providing an isolated biological sample to be tested from said subject before and after said treatment;
ii) measuring an amount of said Ciz1 b-variant polypeptide present in said biological sample before and after said treatment;

wherein an increase in the amount of said Ciz1 b-variant polypeptide after treatment indicates tumor cell death.

In one embodiment of the methods, the Ciz1 b-variant polypeptide comprises the amino acid sequence DEEEIEVRSRDIS (SEQ ID NO: 8). In another embodiment, the Ciz1 b-variant polypeptide comprises the amino acid sequence of SEQ ID NO: 22.

In one embodiment of the methods, the biological sample is tissue, blood, plasma, sputum, bronchoalveolar lavage or urine. In another embodiment, the biological sample is tissue. In another embodiment, the tissue is lung tissue. In another embodiment, the biological sample is blood. In another embodiment, the biological sample is an isolated CTC. In another embodiment, the biological sample is plasma. In another embodiment, the biological sample is sputum. In another embodiment, the biological sample is bronchoalveolar lavage. In another embodiment, the biological sample is urine. In one embodiment of the methods of the invention, the Ciz1 b-variant polypeptide is extracellular.

In one embodiment of the methods, less than 100 μL of said biological sample is tested for the presence of said Ciz1 b-variant polypeptide. In another embodiment, less than 50 μL of said biological sample is tested for the presence of said Ciz1 b-variant polypeptide. In another embodiment, less than 25 μL of said biological sample is tested for the presence of said Ciz1 b-variant polypeptide. In another embodiment, less than 10 μL of said biological sample is tested for the presence of said Ciz1 b-variant polypeptide.

In another embodiment, less than 5 μL of said biological sample is tested for the presence of said Ciz1 b-variant polypeptide. In another embodiment, less than 1 μL of said biological sample is tested for the presence of said Ciz1 b-variant polypeptide. In another embodiment, between 0.5-5 μL of said biological sample is tested for the presence of said Ciz1 b-variant polypeptide. In another embodiment, between 0.25-5 μL of said biological sample is tested for the presence of said Ciz1 b-variant polypeptide. In another embodiment, between 0.25-2 μL of said biological sample is tested for the presence of said Ciz1 b-variant polypeptide. In another embodiment, between 0.5-1.5 μL of said biological sample is tested for the presence of said Ciz1 b-variant polypeptide. In another embodiment, about 1 μL of biological sample is tested for the presence of said Ciz1 b-variant polypeptide.

In one embodiment, the methods further comprise the step of contacting said biological sample with a Ciz1 b-variant polypeptide binding agent. In another embodiment, the Ciz1 b-variant polypeptide binding agent is an antibody or antigen binding fragment thereof. In another embodiment, the antibody is polyclonal. In another embodiment, the antibody is monoclonal. In another embodiment, the antigen binding fragment is selected from a Fab, Fab′, F(ab′)₂, scFv or sdAb. In another embodiment, the Ciz1 b-variant polypeptide binding agent is a nucleic acid aptamer. In another embodiment, the Ciz1 b-variant polypeptide binding agent is a peptide aptamer. In another embodiment, the Ciz1 b-variant polypeptide binding agent is a peptidomimetic.

In one embodiment of the methods, the Ciz1 b-variant polypeptide binding agent specifically binds a Ciz1 b-variant polypeptide comprising the amino acid sequence SEQ ID NO: 22. In another embodiment, the Ciz1 b-variant polypeptide binding agent specifically binds a Ciz1 b-variant polypeptide comprising the amino acid sequence of SEQ ID NO: 8. In another embodiment, the Ciz1 b-variant polypeptide binding agent specifically binds an epitope spanning exons 14b and 15. In another embodiment, the binding agent specifically binds a Ciz1 b-variant polypeptide comprising the amino acid sequence of SEQ ID NO: 8 with at least 100 fold greater affinity than a Ciz1 polypeptide comprising the amino acid sequence of SEQ ID NO: 23. In another embodiment, the binding agent specifically binds said Ciz1 b-variant polypeptide with at least 1,000 fold greater affinity than said Ciz1 polypeptide. In another embodiment, the binding agent specifically binds said Ciz1 b-variant polypeptide with at least 10,000 fold greater affinity than said Ciz1 polypeptide. In another embodiment, the binding agent does not specifically bind the amino acid sequence of SEQ ID NO: 23.

In one embodiment, the methods comprise the step contacting said biological sample with a second Ciz1 b-variant polypeptide binding agent, wherein said second Ciz1 b-variant polypeptide binding agent recognizes an epitope other than an epitope spanning exons 14b and 15. In another embodiment, second the Ciz1 b-variant polypeptide binding agent is an antibody or antigen binding fragment thereof. In another embodiment, the antibody is polyclonal. In another embodiment, the antibody is monoclonal. In another embodiment, the antigen binding fragment is selected from a Fab, Fab′, F(ab′)₂, scFv or sdAb. In another embodiment, the second Ciz1 b-variant polypeptide binding agent is a nucleic acid aptamer. In another embodiment, the second Ciz1 b-variant polypeptide binding agent is a peptide aptamer. In another embodiment, the second Ciz1 b-variant polypeptide binding agent is a peptidomimetic.

In one embodiment, the methods further comprise the step of immobilizing said Ciz1 b-variant polypeptide on a solid support. In another embodiment, the solid support is a bead. In another embodiment, the solid support is a microtiter plate. In another embodiment, the further comprises the step of immobilizing said second Ciz1 b-variant polypeptide binding agent on a solid support. In another embodiment, the second Ciz1 b-variant polypeptide binding agent immobilizes said Ciz1 b-variant polypeptide on said solid support when bound thereto. In another embodiment, the method is a sandwich assay. In another embodiment, the method is a sandwich immunoassay. In another embodiment, the method is an ELISA.

In one aspect, the present invention relates to an isolated Ciz1 b-variant polypeptide binding agent that specifically binds a Ciz1 b-variant polypeptide.

In one embodiment, the Ciz1 b-variant polypeptide binding agent specifically binds a Ciz1 b-variant polypeptide comprising the amino acid sequence SEQ ID NO: 22. In another embodiment, the Ciz1 b-variant polypeptide binding agent specifically binds a Ciz1 b-variant polypeptide comprising the amino acid sequence of SEQ ID NO: 8. In another embodiment, the Ciz1 b-variant polypeptide binding agent specifically binds an epitope spanning exons 14b and 15. In another embodiment, the binding agent specifically binds a Ciz1 b-variant polypeptide comprising the amino acid sequence of SEQ ID NO: 8 with at least 100 fold greater affinity than a Ciz1 polypeptide comprising the amino acid sequence of SEQ ID NO: 23. In another embodiment, the binding agent specifically binds said Ciz1 b-variant polypeptide with at least 1,000 fold greater affinity than said Ciz1 polypeptide. In another embodiment, the binding agent specifically binds said Ciz1 b-variant polypeptide with at least 10,000 fold greater affinity than said Ciz1 polypeptide. In another embodiment, the binding agent does not specifically bind the amino acid sequence of SEQ ID NO: 23. In another embodiment, the binding agent is an isolated antibody or antigen binding fragment thereof. In another embodiment, the antibody is polyclonal. In another embodiment, the antibody is monoclonal. In another embodiment, the antigen binding fragment is selected from a Fab, Fab′, F(ab′)₂, scFv or sdAb. In another embodiment, the binding agent is a nucleic acid aptamer. In another embodiment, the binding agent is a peptide aptamer. In another embodiment, the binding agent is a peptidomimetic.

In one aspect, the invention relates to an isolated cell expressing the Ciz1 b-variant polypeptide binding agent of the invention.

In one aspect, the present invention relates to an isolated human autoantibody that specifically binds a Ciz1 b-variant polypeptide.

In one aspect, the present invention relates to a method of diagnosing cancer in a subject comprising the steps of:
i) providing an isolated biological sample to be tested;
ii) determining whether a Ciz1 b-variant transcript is present in said biological sample, wherein the presence of said Ciz1 b-variant transcript indicates the presence of cancer cells in said biological sample.

In one aspect, the present invention relates to a method of diagnosing cancer in a subject by comparing expression a Ciz 1 replication domain to a Ciz 1 immobilisation domain, said method comprising the steps of:
i) providing an isolated biological sample to be tested;
ii) detecting mRNA comprising a nucleotide sequence encoding Ciz 1 replication domain;
iii) detecting mRNA comprising a nucleotide sequence encoding Ciz 1 immobilisation domain;
iv) comparing relative expression levels of said mRNA comprising a nucleotide sequence encoding said Ciz 1 replication domain to said mRNA comprising a nucleotide sequence encoding said Ciz 1 immobilisation domain; wherein a difference in relative expression of at least 2 fold indicates the presence of cancer cells.

In one aspect, the present invention relates to a method of diagnosing cancer in a subject by comparing the expression of a polypeptide comprising a Ciz 1 replication domain to a polypeptide comprising a Ciz 1 immobilisation domain, said method comprising the steps of:
i) providing an isolated biological sample to be tested;
ii) detecting said Ciz 1 replication domain and said Ciz 1 immobilisation domain;
iii) comparing relative levels of said Ciz 1 replication domain to said Ciz 1 immobilisation domain present in said sample; wherein a difference of greater than 2 fold in the relative level of Ciz 1 replication domain to said Ciz 1 immobilisation domain indicates the presence of cancer.

In one aspect, the present invention relates to a method for indicating prognosis of a cancer patient by comparing expression a Ciz 1 replication domain to a Ciz 1 immobilisation domain, said method comprising the steps of:
i) providing an isolated biological solid tissue sample to be tested, where said tissue is adjacent to a solid tumor;
ii) detecting mRNA comprising a nucleotide sequence encoding Ciz 1 replication domain;
iii) detecting mRNA comprising a nucleotide sequence encoding Ciz 1 immobilisation domain;
iv) comparing relative expression levels of said mRNA comprising a nucleotide sequence encoding said Ciz 1 replication domain to said mRNA comprising a nucleotide sequence encoding said Ciz 1 immobilisation domain; wherein a difference in relative expression of at least 2 fold indicates a poorer prognosis.

In one aspect, the present invention relates to a method for indicating prognosis of a cancer patient by comparing the expression of a polypeptide comprising a Ciz 1 replication domain to a polypeptide comprising a Ciz 1 immobilisation domain, said method comprising the steps of:
i) providing an isolated biological solid tissue sample to be tested, where said tissue is adjacent to a solid tumor;
ii) detecting said Ciz 1 replication domain and said Ciz 1 immobilisation domain in said tissue sample;
iii) comparing relative levels of said Ciz 1 replication domain to said Ciz 1 immobilisation domain present in said sample; wherein a difference of greater than 2 fold in the relative level of Ciz 1 replication domain to said Ciz 1 immobilisation domain indicates a poorer prognosis.

In one aspect, the present invention relates to a method for diagnosis or prognosis of cancer in a subject comprising the steps of: (a) quantitatively detecting a Ciz1 protein in a biological sample derived from a subject; and (b) comparing the level of said Ciz1 protein detected in the subject's sample to the level of protein detected in a control sample, wherein an increase in the level of Ciz1 protein detected in the subject's sample as compared to a control sample is an indicator of a subject with cancer.

In one aspect, the present invention relates to a method for detecting an anti-Ciz1 antibody in a biological sample comprising the steps of: (a) contacting an anti-Ciz1 antibody containing sample with a sample containing a Ciz1 protein antigen under conditions such that an immunospecific antigen-antibody binding reaction can occur; and (b) detecting immunospecific binding of the anti-Ciz1 antibody to the Ciz1 protein in the sample.

In one embodiment, the methods comprise the step of detecting the anti-Ciz1 antibody in the sample comprises using a signal-generating component bound to an antibody that is specific for anti-Ciz1 antibody in the sample. In another embodiment, the presence of anti-Ciz1 antibody in the sample is measured by an immunoassay comprising the steps of: (a) immobilizing one or more Ciz1 protein onto a solid substrate; (b) contacting the solid substrate with the sample; and (c) detecting the presence of anti-Ciz1 antibody specific for the Ciz1 protein in the sample

In one aspect, the present invention relates to a kit for diagnosis and prognosis of cancer in a subject comprising a component for detecting the presence of a Ciz1 polypeptide in a biological sample. In one embodiment of the kit, the component for detecting the presence of a Ciz1 polypeptide is a Ciz1 binding agent. In another embodiment, the Ciz1 polypeptide is a Ciz1 b-variant polypeptide. In another embodiment, the component for detecting the Ciz1 polypeptide is an anti-Ciz1 antibody. In another embodiment, the anti-Ciz1 antibody is labeled. In another embodiment, the label is radioactive, fluorescent, colorimeter or enzyme label. In another embodiment, the kit comprises a labeled second antibody that immunospecifically binds to the anti-Ciz1 antibody.

In one aspect, the present invention relates to a kit for detecting the presence of an anti-Ciz1 autoantibody in a biological sample comprising a component for detecting the presence of said anti-Ciz1 antibody in said biological sample. In one embodiment of the kit, the component is a Ciz1 antigen. In another embodiment, the Ciz1 antigen is labeled. In another embodiment, the Ciz1 antigen is linked to a solid phase.

The present invention further relates to compositions, methods of making said compositions and methods of using the same, including use in the treatment and diagnosis of cancer.

In one aspect the present invention is directed to an antisense oligonucleotide or a siRNA or shRNA that targets a mRNA of Ciz1 comprising a variant of exon 14 referred to herein as exon 14b (SEQ ID NO: 3). Ciz1 exon 14b lacks 24 nucleotides at the 3′ end as compared to full length exon 14, referred to as exon 14a (SEQ ID NO: 1). Ciz1 transcripts expressing exon 14b rather than exon 14a (a-variant) are referred to as Ciz1 b-variant or simply b-variant.

Various aspects of this invention provide compounds suitable for reducing the expression of a b-variant transcript in cells.

In one aspect, the invention provides an antisense oligonucleotide that targets a Ciz1 b-variant transcript through a nucleotide sequence of Ciz1 that spans the junction of exons 14b and 15 (nucleotides 25-26 of SEQ ID NO: 7).

In another aspect, the invention provides an siRNA or shRNA that targets a Ciz1 b-variant transcript through a nucleotide sequence of Ciz1 that spans the junction of exons 14b and 15 (nucleotides 25-26 of SEQ ID NO: 7).

In another aspect, the invention provides a composition comprising an antisense oligonucleotide according to the present invention.

In another aspect, the invention provides a composition comprising a siRNA or shRNA according to the present invention.

In another aspect, the invention provides a pharmaceutical composition comprising an antisense oligonucleotide according to the invention and a pharmaceutically acceptable excipient.

In another aspect, the invention provides a pharmaceutical composition comprising a siRNA or shRNA according to the invention and a pharmaceutically acceptable excipient.

In another aspect, the invention provides a method of reducing expression of a b-variant transcript in a cell, comprising the step of contacting a cell expressing a b-variant transcript with a b-variant reducing amount of an antisense oligonucleotide, siRNA or shRNA according to the invention. In another aspect, the invention provides a method of reducing expression of a b-variant transcript in a non-human mammal, comprising the step of administering to the mammal a b-variant reducing amount of a composition comprising an antisense oligonucleotide, siRNA or shRNA according to the invention.

In another aspect, the invention provides a method of reducing expression of a b-variant transcript in a human, comprising the step of administering to the human a b-variant reducing amount of a composition comprising an antisense oligonucleotide, siRNA or shRNA according to the invention.

In one embodiment the antisense oligonucleotide, siRNA or shRNA of the present invention reduces expression of a Ciz1 b-variant transcript in a human or human cell, but not a Ciz1 transcript comprising exon 14a. In another aspect, the invention provides for a method of detecting a b-variant transcript, said method comprising the steps of contacting a b-variant transcript with a nucleic acid complementary to all or a portion of said b-variant transcript under conditions suitable for hybridization between said b-variant transcript and said nucleic acid to occur, and detecting said nucleic acid bound to said b-variant transcript. In one embodiment, the nucleic acid is an antisense oligonucleotide of the present invention or comprises the nucleic acid sequence of an antisense oligonucleotide of the present invention. In one embodiment the nucleic acid complementary to said b-variant transcript hybridizes to all or a portion of said b-variant transcript that includes a nucleotide sequence of Ciz1 that spans the junction of exons 14b and 15 (nucleotides 25-26 of SEQ ID NO: 7). In one embodiment the nucleic acid complementary to said b-variant transcript hybridizes to all or a portion of the nucleotide sequence of SEQ ID NO: 7, including nucleotides 25-26 of SEQ ID NO: 7. In one embodiment the antisense oligonucleotide hybridizes to a b-variant but not an a-variant transcript.

In another aspect the invention provides for methods of making the compounds of the present invention.

### Brief Description of the Sequences

SEQ ID NO: 1 is the nucleotide sequence of full length Ciz1 exon 14, referred to as exon 14a.

SEQ ID NO: 2 is the polypeptide sequence of full length Ciz1 exon 14, referred to as exon 14a.

SEQ ID NO: 3 is the nucleotide sequence of a variant of Ciz1 exon 14, lacking 24 nucleotides at the 3′-end of exon 14, referred to herein as exon 14b.

SEQ ID NO: 4 is the amino acid sequence of a variant Ciz1 exon 14, lacking 8 amino acid residues at the COOH-end of exon 14, referred to as exon 14b.

SEQ ID NO: 5 is the nucleotide sequence of Ciz1 exon 15.

SEQ ID NO: 6 is the amino acid sequence of Ciz1 exon 15.

SEQ ID NO: 7 is the nucleotide sequence of a portion of Ciz1 b-variant transcript spanning the splice junction of exons 14b and 15.

SEQ ID NO: 8 is the amino acid sequence of a portion of Ciz1 b-variant polypeptide spanning the splice junction of exons 14b and 15.

SEQ ID NO: 9 is the amino acid sequence of the replication domain (met in exon 3 to end of exon 9).

SEQ ID NO: 10 is the amino acid sequence of a portion of the replication domain (exons 5-9).

SEQ ID NO: 11 is the amino acid sequence of a further restricted portion of the replication domain (exons 5-9, excluding internal part of exon 8).

SEQ ID NO: 12 is the nucleotide sequence of the replication domain (met in exon 3 to end of exon 9).

SEQ ID NO: 13 is the nucleotide sequence of a portion of the replication domain (exons 5-9).

SEQ ID NO: 14 is the nucleotide sequence of a further restricted portion of the replication domain (exons 5-9, excluding internal part of exon 8).

SEQ ID NO: 15 is the amino acid sequence of the immobilisation domain SEQ ID NO: 16 is the amino acid sequence of a portion of the immobilisation domain.

SEQ ID NO: 17 is the amino acid sequence of a further restricted portion of the immobilisation domain.

SEQ ID NO: 18 is the nucleotide sequence of the immobilisation domain SEQ ID NO: 19 is the nucleotide sequence of a portion of the immobilisation domain SEQ ID NO: 20 is the nucleotide sequence of a further restricted portion of the immobilisation domain SEQ ID NO: 21 is the amino acid sequence of exons 14a and 15.

SEQ ID NO: 22 is the amino acid sequence of exons 14b and 15.

SEQ ID NO: 23 is the amino acid sequence of a portion of a Ciz1 a-variant polypeptide spanning the splice junction of exons 14a and 15).

### DETAILED DESCRIPTION

The present invention relates to compounds and compositions as well as methods of making said compounds and compositions and methods of using the same. The compounds and compositions of the present invention are, e.g., useful in the treatment and diagnosis on cancers including cancers of the lung, breast, colon, kidney, liver and lymphomas.

In one aspect the present invention is directed to an antisense oligonucleotide, siRNA or shRNA that targets only b-variant transcripts of Ciz1.

In another aspect the present invention is directed to a composition comprising an antisense oligonucleotide, siRNA or shRNA that targets only b-variant transcripts of Ciz1.

In another aspect the present invention is directed to a pharmaceutical composition comprising an antisense oligonucleotide, siRNA or shRNA according to the invention and a pharmaceutically acceptable excipient.

In another aspect the present invention is directed to methods of using the siRNA or shRNA to reduce the expression level of a Ciz1 b-variant transcript. As used herein, the terms “silence” or “knock-down” when referring to gene expression means a reduction in gene expression. The term “transcript” refers to an RNA product of transcription. In one embodiment, a transcript is an mRNA.

The present invention further relates to processes for making an antisense oligonucleotide, siRNA or shRNA of the present invention by chemical synthesis.

The antisense oligonucleotides of the present invention are suitable to detect the expression of a Ciz1 b-variant transcript. In one aspect the antisense oligonucleotides are suitable to reduce the level of a Ciz1 b-variant transcript in a mammalian cell. The antisense oligonucleotides according to the present invention are further suitable to decrease the expression of a Ciz1 b-variant protein encoded by a Ciz1 b-variant mRNA by decreasing gene expression at the level of mRNA.

The siRNA or shRNA of the present invention are suitable to reduce the level of a Ciz1 b-variant transcript. The siRNA or shRNA according to the present invention are further suitable to decrease the expression of protein encoded by a Ciz1 b-variant mRNA by decreasing gene expression at the level of mRNA.

Antisense Design:

An antisense oligonucleotide suitable to reduce the level of a Ciz1 b-variant transcript is a single stranded oligonucleotide 12 to 50 nucleotides in length comprising at least 8 contiguous nucleotides complementary to SEQ ID NO: 7, including nucleotides at positions 25-26 of SEQ ID NO:7.

In one embodiment, the complementarity between an antisense oligonucleotide and SEQ ID NO:7 is such that the antisense oligonucleotide can hybridize to a sequence of SEQ ID NO:7, including nucleotides at positions 25-26 of SEQ ID NO: 7, under stringent hybridization conditions, wherein ‘stringent hybridization’ is defined herein as the following hybridization conditions: 400 mM NaCl, 40 mM PIPES pH 6.4, 1 mM EDTA, 70° C.

The nucleotides of the antisense oligonucleotide may be deoxyribonucleotides, ribonucleotides, modified ribonucleotides or a combination thereof. When the antisense oligonucleotide is used to degrade mRNA through RNaseH, normally at least some of the nucleotides are deoxyribonucleotides.

siRNA Design:

An siRNA of the present invention comprises two strands of nucleic acid, a first, antisense strand and a second, sense strand. The nucleic acid normally consists of ribonucleotides or modified ribonucleotides however; the nucleic acid may comprise deoxyribonucleotides (DNA). The siRNA further comprises a double-stranded nucleic acid portion or duplex region formed by all or a portion of the antisense strand and all or a portion of the sense strand. The portion of the antisense strand forming the duplex region with the sense strand is the antisense strand duplex region or simply, the antisense duplex region, and the portion of the sense strand forming the duplex region with the antisense strand is the sense strand duplex region or simply, the sense duplex region. The duplex region is defined as beginning with the first base pair formed between the antisense strand and the sense strand and ending with the last base pair formed between the antisense strand and the sense strand, inclusive. The portion of the siRNA on either side of the duplex region is the flanking regions. The portion of the antisense strand on either side of the antisense duplex region is the antisense flanking regions. The portion of the antisense strand 5′ to the antisense duplex region is the antisense 5′ flanking region. The portion of the antisense strand 3′ to the antisense duplex region is the antisense 3′ flanking region. The portion of the sense strand on either side of the sense duplex region is the sense flanking regions. The portion of the sense strand 5′ to the sense duplex region is the sense 5′ flanking region. The portion of the sense strand 3′ to the sense duplex region is the sense 3′ flanking region.

Complementarity.

In one aspect, the antisense duplex region and the sense duplex region may be fully complementary and are at least partially complementary to each other. Such complementarity is based on Watson-Crick base pairing (i.e., A:U and G:C base pairing). Depending on the length of a siRNA a perfect match in terms of base complementarity between the antisense and sense duplex regions is not necessarily required however, the antisense and sense strands must be able to hybridize under physiological conditions. In one embodiment, the complementarity between the antisense strand and sense strand is perfect (no nucleotide mismatches or additional/deleted nucleotides in either strand). In one embodiment, the complementarity between the antisense duplex region and sense duplex region is perfect (no nucleotide mismatches or additional/deleted nucleotides in the duplex region of either strand). In another embodiment, the complementarity between the antisense duplex region and the sense duplex region is not perfect.

RNAi using siRNA or shRNA or other related designs of the present invention involves the formation of a duplex region between all or a portion of the antisense strand and a portion of the nucleotide sequence of SEQ ID NO:7, including nucleotides at position 25-26 (the ‘target nucleic acid’ or ‘target sequence’). More specifically, the ‘target sequence’ is the portion of SEQ ID NO:7, including nucleotides at position 25-26, that forms a duplex region with the antisense strand, defined as beginning with the first base pair formed between the antisense strand and SEQ ID NO:7 and ending with the last base pair formed between the antisense strand and the SEQ ID NO:7.

The duplex region formed between the antisense strand and the sense strand may, but need not be the same as the duplex region formed between the antisense strand and the target sequence. That is, the sense strand may have a sequence different from the target nucleic acid however; the antisense strand must be able to form a duplex structure under physiological conditions with both the sense strand and the target nucleic acid.

In one embodiment, the complementarity between the antisense strand and the target nucleic acid is perfect (no nucleotide mismatches or additional/deleted nucleotides in either nucleic acid). In one embodiment, the complementarity between the antisense duplex region (the portion of the antisense strand forming a duplex region with the sense strand) and the target nucleic acid is perfect (no nucleotide mismatches or additional/deleted nucleotides in either nucleic acid). In another embodiment, the complementarity between the antisense duplex region and the target nucleic acid is not perfect.

In another embodiment, the siRNA of the invention comprises a duplex region wherein the antisense duplex region has 1, 2 or 3 nucleotides that are not base-paired to a nucleotide in the sense duplex region, and wherein said siRNA is suitable for reducing expression of a b-variant transcript. In another embodiment, the antisense strand has 1, 2 or 3 nucleotides that do not base-pair to the sense strand, and wherein a siRNA comprising said antisense strand is suitable for reducing expression of a b-variant transcript. Lack of base-pairing is due to either lack of complementarity between bases (i.e., no Watson-Crick base pairing) or because there is no corresponding nucleotide such that a bulge or overhang is created.

In another embodiment, the antisense duplex region and sense duplex region hybridize under stringent hybridization conditions, wherein ‘stringent hybridization conditions’ is defined as: 400 mM NaCl, 40 mM PIPES pH 6.4, 1 mM EDTA, 70° C. In another embodiment, the antisense duplex region and the target nucleic acid hybridize under stringent hybridization conditions. In another embodiment, the antisense duplex region and both the sense duplex region and the target nucleic acid hybridize under stringent hybridization conditions.

Like the siRNA of the present invention, the antisense oligonucleotides of the present invention may be fully complementary and are at least partially complementary to the target nucleic acid. A perfect match in terms of base complementarity between the antisense oligonucleotide and target nucleic acid is not necessarily required however, the antisense oligonucleotide and target nucleic acid must be able to hybridize under physiological conditions. In one embodiment, the complementarity between the antisense oligonucleotide and target nucleic acid is perfect (no nucleotide mismatches or additional/deleted nucleotides in either strand). In another embodiment, the complementarity between the antisense oligonucleotide and target nucleic acid is not perfect. In another embodiment, the antisense oligonucleotide and the target nucleic sequence hybridize under stringent hybridization conditions.

Length:

An aspect of the present invention relates to the length of the nucleic acid and particular regions that make up the antisense oligonucleotide or siRNA.

In certain embodiments the present invention relates to an antisense oligonucleotide 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49 or 50 nucleotides in length comprising at least 8 contiguous nucleotides complementary to SEQ ID NO:7 and includes nucleotides 25-26.

In certain embodiments the present invention relates to an isolated antisense oligonucleotide comprising 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49 or 50 contiguous nucleotides complementary to SEQ ID NO:7 and includes nucleotides 25-26.

In certain embodiments the present invention relates to an antisense oligonucleotide consisting of 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49 or 50 contiguous nucleotides complementary to SEQ ID NO:7 and includes nucleotides 25-26 of SEQ ID NO:7.

In one embodiment the present invention relates to a siRNA comprising an antisense strand and a sense strand;

wherein said antisense strand and said sense strand are each independently less than or equal to 30 nucleotides in length;

wherein said sense strand comprises a sense duplex region;

wherein said sense duplex region comprises a nucleotide sequence comprising at least 16 contiguous nucleotides of SEQ ID NO:7, wherein said contiguous nucleotides includes nucleotides 25-26 of SEQ ID NO: 7;

wherein said antisense strand comprises an antisense duplex region;

wherein said antisense duplex region has a nucleotide length equal to said sense duplex region; and

wherein said antisense duplex region comprises a nucleotide sequence complementary to said sense duplex region.

In one embodiment the present invention relates to a siRNA comprising an antisense strand and a sense strand;

wherein said antisense strand and said sense strand are each independently less than or equal to 30 nucleotides in length;

wherein said sense strand comprises a sense duplex region;

wherein said sense duplex region comprises a nucleotide sequence comprising at least 18 contiguous nucleotides of SEQ ID NO:7, wherein said contiguous nucleotides includes nucleotides 25-26 of SEQ ID NO: 7;

wherein said antisense strand comprises an antisense duplex region;

wherein said antisense duplex region has a nucleotide length equal to said sense duplex region; and

wherein said antisense duplex region comprises a nucleotide sequence complementary to said sense duplex region.

In one embodiment the present invention relates to a siRNA comprising an antisense strand and a sense strand;

wherein said antisense strand and said sense strand are each independently less than or equal to 25 nucleotides in length;

wherein said sense strand comprises a sense duplex region;

wherein said sense duplex region comprises a nucleotide sequence comprising at least 16 contiguous nucleotides of SEQ ID NO: 7, wherein said contiguous nucleotides includes nucleotides 25-26 of SEQ ID NO: 7;

wherein said antisense strand comprises an antisense duplex region;

wherein said antisense duplex region has a nucleotide length equal to said sense duplex region; and

wherein said antisense duplex region comprises a nucleotide sequence complementary to said sense duplex region.

In one embodiment the present invention relates to a siRNA comprising an antisense strand and a sense strand;

wherein said antisense strand and said sense strand are each independently less than or equal to 25 nucleotides in length;

wherein said sense strand comprises a sense duplex region;

wherein said sense duplex region comprises a nucleotide sequence comprising at least 18 contiguous nucleotides of SEQ ID NO:7, wherein said contiguous nucleotides includes nucleotides 25-26 of SEQ ID NO: 7;

wherein said antisense strand comprises an antisense duplex region;

wherein said antisense duplex region has a nucleotide length equal to said sense duplex region; and

wherein said antisense duplex region comprises a nucleotide sequence complementary to said sense duplex region.

In one embodiment the present invention relates to a siRNA comprising an antisense strand and a sense strand;

wherein said antisense strand and said sense strand are each independently 18-25 nucleotides in length;

wherein said sense strand comprises a sense duplex region;

wherein said sense duplex region comprises a nucleotide sequence comprising at least 16 contiguous nucleotides of SEQ ID NO:7, wherein said contiguous nucleotides includes nucleotides 25-26 of SEQ ID NO: 7;

wherein said antisense strand comprises an antisense duplex region;

wherein said antisense duplex region has a nucleotide length equal to said sense duplex region; and

wherein said antisense duplex region comprises a nucleotide sequence complementary to said sense duplex region.

In one embodiment the present invention relates to a siRNA comprising an antisense strand and a sense strand;

wherein said antisense strand and said sense strand are each independently 18-25 nucleotides in length;

wherein said sense strand comprises a sense duplex region;

wherein said sense duplex region comprises a nucleotide sequence comprising at least 18 contiguous nucleotides of SEQ ID NO:7, wherein said continuous nucleotides includes nucleotides 25-26 of SEQ ID NO: 7;

wherein said antisense strand comprises an antisense duplex region;

wherein said antisense duplex region has a nucleotide length equal to said sense duplex region; and

wherein said antisense duplex region comprises a nucleotide sequence complementary to said sense duplex region.

In one embodiment the present invention relates to a siRNA comprising an antisense strand and a sense strand;

wherein said antisense strand and said sense strand are each independently 19-23 nucleotides in length;

wherein said sense strand comprises a sense duplex region;

wherein said sense duplex region comprises a nucleotide sequence comprising at least 18 contiguous nucleotides of SEQ ID NO:7, wherein said continuous nucleotides includes nucleotides 25-26 of SEQ ID NO: 7;

wherein said antisense strand comprises an antisense duplex region;

wherein said antisense duplex region has a nucleotide length equal to said sense duplex region; and

wherein said antisense duplex region comprises a nucleotide sequence complementary to said sense duplex region.

In one embodiment the present invention relates to a siRNA comprising an antisense strand and a sense strand;

wherein said antisense strand and said sense strand are each 19-25 nucleotides in length;

wherein said sense strand comprises a sense duplex region;

wherein said sense duplex region comprises a nucleotide sequence comprising at least 19 contiguous nucleotides of SEQ ID NO:7, wherein said continuous nucleotides includes nucleotides 25-26 of SEQ ID NO: 7;

wherein said antisense strand comprises an antisense duplex region;

wherein said antisense duplex region has a nucleotide length equal to said sense duplex region; and

wherein said antisense duplex region comprises a nucleotide sequence complementary to said sense duplex region.

In one embodiment the present invention relates to a siRNA comprising an antisense strand and a sense strand;

wherein said antisense strand and said sense strand are each 19-23 nucleotides in length;

wherein said sense strand comprises a sense duplex region;

wherein said sense duplex region comprises a nucleotide sequence comprising at least 19 contiguous nucleotides of SEQ ID NO:7, wherein said continuous nucleotides includes nucleotides 25-26 of SEQ ID NO: 7

wherein said antisense strand comprises an antisense duplex region;

wherein said antisense duplex region has a nucleotide length equal to said sense duplex region; and

wherein said antisense duplex region comprises a nucleotide sequence complementary to said sense duplex region.

In one embodiment the antisense strand of an siRNA or shRNA of the present invention comprises a nucleotide sequence complementary to a nucleotide sequence selected from:

5′ AAGAAGAGAUCGAGGUGAGGU 3′;
5′ AAGAGAUCGAGGUGAGGUCCA 3′;
5′ AGAAGAGAUCGAGGUGAGGUC 3′;
5′ GAAGAGAUCGAGGUGAGGUCC 3′;
or
5′ AGAGAUCGAGGUGAGGUCCAG 3′.

Ends (overhangs and blunt ends):

Another aspect relates to the end design of the siRNA. The siRNA of the present invention may comprise an overhang or be blunt ended. An “overhang” as used herein has its normal and customary meaning in the art, i.e., a single stranded portion of a nucleic acid that extends beyond the terminal nucleotide of a complementary strand in a double strand nucleic acid. The term “blunt end” includes double stranded nucleic acid whereby both strands terminate at the same position, regardless of whether the terminal nucleotide(s) are base paired.

In one embodiment, the terminal nucleotides of a blunt end are base paired.

In another embodiment, the terminal nucleotides of a blunt end are not paired.

In one embodiment, the siRNA of the present invention has an overhang of 1, 2, 3, 4 or 5 nucleotides at one end and a blunt end at the other end.

In another embodiment, the siRNA has an overhang of 1, 2, 3, 4 or 5 nucleotides at both ends.

In one embodiment, the siRNA is blunt ended at both ends.

In another embodiment, the siRNA is blunt ended at the end defined by the 5′-end of the sense strand and the 3′-end of the antisense strand.

In another embodiment, the siRNA is blunt ended at the end defined by the 3′-end of the sense strand and the 5′-end of the antisense strand.

In another embodiment, the siRNA comprises a overhang of 1, 2, 3, 4 or 5 nucleotides at a 3′- or 5′-end on either or both the sense and antisense strands.

In one embodiment, the siRNA has a 3′-overhang of 1, 2, 3, 4 or 5 nucleotides on the antisense strand and is blunt ended at the other end.

In one embodiment, the siRNA has a 3′-overhang of 1, 2, 3, 4 or 5 nucleotides on the sense strand and is blunt ended at the other end.

In one embodiment, the siRNA has a 5′-overhang of 1, 2, 3, 4 or 5 nucleotides on the antisense strand and is blunt ended at the other end.

In one embodiment, the siRNA has a 5′-overhang of 1, 2, 3, 4 or 5 nucleotides on the sense strand and is blunt ended at the other end.

In one embodiment, the siRNA has a 3′-overhang of 1, 2, 3, 4 or 5 nucleotides on the antisense strand and a 3′-overhang of 1, 2, 3, 4 or 5 nucleotides on the sense strand.

In one embodiment, the siRNA has a 5′-overhang of 1, 2, 3, 4 or 5 nucleotides on the antisense strand and a 5′-overhang of 1, 2, 3, 4 or 5 nucleotides on the sense strand.

Modifications to Base Moiety:

Another aspect relates to modifications to a base moiety. One or more nucleotides of a nucleic acid of the present invention may comprise a modified base. A “modified base” means a nucleotide base other than an adenine, guanine, cytosine or uracil at the 1′ position.

In one embodiment, the antisense oligonucleotide, siRNA or shRNA of the present invention comprises at least one nucleotide comprising a modified base.

In another embodiment, the nucleic acid of the present invention comprises a modified nucleotide, wherein the modified nucleotide comprises a modified base, wherein the modified base is selected from 2-aminoadenosine, 2,6-diaminopurine, inosine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidine (e.g., 5-methylcytidine), 5-alkyluridine (e.g., ribothymidine), 5-halouridine (e.g., 5-bromouridine), 6-azapyrimidine, 6-alkylpyrimidine (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidineN4-ethanocytosine, 8-hydroxy-N6-methyladenine, 4-acetylcytosine, 5-fluorouracil; 5-bromouracil, 5-carboxymethylaminomethyl-2-thiouracil, 5 carboxymethylaminomethyl uracil, dihydrouracil, N6-isopentyl-adenine, 1-methylpseudouracil, 1-methylguanine, 2,2-dimethylguanine, 2-methylguanine, 3-methylcytosine, N6-methyladenine, 5-methoxy amino methyl-2-thiouracil, β-D-mannosylqueosine, 5-methoxycarbonylmethyluracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,-diaminopurine, methylpsuedouracil, 1-methylguanine, 1-methylcytosine.

In another aspect, the antisense oligonucleotide, siRNA or shRNA of the present invention comprises an abasic nucleotide. The term “abasic” as used herein, refers to moieties lacking a base or having other chemical groups in place of a base at the 1′ position, for example a 3′,3′-linked or 5′,5′-linked deoxyabasic ribose derivative. As used herein, a nucleotide with ‘modified base’ does not include an abasic nucleotide.

Modifications to Sugar Moiety.

Another secondary aspect relates to modifications to a sugar moiety. One or more nucleotides of the antisense oligonucleotide, siRNA or shRNA of the present invention may comprise a modified ribose moiety.

Modifications at the 2′-position wherein the 2′-OH is substituted include the non-limiting examples selected from alkyl, substituted alkyl, alkaryl-, aralkyl-, —F, —Cl, —Br, CN, —CF3, —OCF3, —OCN, —O-alkyl, —S-alkyl, —O-allyl, —S-allyl, HS-alkyl-O, —O-alkenyl, —S-alkenyl, —N-alkenyl, —SO-alkyl, -alkyl-OSH, -alkyl-OH, —O-alkyl-OH, —O-alkyl-SH, —S-alkyl-OH, —S-alkyl-SH, -alkyl-5-alkyl, -alkyl-O-alkyl, —ONO2, —NO2, —N3, —NH2, alkylamino, dialkylamino-, aminoalkyl-, aminoalkoxy, aminoacid, aminoacyl-, —ONH2, —O-aminoalkyl, —O-aminoacid, —O-aminoacyl, heterocycloalkyl-, heterocycloalkaryl-, aminoalkylamino-, polyalklylamino-, substituted silyl-, methoxyethyl- (MOE), alkenyl and alkynyl. “Locked” nucleic acids (LNA) in which the 2′ hydroxyl is connected, e.g., by a methylene bridge, to the 4′ carbon of the same ribose sugar is further included as a 2′ modification of the present invention. Preferred substituents are 2′-methoxyethyl, 2′-OCH3,2′-O-allyl, 2′-C-allyl, and 2′-fluoro.

In one embodiment, the siRNA of the present invention comprises 2′-OCH3 modifications at nucleotides 3, 5, 7, 9, 11, 13, 15 and 17 on the antisense strand and nucleotides 4, 6, 8, 10, 12, 14 and 16 on the sense strand, wherein said antisense strand is numbered from 5′-3′ and said sense strand is numbered from 3′-5′.

In one embodiment, the siRNA of the present invention comprises 2′-OCH3 modifications at nucleotides 7, 9, 11 and 13 on the antisense strand and nucleotides 8, and 12 on the sense strand, wherein said antisense strand is numbered from 5′-3′ and said sense strand is numbered from 3′-5′.

In one embodiment, the siRNA of the present invention comprises 2′-OCH3 modifications at nucleotides 7, 9 and 11 on the antisense strand and nucleotides 8, 10 and 12 on the sense strand, wherein said antisense strand is numbered from 5′-3′ and said sense strand is numbered from 3′-5′.

In another embodiment the antisense strand comprises 1, 2, 3, 4, 5, 6, 7, 8, 9, 10, 11, 12, 13, 14, 15, 16, 17, 18, 19, 20, 21, 22, 23, 24 or 25 2′-deoxy nucleotides.

In another embodiment the sense strand comprises 1, 2, 3, 4, 5, 6, 7, 8, 9, 10, 11, 12, 13, 14, 15, 16, 17, 18, 19, 20, 21, 22, 23, 24 or 25 2′-deoxy nucleotides.

In another embodiment the antisense strand comprises 1, 2, 3, 4, 5, 6, 7, 8, 9, 10, 11, 12, 13, 14, 15, 16, 17, 18, 19, 20, 21, 22, 23, 24 or 25 2′-fluoro nucleotides.

In another embodiment the sense strand comprises 1, 2, 3, 4, 5, 6, 7, 8, 9, 10, 11, 12, 13, 14, 15, 16, 17, 18, 19, 20, 21, 22, 23, 24 or 25 2′-fluoro nucleotides.

In another embodiment the pyrimidine nucleotides in the antisense strand are 2′-O-methylpyrimidine nucleotides. In another embodiment of the purine nucleotides in the antisense strand are 2′-O-methyl purine nucleotides. In another embodiment the pyrimidine nucleotides in the antisense strand are 2′-deoxy pyrimidine nucleotides. In another embodiment the purine nucleotides in the antisense strand are 2′-deoxy purine nucleotides. In another embodiment the pyrimidine nucleotides in the antisense strand are 2′-fluoro pyrimidine nucleotides. In another embodiment the purine nucleotides in the antisense strand are 2′-fluoro purine nucleotides. In another embodiment the pyrimidine nucleotides in the sense strand are 2′-O-methylpyrimidine nucleotides. In another embodiment of the purine nucleotides in the sense strand are 2′-O-methyl purine nucleotides. In another embodiment the pyrimidine nucleotides in the sense strand are 2′-deoxy pyrimidine nucleotides. In another embodiment the purine nucleotides in the sense strand are 2′-deoxy purine nucleotides. In another embodiment the pyrimidine nucleotides in the sense strand are 2′-fluoro pyrimidine nucleotides. In another embodiment the purine nucleotides in the sense strand are 2′-fluoro purine nucleotides. In another embodiment the pyrimidine nucleotides in the antisense duplex region are 2′-O-methylpyrimidine nucleotides. In another embodiment of the purine nucleotides in the antisense duplex region are 2′-O-methyl purine nucleotides. In another embodiment the pyrimidine nucleotides in the antisense duplex region are 2′-deoxy pyrimidine nucleotides. In another embodiment the purine nucleotides in the antisense duplex region are 2′-deoxy purine nucleotides. In another embodiment the pyrimidine nucleotides in the antisense duplex region are 2′-fluoro pyrimidine nucleotides. In another embodiment the purine nucleotides in the antisense duplex region are 2′-fluoro purine nucleotides. In another embodiment the pyrimidine nucleotides in the sense duplex region are 2′-O-methylpyrimidine nucleotides. In another embodiment of the purine nucleotides in the sense duplex region are 2′-O-methyl purine nucleotides. In another embodiment the pyrimidine nucleotides in the sense duplex region are 2′-deoxy pyrimidine nucleotides. In another embodiment the purine nucleotides in the sense duplex region are 2′-deoxy purine nucleotides. In another embodiment the pyrimidine nucleotides in the sense duplex region are 2′-fluoro pyrimidine nucleotides. In another embodiment the purine nucleotides in the sense duplex region are 2′-fluoro purine nucleotides. In another embodiment the pyrimidine nucleotides in the antisense duplex flanking regions are 2′-O-methylpyrimidine nucleotides. In another embodiment of the purine nucleotides in the antisense duplex flanking regions are 2′-O-methyl purine nucleotides. In another embodiment the pyrimidine nucleotides in the antisense duplex flanking regions are 2′-deoxy pyrimidine nucleotides. In another embodiment the purine nucleotides in the antisense duplex flanking regions are 2′-deoxy purine nucleotides. In another embodiment the pyrimidine nucleotides in the antisense duplex flanking regions are 2′-fluoro pyrimidine nucleotides. In another embodiment the purine nucleotides in the antisense duplex flanking regions are 2′-fluoro purine nucleotides. In another embodiment the pyrimidine nucleotides in the sense duplex flanking regions are 2′-O-methylpyrimidinenucleotides. In another embodiment of the purine nucleotides in the sense duplex flanking regions are 2′-O-methyl purine nucleotides. In another embodiment the pyrimidine nucleotides in the sense duplex flanking regions are 2′-deoxy pyrimidine nucleotides. In another embodiment the purine nucleotides in the sense duplex flanking regions are 2′-deoxy purine nucleotides. In another embodiment the pyrimidine nucleotides in the sense duplex flanking regions are 2′-fluoro pyrimidine nucleotides. In another embodiment the purine nucleotides in the sense duplex flanking regions are 2′-fluoro purine nucleotides.

Modifications to Phosphate Backbone:

Another secondary aspect relates to modifications to a phosphate backbone All or a portion of the nucleotides of a nucleic acid of the invention may be linked through phosphodiester bonds, as found in unmodified nucleic acid. A nucleic acid of the present invention however, may comprise a modified phosphodiester linkage. The phosphodiester linkages of an antisense oligonucleotide or either the antisense stand or the sense strand of an siRNA may be modified to independently include at least one heteroatom selected from the group consisting of nitrogen and sulfur. In one embodiment, a phosphoester group connecting a ribonucleotide to an adjacent ribonucleotide is replaced by a modified group. In one embodiment, one or more phosphoester group(s) connecting a ribonucleotide to an adjacent ribonucleotide is replaced by a phosphorothioate, alkylphosphonate, phosphorodithioate, phosphate ester, alkylphosphonothioate, phosphoramidate, carbamate, phosphate triester, acetamidate, peptide, or a carboxymethyl ester. In one embodiment, the modified group replacing the phosphoester group is selected from a phosphothioate, methylphosphonate or phosphoramidate group. In one embodiment, the modified group replacing the phosphoester group is selected from a phosphothioate, methylphosphonate or phosphoramidate group. In one embodiment, all of the nucleotides of the antisense oligonucleotide or antisense strand of an siRNA are linked through phosphodiester bonds. In another embodiment, all of the nucleotides of the antisense duplex region of an siRNA are linked through phosphodiester bonds. In another embodiment, all of the nucleotides of the sense strand of an siRNA are linked through phosphodiester bonds. In another embodiment, all of the nucleotides of the sense duplex region of an siRNA are linked through phosphodiester bonds. In another embodiment, the antisense oligonucleotide or antisense strand of an siRNA comprises 1, 2, 3, 4, 5, 6, 7, 8, 9 or 10 modified phosphoester groups. In another embodiment, the antisense duplex region of an siRNA comprises 1, 2, 3, 4, 5, 6, 7, 8, 9 or 10 modified phosphoester groups. In another embodiment, the sense strand of an siRNA comprises 1, 2, 3, 4, 5, 6, 7, 8, 9 or 10 modified phosphoester groups. In another embodiment, the sense duplex region of an siRNA comprises 1, 2, 3, 4, 5, 6, 7, 8, 9 or 10 modified phosphoester groups.

5′ and 3′ end Modifications:

Another secondary aspect relates to 5′ and 3′ modifications. The nucleic acid of the present invention may include nucleic acid molecules comprising one or more modified nucleotides, abasic nucleotides, acyclic or deoxyribonucleotide at the terminal 5′- or 3′-end of an antisense oligonucleotide or on either or both of the sense or antisense strands of an siRNA.

In one embodiment, the 5′- and 3′-end nucleotides of an antisense oligonucleotide or both the sense and antisense strands of a siRNA are unmodified. In another embodiment, the 5′-end nucleotide of the sense strand of a siRNA is modified. In another embodiment, the 3′-end nucleotide of the antisense strand of a siRNA is modified. In another embodiment, the 3′-end nucleotide of the sense strand of a siRNA is modified. In another embodiment, the 3′-end nucleotide of the antisense strand of a siRNA and the 3′-end nucleotide of the sense strand of a siRNA are modified. In another embodiment, the 3′-end nucleotide of the antisense strand of a siRNA and the 5′-end nucleotide of the sense strand of a siRNA are modified. In another embodiment, the 3′-end nucleotide of the antisense strand of a siRNA and both the 5′- and 3′-end nucleotides of the sense strand of a siRNA are modified.

In another embodiment, the 5′-end nucleotide of an antisense oligonucletide or an antisense strand of a siRNA is phosphorylated. In another embodiment, the 5′-end nucleotide of the sense strand of a siRNA is phosphorylated. In another embodiment, the 5′-end nucleotides of both the antisense strand and the sense strand of a siRNA are phosphorylated. In another embodiment, the 5′-end nucleotide of the antisense strand of a siRNA is phosphorylated and the 5′-end nucleotide of the sense strand has a free hydroxyl group (5′-OH). In another embodiment, the 5′-end nucleotide of the antisense strand of a siRNA is phosphorylated and the 5′-end nucleotide of the sense strand is modified.

Modifications to the 5′- and 3′-end nucleotides are not limited to the 5′ and 3′ positions on these terminal nucleotides. Examples of modifications to end nucleotides include, but are not limited to, biotin, inverted (deoxy) abasics, amino, fluoro, chloro, bromo, CN, CF, methoxy, imidazole, carboxylate, thioate, C₁ to C₁₀ lower alkyl, substituted lower alkyl, alkaryl or aralkyl, OCF₃, OCN, O-, S-, or N-alkyl; O-, S-, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described, e.g., in PCT patent application WO 99/54459, European patents EP 0 586 520 B1 or EP 0 618 925 B1, incorporated by reference in their entireties. As used herein, “alkyl” means C₁-C₁₂-alkyl and “lower alkyl” means C₁-C₆-alkyl, including C₁-, C₂-, C₃-, C₄-, C₅- and C₆-alkyl.

In another aspect, the 5′-end of an antisense oligonucleotide, the 5′-end of an antisense strand, the 5′-end of the sense strand, the 3′-end of the antisense oligonucleotide, the 3′-end of the antisense strand or the 3′-end of the sense strand is covalently connected to a prodrug moiety. In one embodiment, the moiety is cleaved in an endosome. In another the moiety is cleaved in the cytoplasm.

In another embodiment the terminal 3′ nucleotide or two terminal 3′-nucleotides on either or both of the antisense strand or sense strand is a 2′-deoxynucleotide. In another embodiment the 2′-deoxynucleotide is a 2′-deoxy-pyrimidine. In another embodiment the 2′-deoxynucleotide is a 2′ deoxy-thymidine. In another embodiment the terminal 3′ nucleotide or two terminal 3′-nucleotides on either or both of the antisense strand or sense strand are not base paired, i.e., they are one or two nucleotide overhangs. In one embodiment the 3′ end of both antisense and sense strand have a —TT dinucleotide overhang.

On aspect of the present invention relates to modifications of an antisense oligonucleotide to form a gapmer. A “gapmer” is defined as an antisense oligonucleotide having a 2′-deoxyoligonucleotide region flanked by non-deoxyoligonucleotide segments. The central region is referred to as the “gap.” The flanking segments are referred to as “wings.” Each wing can be one or more non-deoxyoligonucleotide monomers. In one embodiment, the gapmer is a ten deoxynucleotide gap flanked by five non-deoxynucleotide wings. This is referred to as a 5-10-5 gapmer. Other configurations are readily recognized by those skilled in the art. In one embodiment the wings comprise 2′-O-(2-methoxyethyl) (2′-MOE) modified nucleotides. In another embodiment the gapmer has a phosphorothioate backbone. In another embodiment the gapmer has 2′-MOE wings and a phosphorothioate backbone. Other suitable modifications are readily recognizable by those skilled in the art.

shRNA and Linked siRNA:

Another aspect relates to shRNA and linked siRNA. It is within the present invention that the double-stranded structure is formed by two separate strands, i.e. the antisense strand and the sense strand. However, it is also within the present invention that the antisense strand and the sense strand are covalently linked to each other. Such linkage may occur between any of the nucleotides forming the antisense strand and sense strand, respectively. Such linkage can be formed by covalent or non-covalent linkages. Covalent linkage may be formed by linking both strands one or several times and at one or several positions, respectively, by a compound preferably selected from the group comprising methylene blue and bifunctinoal groups. Such bifunctional groups are preferably selected from the group comprising bis(2-chloroethyl)amine, N-acetyl)-N′-(p-glyoxylbenzoyl)cystamine, 4-thiouracile and psoralene.

In one aspect, the antisense strand and the sense strand of an siRNA of the invention are linked by a loop structure. In one embodiment, the loop structure is comprised of a non-nucleic acid polymer. In another embodiment, the non-nucleic acid polymer is polyethylene glycol. In another embodiment, the 5′-end of the antisense strand is linked to the 3′-terminus of the sense strand. In another embodiment, the 3% end of the antisense strand is linked to the 5′-end of the sense strand.

In another embodiment, the antisense strand and the sense strand of an siRNA of the invention are linked by a loop consists of a nucleic acid. As used herein, locked nucleic acid (LNA) (Elayadi and Corey (2001) Curr Opin lnvestig Drugs. 2(4):558-61) and peptide nucleic acid (PNA) (reviewed in Faseb J. (2000) 14:1041-1060) are regarded as nucleic acids and may also be used as loop forming polymers. In one embodiment the nucleic acid is ribonucleic acid. In one embodiment the nucleic acid is deoxyribonucleic acid. In one embodiment, the 5′-end of the antisense strand of an siRNA is linked to the 3′-terminus of the sense strand of the siRNA to form an shRNA. In another embodiment, the 3′-end of the antisense strand of an siRNA is linked to the 5% end of the sense strand of the siRNA to form a shRNA. The loop consists of a minimum length of four nucleotides or nucleotide analogues. In certain embodiments the loop consists of 4, 5, 6, 7, 8, 9, 10, 11, 12, 13, 14 or 15 nucleotides or nucleotide analogs. In one embodiment the loop nucleotide sequence is a portion of the antisense strand. In another embodiment the loop nucleotide sequences is a portion of the sense strand. In another embodiment, a portion of both the antisense stand and the sense strand form the loop nucleotide sequence. In another embodiment the loop nucleotide sequences is a heterologous sequence, i.e., not the same as or complementary to the target sequence.

The ribonucleic acid constructs may be incorporated into suitable expression vector systems. Preferably the vector comprises a promoter for the expression of RNAi. Preferably the respective promoter is pol III and more preferably the promoters are the U6, H1, 7SK promoter as described in Good et al. (1997) Gene Ther, 4, 45-54.

Processes of Making:

The nucleic acid of the present invention can be produced using routine methods in the art including chemically synthesis or expressing the nucleic acid either in vitro (e.g., run off transcription) or in vivo. In one embodiment, the antisense oligonucleotide or siRNA is produced using solid phase chemical synthesis. In another embodiment, the nucleic acid is produced using an expression vector. In one embodiment, the expression vector produced the nucleic acid of the invention in the target cell. Accordingly, such vector can be used for the manufacture of a medicament. Methods for the synthesis of the nucleic acid molecule described herein are known to the ones skilled in the art.

In one embodiment said siRNA or shRNA is part of an expression vector adapted for eukaryotic expression; preferably said siRNA or shRNA is operably linked to at least one promoter sequence.

In another embodiment the invention said cassette is provided with at least two promoters that transcribe both sense and antisense strands of said nucleic acid molecule.

In another embodiment of the invention said cassette comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts thereby forming an shRNA.

“Promoter” is an art recognised term and, for the sake of clarity, includes the following features which are provided by example only. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors which have been shown to bind specifically to enhancer elements. The binding/activity of transcription factors (please see Eukaryotic Transcription Factors, by David S Latchman, Academic Press Ltd, San Diego) is responsive to a number of physiological/environmental cues.

Promoter elements also include so called TATA box and RNA polymerase initiation selection sequences which function to select a site of transcription initiation. These sequences also bind polypeptides which function, inter alia, to facilitate transcription initiation selection by RNA polymerase.

Adaptations also include the provision of selectable markers and autonomous replication sequences which facilitate the maintenance of said vector in either the eukaryotic cell or prokaryotic host. Vectors which are maintained autonomously are referred to as episomal vectors.

Adaptations which facilitate the expression of vector encoded genes include the provision of transcription termination/polyadenylation sequences. Expression control sequences also include so-called Locus Control Regions (LCRs). These are regulatory elements which confer position-independent, copy number-dependent expression to linked genes when assayed as transgenic constructs. LCRs include regulatory elements that insulate transgenes from the silencing effects of adjacent heterochromatin, Grosveld et al., Cell (1987), 51: 975-985.

There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, NY and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

The use of viruses or “viral vectors” as therapeutic agents is well known in the art. Additionally, a number of viruses are commonly used as vectors for the delivery of exogenous genes. Commonly employed vectors include recombinantly modified enveloped or non-enveloped DNA and RNA viruses, preferably selected from retroviridae baculoviridiae, parvoviridiae, picornoviridiae, herpesveridiae, poxyiridae, adenoviridiae, or picornnaviridiae. Chimeric vectors may also be employed which exploit advantageous elements of each of the parent vector properties (See e.g., Feng, et al. (1997) Nature Biotechnology 15:866-870). Such viral vectors may be wild-type or may be modified by recombinant DNA techniques to be replication deficient, conditionally replicating or replication competent.

Preferred vectors include those derived from retroviral genomes (e.g. lentivirus) and adeno-associated virus. Viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein a gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al.; U.S. Pat. No. 5,871,726 issued Feb. 16, 1999 the entire teachings of which are herein incorporated by reference.

Additionally, the viral genome may be modified to include inducible promoters which achieve replication or expression only under certain conditions. Examples of inducible promoters are known in the scientific literature (See, e.g. Yoshida and Hamada (1997) Biochem. Biophys. Res. Comm. 230:426-430; lida, et al. (1996) J. Virol. 70(9):6054-6059; Hwang, et al. (1997) J. Virol 71(9):7128-7131; Lee, et al. (1997) Mol. Cell. Biol. 17(9):5097-5105; and Dreher, et al. (1997) J. Biol. Chem. 272(46); 29364-29371.

In one embodiment said vectors include promoters that are substantially lung or cancer specific; preferably said promoters are preferentially active in lung cancer cells.

Delivery/Formulations:

Antisense oligonucleotides and siRNA can be delivered to cells, both in vitro and in vivo, by a variety of methods known to those of skill in the art, including direct contact with cells (“naked” delivery) or by in combination with one or more agents that facilitate targeting or delivery into cells. Such agents and methods include lipoplexes, liposomes, iontophoresis, hydrogels, cyclodextrins, nanocapsules, micro- and nanospheres and proteinaccous vectors (e.g., Bioconjugate Chem. (1999) 10:1068-1074 and WO 00/53722).

A nucleic acid composition may be delivered in vivo either locally or systemically by various means including intravenous, subcutaneous, intramuscular or intradermal injection or inhalation.

The molecules of the instant invention can be used as pharmaceutical agents. Preferably, pharmaceutical agents prevent, modulate the occurrence, or treat (alleviate a symptom to some extent, preferably all of the symptoms) of a disease state in a subject. In the case of treating cancer, the treatment reduces tumor burden or tumor mass in the subject.

There is also provided the use of a composition comprising surface-modified liposomes containing poly (ethylene glycol) lipids (PEG-modified, or long-circulating liposomes or stealth liposomes). These formulations offer a method for increasing stability of a liposome or lipoplex solutions by preventing their aggregation and fusion. The formulations also have the added benefit in vivo of resisting opsonization and elimination by the mononuclear phagocytic system (MPS or RES), thereby enabling longer blood circulation times and enhanced tissue exposure for the encapsulated drug. Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42,24864-24780; Choi et al., Internaional PCT Publication No. WO 96/10391; AnselI et al., International PCT Publication No. WO 96/10390; Holland et al., International PCT Publication No. WO 96/10392). Long-circulating liposomes also protect the siRNA from nuclease degradation.

The nucleic acid of the present invention may be formulated as pharmaceutical compositions. The pharmaceutical compositions may be used as medicaments or as diagnostic agents, alone or in combination with other agents. For example, one or more nucleic acid of the invention can be combined with a delivery vehicle (e.g., liposomes) and/or excipients, such as carriers, diluents. The term “excipient” refers to a pharmaceutically acceptable, pharmaceutically inactive substance used as a carrier for the pharmaceutically active ingredient(s). Methods for the delivery of nucleic acid molecules are known in the art and described, e.g., in Akhtar et al., 1992, Trends Cell Bio., 2, 139; Delivery Strategies for Antisense Oligonucleotide Therapeutics, ed. Akhtar, 1995, Maurer et al., 1999, Mol. Memb. Biol., 16, 129-140; Hofland and Huang, 1999, Handb. Exp. Pharmacol., 137, 165-192; and Lee et al., 2000, ACS Symp. Ser., 752, 184-192, U.S. Pat. No. 6,395,713 and PCT WO 94/02595 (each of which are incorporated herein by reference in their entireties). The nucleic acid of the present invention can also be administered in combination with other therapeutic compounds, either administrated separately or simultaneously, e.g., as a combined unit dose. In one embodiment, the invention includes a pharmaceutical composition comprising one or more nucleic acid according to the present invention in a physiologically/pharmaceutically acceptable excipient, such as a stabilizer, preservative, diluent, buffer, and the like.

An example of delivery formulations suitable to deliver the nucleic acids of the present invention include those disclosed in WO28137758 (US28317839A1) and WO29046220A2, each incorporated by reference in its entirety.

Suitable delivery systems include the PTD-DRBD of Traversa Therapeutics, San Diego, Calif., which comprises a Peptide Transduction Doman (PTD) fused to a Double-stranded RNA Binding Doman (DRBD) The PTD (also called a cell penetrating peptide or CPP) is a peptide that binds proteoglycans on the cell surface. Bound PTD is taken up into cells by macropinocytosis, a specialized form of fluid phase uptake that all cells perform. An advantage of macropinocytosis is that it does not involve the lysosomal pathway, thereby avoiding the need for the siRNA payload to escape the endosomes. The DRBD is self explanatory, i.e., a binding domain of a protein that binds double stranded RNA. The PTD-DRBD is disclosed in WO2007095152 (US20090093026A1) (assigned to The Regents Of The University Of California) and published in Nature Biotechnology (2009) 27(6): 567-571 (each patent application and publication are incorporated by reference in its entirety).

In one embodiment the PTD is a portion of the HIV-1 tat protein (RKKRRQRRR) repeated three times. In one embodiment the DRBD comprises the 65 amino acid (FFMEELNTYRQKQGWLKYQELPNSGPPHDRRFTFQVIIDG REFPEGEGRSKKEAKNAAAKLAVEILNKE) portion of the Protein Kinase RNA-activated or PKR protein (also known as eukaryotic translation initiation factor 2-alpha kinase 2 (EIF2AK2) and PRKR). In other embodiments the PTD is a herpes viral VP22 protein; a polypeptide comprising a human immunodeficiency virus (HIV) TAT protein; a polypeptide comprising a homeodomain of an Antennapedia protein (Antp HD), and functional fragments thereof. In other embodiments the DRBD comprises a sequence selected from the group consisting of histone, RDE-4 protein, protamine, dsRNA binding proteins (Accession numbers in parenthesis) include: PKR (AAA36409, AAA61926, Q03963), TRBP (P97473, AAA36765), PACT (AAC25672, AAA49947, NP609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP499172, NP198700, BAB19354), HYLL (NP563850), hyponastic leaves (CAC05659, BAB00641), ADAR1 (AAB97118, P55266, AAK16102, AAB51687, AF051275), ADAR2P78563, P51400, AAK17102, AAF63702), ADAR3 (AAF78094, AAB41862, AAF76894), TENR (XP059592, CAA59168), RNaselll (AAF80558, AAF59169, Z81070002555/S55784, P05797), and Dicer (BAA78691, AF408-401, AAF56056, S44849, AAF03534, Q9884), RDE-4 (AY071926), FLJ20399 (NP060273, BAB26260), CG1434 (AAF48360, EAA12065, CAA21662), CG13139 (XP059208, XP143416, XP110450, AAF52926, EEA14824), DGCRK6 (BAB83032, XP110167) CG1800 (AAF57175, EAA08039), FLJ20036 (AAH22270, XP134159), MRP-L45 (BAB14234, XP129893), CG2109 (AAF52025), CG12493 (NP647927), CG10630 (AAF50777), CG17686 (AAD50502), T22A3.5 (CAB03384) and Accession number EAA14308.

Another suitable delivery system is Clycodextrin Based Delivery of Calando Pharmaceuticals (formerly Insert Therapeutics and subsidiary of the holding company Arrowhead Research Corporation) referred to as RNAi/Oligonucleotide Nanoparticle Delivery (RONDEL) technology.

The linear cyclodextrins of RONDEL are co-polymers formed by linking the cyclic oligosaccharides with a cation containing chemical linking group. Amines and imidazoles found in the linking and termini groups aid in endosomal release. The polymers, called cyclodextrin-containing polycations (CDP) condense with the siRNA payload.

The inner ring or core of the cyclodextrin molecules are hydrophobic and can be used to incorporate hydrophobic compounds. The complexes formed are called inclusion complexes. In the RONDEL formulation, the hydrophobic cores of cyclodextrins subunits are used to anchor molecules of adamantine-PEG conjugates. PEG is conjugated to the adamantine and then the PEG-adamantane conjugates are combined with linear cyclodextrins (CDP). To target the nanoparticles to a particular cell type, a ligand in conjugated onto the PEG portion of the PEG-adamantane molecules, forming an adamantine-PEG-ligand conjugate. In the case of RONDEL, human transferrin protein (Tf) is one example of a ligand that can be used because most cancer cells overexpress the human transferrin receptor on the cell surface.

An example of a RONDEL formluation is disclosed in David et al. (2010) Nature 464:1067-1070 and Heidel et al. PNAS (2007) 104(14):5717-5721 (each incorporate by reference in its entiry). The linear cyclodextrin technology is disclosed and claimed in WO0001734 (US20070025952A1, US20020151523A1, US7091192, US6884789 and US6509323) (each incorporated by reference in its entirety). Linear cyclodextrin inclusion complexes, including inclusion complexes with adamantine-PEG and adamantine-PEG-TF is disclosed in WO0249676 (US20070128167A1, US20060182795A1, US20030017972A1, US20030008818A1, US7166302 and US7018609) (each incorporated by reference in its entirety). Other formulations include SNALPs, disclosed in Nature Biotechnology (2005) 23(8):1002-1007, incorporated reference in its entierty. SNALPs formulations are disclosed in WO05120152 (US20060083780A1 & US20060008910A1), WO05026372A1 (US20050175682A1, WO06007712 (US20060051405A1 &US20060025366A1), each incorporated by reference in its entirety. An example of SNALP formulation is 1,2-distearoyl-sn-glycero-3phosphocholine (DSPC) MW 387, cholesterol MW 790, 1,2-dilinoleyloxy-N,N-dimethyl-3-aminopropane (DLinDMA) MW 616 and 3-N-[ω-methoxy poly(ethylene glycol)_{average MW 2000})carbamoyl]-1,2-dimyristyloxy-propylamine (PEG-C-DMA) MW 2524. In another useful embodiment, the DLinDMA component above is replaced with 2,2-dilinoleyl-4-(2-dimethylaminoethyl)[1,3]-dioxolane (DLin-KC2-DMA). This and other useful formulations are disclosed in WO2009086558 and WO2009132131, each incorporated by reference in its entirety.

Other useful formualtion are based on lipidoids as disclosed in Nature Biotechnology (2008) 26:561-569 and Love et al. PNAS (2010) 107(5):1864-1869 and WO28042973A2 (USUS20090023673A1) and WO28042973A2 (USUS20090023673A1), each incorporated by reference in its entirety.

Other useful formuations are those disclosed in WO2010021865 and WO2007086881A2 and WO2007086883 (US20100063308A1 US20090048197A1 US20080188675A1 US20080020058A1 US20060240554A1 U.S. Pat. No. 7,691,405, U.S. Pat. No. 7,641,915, U.S. Pat. No. 7,514,099 and U.S. Pat. No. 7,404,969) of WO2008147438A2 (US20100048888A1, US20090048197A1, US20080020058A1 and U.S. Pat. No. 7,691,405, U.S. Pat. No. 7,404,969), each incorporated by reference in its entirety.

Other useful formluation include those of WO2007095152 (US20090093026A1) and in Nature Biotechnology (2009) 27(6): 567-571, each incorporated by reference in its entirety.

Other useful formulations include those from Rozema et al. (2007) PNAS 104(32):12982-12987, Heidel et al. PNAS (2007) 104(14):5717-5721), Wakefield et al. (2005) Bioconjugate Chem. 16 (5):1204-1208 and WO0001734 (US20070025952A1, US20020151523A1, U.S. Pat. No. 7,091,192, U.S. Pat. No. 6,884,789 and U.S. Pat. No. 6,509,323). WO0249676 (US20070128167A1, US20060182795A1, US20030017972A1, US20030008818A1, U.S. Pat. No. 7,166,302 and U.S. Pat. No. 7,018,609, WO04090107 (US20070105804A1, US20070036865A1 and US20040198687A1), WO2008022309 (US20090048410A1, US20090023890A1, US20080287630A1, US20080287628A1, US20080281074A1, US20080281044A1, US20080281041A1, US20080269450A1, US20080152661A1), each incorporated by reference in its entirety.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of an agent according to the invention for producing the desired response in a unit of weight or volume suitable for administration to a patient.

The doses of the siRNA/shRNA according to the invention administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Dosage levels for the medicament and pharmaceutical compositions of the invention can be determined by those skilled in the art by routine experimentation. In one embodiment, a unit dose contains between about 0.01 mg/kg and about 100 mg/kg body weight of nucleic acid. In one embodiment, the dose of nucleic acid is about 10 mg/kg and about 25 mg/kg body weight. In one embodiment, the dose of nucleic acid is about 1 mg/kg and about 10 mg/kg body weight. In one embodiment, the dose of nucleic acid is about 0.05 mg/kg and about 5 mg/kg body weight. In another embodiment, the dose of nucleic acid is about 0.1 mg/kg and about 5 mg/kg body weight. In another embodiment, the dose of nucleic acid is about 0.1 mg/kg and about 1 mg/kg body weight. In another embodiment, the dose of nucleic acid is about 0.1 mg/kg and about 0.5 mg/kg body weight. In another embodiment, the dose of nucleic acid is about 0.5 mg/kg and about 1 mg/kg body weight. In another embodiment doses of siRNA/shRNA are between 1 nM-1 μM. In certain embodiments doses can range from 1 nM-500 nM, 5 nM-200 nM, and 10 nM-100 nM. Other protocols for the administration of compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents'.The pharmaceutical compositions may conveniently be presented in unit dosage form and may be prepared by any of the methods well-known in the art of pharmacy.

In one embodiment the pharmaceutical compositions is a sterile aqueous suspension or solution. In another embodiment the pharmaceutical compositions is a sterile injectable aqueous suspension or solution. In one embodiment the pharmaceutical composition is in lyophilized form. In one embodiment, the pharmaceutical composition comprises lyophilized lipoplexes, wherein the lipoplexes comprises a nucleic acid of the present invention. In another embodiment, the pharmaceutical composition comprises an aqueous suspension of lipoplexes, wherein the lipoplexes comprises a nucleic acid of the present invention.

The pharmaceutical compositions and medicaments of the present invention may be administered to mammal. In one embodiment, the mammal is selected from the group consisting humans, dogs, cats, horses, cattle, pig, goat, sheep, mouse, rat, hamster and guinea pig. In one embodiment, the mammal is a human. In another embodiment, the mammal is a non-human mammal.

As used herein, the term “cancer” or “cancerous” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “cancer recurrence” as used herein refers to the detection or return of cancer after a period when no cancer cells could be detected in the body. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. Further examples include lung cancer for example small cell lung carcinoma or a non-small cell lung cancer. Other classes of lung cancer include neuroendocrine cancer, sarcoma and metastatic cancers of different tissue origin.

According to another aspect of the invention there is provided a method of diagnosing cancer in a subject comprising:
i) providing an isolated biological sample to be tested;
ii) determining whether a Ciz1 b-variant transcript is present in said biological sample,
   wherein the presence of said Ciz1 b-variant transcript indicates the presence of cancer in said subject.

In one embodiment the subject is a human.

In one embodiment the cancer is a cancer recurrence.

Ciz1 b-variant has been detected in several cancers including lung cancer (both NSCLC and SCLC), breast cancer, thyroid cancer, bladder cancer, liver cancer, kidney cancer, lymphomas and leukemias. In one embodiment the cancer is lung cancer. In a further embodiment the lung cancer is NSCLC. In another further embodiment the lung cancer is SCLC. In another embodiment the cancer is breast cancer. In another embodiment the cancer is thyroid cancer. In a further embodiment the thyroid cancer is medullary thyroid cancer. In another further embodiment the thyroid cancer is Hurthle cell carcinoma. In another further embodiment the thyroid cancer is papillary thyroid cancer. In another further embodiment the thyroid cancer is follicular thyroid cancer. In another embodiment the cancer is a lymphoma. In a further embodiment the lymphoma is B cell lymphoma. In another further embodiment the lymphoma is Hodgkin's lymphoma. In another further embodiment the lymphoma is diffuse large B-cell lymphoma. In another further embodiment the lymphoma is follicular lymphoma. In another further embodiment the lymphoma is anaplastic large cell lymphoma. In another further embodiment the lymphoma is extranodal marginal zone B-cell lymphoma. In another further embodiment the lymphoma is splenic marginal zone B-cell lymphoma. In another further embodiment the lymphoma is mantle cell lymphoma. In another embodiment the cancer is leukemia. In another further embodiment the leukemia is chronic lymphocytic leukemia. In another further embodiment the leukemia is hairy cell leukemia.

In one embodiment said biological sample is selected from: a solid tissue sample, blood, plasma, serum, sputum, urine or bronchoalveolar lavage. In a further embodiment the sample is a solid tissue sample. In another further embodiment the sample is blood. In another further embodiment the sample is plasma. In another further embodiment the sample is serum. In another further embodiment the sample is sputum. In another further embodiment the sample is urine. In another further embodiment the sample is bronchoalveolar lavage. In another further embodiment the biological sample is circulating tumor cells (CTCs). In a further embodiment the Ciz1 b-variant transcript in said biological sample is extracellular, i.e., present outside of a cell.

In one embodiment, the method uses polymerase chain reaction (PCR) to detect the presence of a Ciz1 b-variant transcript. In another embodiment, nucleotide primers are used in PCR to amplify a portion of nucleic acid that spans the junction between exon 14b and exon 15. In another embodiment a nucleic acid product amplified using PCR comprises the nucleotide sequence 5′ TGGACCTCACCTCGATCTCT 3′. In another embodiment a nucleic acid amplified using PCR comprises the nucleotide sequence 5′ GATATATCTCTGGACCTCACCTCGATCTCTTCTTCATCCT 3′. In another embodiment the amplified nucleic acid product with a normal matched control.

In one embodiment the cancer is a lymphoma, lung, breast, kidney, thyroid or colon cancer. In one embodiment the cancer is small cell lung cancer (SCLC). In another embodiment the cancer is non-small cell lung cancer. In another embodiment the cancer is breast cancer. In another embodiment the cancer is kidney cancer. In another embodiment the cancer is a lymphoma. In another embodiment the cancer is colon cancer.

In one embodiment said amplified products are digested with a restriction endonuclease that does not cleave the nucleic acid sequence 5′GAAGAAGAGATCGAGGTGAGGTCCAGAGA3′.

In another embodiment said restriction endonuclease is CAC81.

In another embodiment said oligonucleotide primer pair is adapted to specifically amplify a nucleic acid molecule comprising a nucleic acid sequence GAAGAAGAGATCGAGGTGAGGTCCAGAGA.

In another embodiment one of said oligonucleotide primers in said primer pair comprises or consists of the nucleic acid sequence:

5′ GAAGAGATCGAGGTGAGGTC 3′.

In another embodiment said oligonucleotide primer pairs comprise or consist of nucleic acid sequences:

5′ GAAGAGATCGAGGTGAGGTC 3′;
and
5′ GAAGAAGAGATCGAGGTGAGGTCCAGAGA 3′.

In another embodiment an amplified product containing the sequence GAAGAAGAGATCGAGGTGAGGTCCAGAGA is detected with an oligonucleotide probe comprising or consisting of the nucleotide sequence:

5′ TGGACCTCACCTCGATCTCTTCTTCA 3′.

In a preferred method of the invention said biological sample comprises lung cells.

In another embodiment said diagnosis is combined with a treatment regime suitable for the cancer diagnosed.

In another embodiment said treatment regime comprises the administration of an anti-cancer agent.

In another embodiment said chemotherapeutic agent is selected from the group consisting of: cisplatin, carboplatin, irinotecan, topotecan, camptothecin, etoposide, doxorubicin, paclitaxel, docetaxel, gemcitabine and vinorelbine.

In another embodiment said anti-cancer agent is a siRNA or shRNA according to the present invention.

In another embodiment said treatment regime comprises the administration of at least one siRNA or shRNA and the chemotherapeutic agent is administered separately, simultaneously or sequentially.

In one embodiment the cancer is lung cancer. In another embodiment said lung cancer is small cell lung carcinoma. In another embodiment said lung cancer is non-small cell lung cancer.

In one aspect of the invention there is provided a method of detecting the presence of a Ciz1 b-variant polypeptide translated from a Ciz1 b-variant mRNA in human with cancer, said method comprising the steps of:
i) providing an isolated biological sample to be tested;
ii) detecting the presence of said Ciz1 b-variant polypeptide.

In one embodiment the biological sample is plasma.

In one embodiment the cancer is lung cancer.

In one aspect of the invention there is provided a method to diagnose cancer in a subject by detecting the presence of a Ciz1 b-variant polypeptide translated from a Ciz1 b-variant mRNA, said method comprising the steps of:
iii) providing an isolated biological sample to be tested;
iv) detecting the presence of said a Ciz1 b-variant polypeptide, wherein the presence of said Ciz1 b-variant polypeptide is indicative of the presence of cancer.

In one embodiment the subject is a human.

In one embodiment the biological sample is plasma.

In one embodiment the cancer is a cancer recurrence.

In one embodiment the cancer is lung cancer.

In one embodiment of the invention there is provided a method to diagnose cancer in a subject by detecting the presence of a Ciz1 b-variant polypeptide translated from a Ciz1 b-variant mRNA, said method comprising the steps of:
i) providing an isolated biological sample to be tested;
ii) contacting said biological sample with an antibody or antigen binding fragment thereof that specifically binds said Ciz1 b-variant polypeptide;
iii) detecting the presence of said antibody or antigen binding fragment bound to said Ciz1 b-variant polypeptide, wherein the presence of said Ciz1 b-variant polypeptide is indicative of the presence of cancer.

In one embodiment the cancer is a cancer recurrence.

In one embodiment the subject is a human.

In one embodiment said antibody specifically binds to said Ciz1 b-variant polypeptide but does not specifically bind a Ciz1 polypeptide translated from a mRNA comprising exon 14a.

In one embodiment said biological sample is selected from: a solid tissue sample, blood, plasma, serum, sputum, urine or bronchoalveolar lavage. In a further embodiment the biological sample is a solid tissue sample. In another further embodiment the biological sample is blood. In another further embodiment the biological sample is plasma. In another further embodiment the biological sample is serum. In another further embodiment the biological sample is sputum. In another further embodiment the sample is urine. In another further embodiment the biological sample is bronchoalveolar lavage. In another further embodiment the biological sample is circulating tumor cells (CTCs). In a further embodiment the Ciz1 b-variant transcript in said biological sample is extracellular, i.e., present outside of a cell.

In one embodiment the cancer is lung cancer. In a further embodiment the lung cancer is NSCLC. In a further embodiment the lung cancer is stage 0 NSCLC. In a further embodiment the lung cancer is stage I NSCLC. In a further embodiment the lung cancer is stage II NSCLC. In a further embodiment the lung cancer is stage III NSCLC. In a further embodiment the lung cancer is stage IV NSCLC. In another further embodiment the lung cancer is SCLC. In another further embodiment the lung cancer is limited stage SCLC. In another further embodiment the lung cancer is extensive stage SCLC. In another embodiment the cancer is breast cancer. In another embodiment the cancer is thyroid cancer. In a further embodiment the thyroid cancer is medullary thyroid cancer. In another further embodiment the thyroid cancer is Hurthle cell carcinoma. In another further embodiment the thyroid cancer is papillary thyroid cancer. In another further embodiment the thyroid cancer is follicular thyroid cancer. In another embodiment the cancer is a lymphoma. In a further embodiment the lymphoma is B cell lymphoma. In another further embodiment the lymphoma is Hodgkin's lymphoma. In another further embodiment the lymphoma is diffuse large B-cell lymphoma. In another further embodiment the lymphoma is follicular lymphoma. In another further embodiment the lymphoma is anaplastic large cell lymphoma. In another further embodiment the lymphoma is extranodal marginal zone B-cell lymphoma. In another further embodiment the lymphoma is splenic marginal zone B-cell lymphoma. In another further embodiment the lymphoma is mantle cell lymphoma. In another embodiment the cancer is leukemia. In another further embodiment the leukemia is chronic lymphocytic leukemia. In another further embodiment the leukemia is hairy cell leukemia. In another further embodiment the cancer is renal cancer. In another further embodiment the cancer is liver cancer. In another further embodiment the cancer is bladder cancer.

In one embodiment said b-variant polypeptide is a proteolytically cleaved Ciz1 b-variant polypeptide fragment. In a further embodiment the polypeptide fragment comprises the polypeptide sequences encoded by exons 14b and 15. In a further embodiment the polypeptide fragment comprises the amino acid sequence DEEEIEVRSRDIS. In another embodiment said fragment migrates with an apparent molecular weight of between approximately 50-60 kDa on an 8% SDS-PAGE, depending on the degree of degradation. In a further embodiment said fragment migrates with an apparent molecular weight of approximately 50 kDa on an 8% SDS-PAGE. In one embodiment said antibody specifically binds to a contiguous epitope that includes amino acid residues encoded by both exon 14b and exon 15. In another embodiment said antibody specifically binds to a Ciz1 b-variant polypeptide but does not bind specifically bind a Ciz1 polypeptide translated from a mRNA comprising exon 14a. In another embodiment said antibody said antibody specifically binds to a Ciz1 b-variant polypeptide with an affinity at least 10 fold greater than to a Ciz1 polypeptide translated from a mRNA comprising exon 14a. In one embodiment said antibody binds with at least 100 fold greater affinity to a Ciz1 b-variant polypeptide as compared to a Ciz1 polypeptide translated from a mRNA comprising exon 14a. In one embodiment said antibody binds with at least 1,000 greater affinity to a Ciz1 b-variant polypeptide as compared to a Ciz1 polypeptide translated from a mRNA comprising exon 14a. In one embodiment said antibody binds with at least 10,000 greater affinity to a Ciz1 b-variant polypeptide as compared to a Ciz1 polypeptide translated from a mRNA comprising exon 14a. In one embodiment said antibody binds with at least 100,000 greater affinity to a Ciz1 b-variant polypeptide as compared to a Ciz1 polypeptide translated from a mRNA comprising exon 14a.

In one embodiment said antibody specifically binds to the amino acid sequence DEEEIEVRSRDIS. In one embodiment said antibody specifically binds to the amino acid sequence DEEEIEVRSRDIS but does not specifically bind to the either the amino acid sequence DEEEIE, VRSRDIS or DEEEIEVEEELCKQVRSRDIS. In one embodiment said antibody specifically binds to the amino acid sequence EGDEEEEEDDEDEEEIEVRSRDISREEWKGSETY but not the amino acid sequence EGDEEEEEDDEDEEEIEVEEELCKQVRSRDISREEWKGSETY. In another embodiment said antibody specifically binds to the amino acid sequence DEEEEEDDEDEEEIEVRSRDISREEWKGSE but not the amino acid sequence DEEEEEDDEDEEEIEVEEELCKQVRSRDISREEWKGSE. In another embodiment said antibody specifically binds to the amino acid sequence DEEEEEDDEDEEEIEVRSRDISREEWKGSE but not the amino acid sequence DEEEEEDDEDEEEIEVEEELCKQVRSRDISREEWKGSE. In another embodiment said antibody specifically binds to the amino acid sequence EEEEDDEDEEEIEVRSRDISREEWKG but not the amino acid sequence EEEEDDEDEEEIEVEEELCKQVRSRDISREEWKG. In another embodiment said antibody specifically binds to the amino acid sequence EEDDEDEEEIEVRSRDISREEW but not the amino acid sequence EEDDEDEEEIEVEEELCKQVRSRDISREEW. In another embodiment said antibody specifically binds to the amino acid sequence DDEDEEEIEVRSRDISRE but not the amino acid sequence DDEDEEEIEVEEELCKQVRSRDISRE. In another embodiment said antibody specifically binds to the amino acid sequence DEDEEEIEVRSRDISR but not the amino acid sequence DEDEEEIEVEEELCKQVRSRDISR. In another embodiment said antibody specifically binds to the amino acid sequence EDEEEIEVRSRDIS but not the amino acid sequence EDEEEIEVEEELCKQVRSRDIS. In another embodiment said antibody specifically binds to the amino acid sequence DEEEIEVRSRDI but not the amino acid sequence DEEEIEVEEELCKQVRSRDI. In another embodiment said antibody specifically binds to the amino acid sequence EIEVRSR but not the amino acid sequence EIEVEEELCKQVRSR.

In another aspect the invention provides for an isolated antibody or antigen binding fragment thereof that specifically binds to a Ciz1 b-variant polypeptide. In one embodiment said antibody is a monoclonal antibody. In another embodiment said antibody is a polyclonal antibody. In another embodiment said antibody specifically binds to a Ciz1 b-variant polypeptide but does not bind specifically bind a Ciz1 polypeptide translated from a mRNA comprising exon 14a. In another embodiment said antibody said antibody specifically binds to a Ciz1 b-variant polypeptide with an affinity at least 10 fold greater than to a Ciz1 polypeptide translated from a mRNA comprising exon 14a. In one embodiment said antibody binds with at least 100 greater affinity to a Ciz1 b-variant polypeptide as compared to a Ciz1 polypeptide translated from a mRNA comprising exon 14a. In one embodiment said antibody binds with at least 1000 greater affinity to a Ciz1 b-variant polypeptide as compared to a Ciz1 polypeptide translated from a mRNA comprising exon 14a. In one embodiment said antibody specifically binds to a contiguous epitope that includes amino acid residues encoded by exon 14b and exon 15. In one embodiment said antibody specifically binds to the amino acid sequence EGDEEEEEDDEDEEEIEVRSRDISREEWKGSETY but not the amino acid sequence EGDEEEEEDDEDEEEIEVEEELCKQVRSRDISREEWKGSETY. In another embodiment said antibody specifically binds to the amino acid sequence DEEEEEDDEDEEEIEVRSRDISREEWKGSE but not the amino acid sequence DEEEEEDDEDEEEIEVEEELCKQVRSRDISREEWKGSE. In another embodiment said antibody specifically binds to the amino acid sequence DEEEEEDDEDEEEIEVRSRDISREEWKGSE but not the amino acid sequence DEEEEEDDEDEEEIEVEEELCKQVRSRDISREEWKGSE. In another embodiment said antibody specifically binds to the amino acid sequence EEEEDDEDEEEIEVRSRDISREEWKG but not the amino acid sequence EEEEDDEDEEEIEVEEELCKQVRSRDISREEWKG. In another embodiment said antibody specifically binds to the amino acid sequence EEDDEDEEEIEVRSRDISREEW but not the amino acid sequence EEDDEDEEEIEVEEELCKQVRSRDISREEW. In another embodiment said antibody specifically binds to the amino acid sequence DDEDEEEIEVRSRDISRE but not the amino acid sequence DDEDEEEIEVEEELCKQVRSRDISRE. In another embodiment said antibody specifically binds to the amino acid sequence DEDEEEIEVRSRDISR but not the amino acid sequence DEDEEEIEVEEELCKQVRSRDISR. In another embodiment said antibody specifically binds to the amino acid sequence EDEEEIEVRSRDIS but not the amino acid sequence EDEEEIEVEEELCKQVRSRDIS. In another embodiment said antibody specifically binds to the amino acid sequence DEEEIEVRSRDI but not the amino acid sequence DEEEIEVEEELCKQVRSRDI. In another embodiment said antibody specifically binds to the amino acid sequence EIEVRSR but not the amino acid sequence EIEVEEELCKQVRSR.

In another aspect of the invention there is provided a hybridoma cell line that produces a monoclonal antibody or antigen binding fragment thereof according to the invention.

Another aspect of the present invention is a method of predicting or determining whether a lung nodule, as observed by chest X-ray, computerized tomography (CT) scan (including low dose helical (spiral) CT scan), magnetic resonance imaging (MRI), positron emission tomography (PET) scan or other imaging method, is cancerous. Lung nodules, small masses of tissue in the lung, are quite common. Although most lung nodules are noncancerous (benign), some represent early-stage lung cancer. Lung nodules usually appear as round, white shadows on a chest X-ray or CT scan. Lung nodules are usually about ⅕ inch to 1 inch, or 5 millimeters (mm) to 25 mm, in size.

In one aspect the present invention provides for a method of predicting or determining whether a lung nodule is cancerous by detecting the presence of a Ciz1 b-variant polypeptide, said method comprising the steps of:
i) providing an isolated biological sample to be tested from a human with a lung nodule;
ii) contacting said biological sample with a Ciz1 b-variant polypeptide binding agent, such as an antibody or antigen binding fragment thereof, that specifically binds said Ciz1 b-variant polypeptide;
iii) detecting the presence of said a Ciz1 b-variant polypeptide binding agent (antibody or antigen binding fragment) bound to said Ciz1 b-variant polypeptide, wherein the presence of said Ciz1 b-variant polypeptide is indicative of the presence of lung cancer.
   In one embodiment said biological sample is plasma.

Another aspect of the present invention is a method for the early detection of lung cancer in a subject, said method comprising the steps of:
i) providing an isolated biological sample to be tested;
ii) detecting the presence of a Ciz1 b-variant polypeptide,
iii) wherein the presence of said Ciz1 b-variant polypeptide indicates the presence of cancer.
   In one embodiment the lung cancer is stage 0, IA or IB NSCLC. NSCLC may be stage 0 to stage IV. Stage 0 is defined as carcinoma in situ. In stage IA, cancer is in the lung only and is 3 cm or smaller. In stage IB, the cancer is: (a) larger than 3 cm but not larger than 5 cm, (b) has spread to the main bronchus, and/or (c) has spread to the innermost layer of the lung lining. There are two stages for SCLC, limited stage and extensive stage disease. Limited stage SCLC subjects have tumors confined to the hemithorax of origin, the mediastinum, or the supraclavicular lymph nodes. is well known in the art and defined by Physician Data Query (PDQ) published by the National Cancer Institute (NCl) (Bethesda, Md., USA), incorporated by reference in its entirety.

Often it is difficult to differentially diagnosis between pneumonia and a cancerous lesion using radiology alone. Another aspect of the present invention provides for a means of differentially diagnosing whether a patient is suffering from pneumonia or lung cancer by detecting the presence of a Ciz1 b-variant polypeptide of the invention, said method comprising the steps of:
i) providing an isolated biological sample to be tested;
ii) contacting said biological sample with an antibody or antigen binding fragment thereof that specifically binds said Ciz1 b-variant polypeptide;
iii) detecting the presence of said antibody or antigen binding fragment bound to said Ciz1 b-variant polypeptide, wherein the presence of said Ciz1 b-variant polypeptide is indicative of the presence of cancer.
   In one embodiment said biological sample is selected from: a solid tissue sample, blood, plasma, serum, sputum, urine or bronchoalveolar lavage. In a further embodiment the biological sample is a solid tissue sample. In another further embodiment the biological sample is blood. In another further embodiment the biological sample is plasma. In another further embodiment the biological sample is serum. In another further embodiment the biological sample is sputum. In another further embodiment the sample is urine. In another further embodiment the biological sample is bronchoalveolar lavage. In another further embodiment the biological sample is circulating tumor cells (CTCs). In one embodiment the cancer is lung cancer. In a further embodiment the lung cancer is NSCLC. In another further embodiment the lung cancer is SCLC.

The methods for detecting cancer disclosed herein have a sensitivity at 1 standard deviation (SD) of at least 70%, at least 75%, at least 80%, at least 85%, at least 90% or at least 94%. The methods for detecting cancer disclosed herein have a specificity at 1 standard deviation of at least 70%, at least 75%, at least 80%, at least 85% or at least 90%. Sensitivity is defined as: (number of subjects correctly diagnosed as having cancer)/(total number of subjects with cancer)×100 (at 1 SD). Specificity is defined as: (number of subjects correctly diagnosed as having and not having cancer)/(total number of subjects)×100 (at 1 SD).

ROC (receiver operating characteristic) curve is plotted using sensitivity against one minus specificity at all possible intervals, to generate an area under the ROC curve (AUC). A web based calculator, e.g., available at http://www.jrocfit.org (format 5 for continuously distributed data) may be used for convenience.

Most cancer therapies (whether radiation, small molecular or biologic) are cytotoxic, either killing cells by triggering apoptosis, through necrosis or a combination of the two. Moreover, these therapies are normally not entirely specific to cancer cells, killing normal cells to a greater or lesser extent depending on the particular therapy and patient. Non-specific killing of normal cells leads to dose dependent side-effects. The extent to which normal cells are killed varies among patients, making it difficult to predict the dose at which a patient will experience a dose limiting toxicity. The ability to determine or predict when a patient has or will reach a limiting therapeutic dose would lead to better patient care. The degree of non-specific cytotoxicity or dose dependent cytotoxicity can be determined indirectly by comparing the amount of a biomarker released by a cancer cell when it dies to the amount of a biomarker that is released when either a cancer cell or normal cell dies. In one aspect the invention provides for a method of measuring non-specific cytotoxicity as a result of a cancer therapy administered to treat a cancer that expresses a Ciz1 b-variant polypeptide, by comparing the amount of a Ciz1 b-variant polypeptide, which is released from a tumor cell when it dies, to the amount of a cell death biomarker that is released from both a tumor cell or normal cell when it dies. The lower the ratio of the Ciz1 b-variant polypeptide to the cell death biomarker, the greater the non-specific cytotoxicity. In one aspect the method comprises the steps of:
i) providing an isolated biological sample to be tested;
ii) measuring the amount of said Ciz1 b-variant polypeptide present in said biological sample, wherein the presence of said Ciz1 b-variant polypeptide is indicative of cancer cell cytotoxicity;
iii) measuring the amount of a cell death biomarker, wherein the cell death biomarker is indicative of both cancer cell and normal cell cytotoxicity;
iv) comparing the amount of said Ciz1 b-variant polypeptide to the amount of said cell death biomarker.
   In one embodiment said biological sample is selected from: a solid tissue sample, blood, plasma, serum, sputum, urine or bronchoalveolar lavage. In a further embodiment the biological sample is a solid tissue sample. In another further embodiment the biological sample is blood. In another further embodiment the biological sample is plasma. In another further embodiment the biological sample is serum. In another further embodiment the biological sample is sputum. In another further embodiment the sample is urine. In another further embodiment the biological sample is bronchoalveolar lavage.

In one embodiment the cell death biomarker is a biomarker for apoptosis. In another embodiment the cell death biomarker is a biomarker for necrosis. In another embodiment the cell death biomarker is a biomarker for both apoptosis and necrosis. In one embodiment the cell death biomarker is cytokeratin 18 (CK18). In a further embodiment the method measures the amount of full length CK18. In another embodiment the method measures the amount of caspase-cleaved CK18. Antibodies and kits for measuring both full length CK18 and caspase-cleaved CK18 are commercially available. For example, M30 APOPTOSENSE, for detecting caspase-cleaved CK18, and M65 ELISA, for detecting full length CK18, are commercially available from Peviva AB (Bromma, Sweden). In another embodiment the cell death biomarker is nucleosomal DNA (nDNA) (also referred to as histone-associated DNA). Antibodies and kits for measuring nDNA are commercially available, e.g., Cell Death Detection ELISA is commercially available from Roche Diagnostics. In another embodiment the cell death marker is Cyclophilin A.

Another aspect of the present invention is a method of determining the efficacy of a cancer therapy in a subject by measuring the relative amount of said Ciz1 b-variant transcript or polypeptide before, and either or both, during and after a course of treatment. As used herein a ‘course of treatment’ refers to a prescribed regimen to be followed for a specific period of time. In one embodiment said method comprises the steps of:
i) providing a first isolated biological sample to be tested from said subject before treatment with said cancer therapy;
ii) providing a second isolated biological sample to be tested from said subject during a course of treatment with said cancer therapy;
iii) separately contacting each said biological sample with an antibody or antigen binding fragment thereof that specifically binds said Ciz1 b-variant polypeptide;
iv) measuring the amount of said Ciz1 b-variant polypeptide present in each said biological sample; wherein an increase in the amount Ciz1 b-variant polypeptide in the second sample compared to the first sample is indicative of efficacy of said cancer therapy.
   In another embodiment, said method comprises the steps of:
i) providing a first isolated biological sample to be tested from said subject before treatment with said cancer therapy;
ii) providing a second isolated biological sample to be tested from said subject after a course of treatment with said cancer therapy;
iii) separately contacting each said biological sample with an antibody or antigen binding fragment thereof that specifically binds said Ciz1 b-variant polypeptide;
iv) measuring the amount of said Ciz1 b-variant polypeptide present in each said biological sample; wherein a decrease in the amount Ciz1 b-variant polypeptide in the second sample compared to the first sample is indicative of efficacy of said cancer therapy.

In other embodiments, the above methods are modified to detect a Ciz1 b-variant transcript rather than a Ciz1 b-variant polypeptide.

According to a further aspect of the invention there is provided a kit comprising oligonucleotide primers and probes for detecting a mRNA molecule comprising a nucleic acid sequence 5′ GAAGAAGAGAUCGAGGUGAGGUCCAGAGA 3′.

In one embodiment of the invention said kit comprises oligonucleotide primers and probes comprising or consisting of the nucleic acid sequences:

5′ GAAGAGATCGAGGTGAGGTC 3′
and
5′ TGGACCTCACCTCGATCTCTTCTTCA 3′.

In a preferred embodiment of the invention said kit further comprises a thermostable DNA polymerase and deoxynucleotide triphosphates. In another embodiment said kit comprises instructions required to selectively amplify said nucleic acid molecule.

According to a further aspect of the invention there is provided a method to diagnose cancer in a subject by comparing expression of mRNA comprising a nucleotide sequence encoding a Ciz 1 replication domain to mRNA comprising a nucleotide sequence encoding a Ciz 1 immobilisation domain, said method comprising the steps:
i) providing an isolated biological sample to be tested;
ii) detecting the presence of mRNA comprising a nucleotide sequence encoding Ciz 1 replication domain;
iii) detecting the presence of mRNA comprising a nucleotide sequence encoding Ciz 1 immobilisation domain;
iv) comparing the relative expression of said mRNA comprising a nucleotide sequence encoding said Ciz 1 replication domain to said mRNA comprising a nucleotide sequence encoding said Ciz 1 immobilisation domain; wherein a difference in relative expression of at least 2 fold is indicative of cancer.

In one embodiment of the invention there is provided a method to diagnose cancer in a subject by comparing expression of mRNA comprising a nucleotide sequence of SEQ ID NO: 12 to mRNA comprising a nucleotide sequence SEQ ID NO: 18, said method comprising the steps:
i) providing an isolated biological sample to be tested;
ii) detecting the presence of mRNA comprising a nucleotide sequence of SEQ ID NO: 12;
iii) detecting the presence of mRNA comprising a nucleotide sequence of SEQ ID NO: 18;
iv) comparing the relative expression of said mRNA comprising a nucleotide sequence of SEQ ID NO: 12 to said mRNA comprising a nucleotide sequence of SEQ ID NO: 18; wherein a difference in relative expression of at least 2 fold is indicative of cancer.

In another embodiment of the invention there is provided a method to diagnose cancer in a subject by comparing expression of mRNA comprising a nucleotide sequence of SEQ ID NO: 13 to mRNA comprising a nucleotide sequence SEQ ID NO: 19, said method comprising the steps:
i) providing an isolated biological sample to be tested;
ii) detecting the presence of mRNA comprising a nucleotide sequence of SEQ ID NO: 13;
iii) detecting the presence of mRNA comprising a nucleotide sequence of SEQ ID NO: 19;
iv) comparing the relative expression of said mRNA comprising a nucleotide sequence of SEQ ID NO: 13 to said mRNA comprising a nucleotide sequence of SEQ ID NO: 19; wherein a difference in relative expression of at least 2 fold is indicative of cancer.

In another embodiment of the invention there is provided a method to diagnose cancer in a subject by comparing expression of mRNA comprising a nucleotide sequence of SEQ ID NO: 14 to mRNA comprising a nucleotide sequence SEQ ID NO: 20, said method comprising the steps:
i) providing an isolated biological sample to be tested;
ii) detecting the presence of mRNA comprising a nucleotide sequence of SEQ ID NO: 14;
iii) detecting the presence of mRNA comprising a nucleotide sequence of SEQ ID NO: 20;
iv) comparing the relative expression of said mRNA comprising a nucleotide sequence of SEQ ID NO: 14 to said mRNA comprising a nucleotide sequence of SEQ ID NO: 20; wherein a difference in relative expression of at least 2 fold is indicative of cancer.

In one embodiment, the method uses polymerase chain reaction (PCR) to detect the presence of said Ciz1 replication and immobilisation domains. In another embodiment the method further comprises the steps of: forming a preparation comprising said sample and an oligonucleotide primer pair suitable to amplify all or a portion of said Ciz1 replication and an oligonucleotide primer pair suitable to amplify all or a portion of said Ciz1 immobilisation domain, and performing a polymerase chain reaction on said sample.

In one embodiment the cancer is a lung, breast, kidney, thyroid, melanoma, liver, bladder or colon cancer. In one embodiment the cancer is non-small cell lung cancer (NSCLC). In another embodiment the cancer is breast cancer. In another embodiment the cancer is kidney cancer. In another embodiment the cancer is colon cancer.

In one embodiment said oligonucleotide primer pair that amplifies the Ciz 1 replication domain is selected from the group consisting of:

CACAACTGGCCACTCCAAAT with CCTCTACCACCCCCAATCG;
and
ACACACCAGAAGACCAAGATTTACC with TGCTGGAGTGCGTTTTTC
CT.

In another embodiment said amplified replication domain is detected with an oligonucleotide comprising the sequence:

CGCCAGTCCTTGCTGGGACC
or
CCCTGCCCAGAGGACATCGCC

In another embodiment said oligonucleotide primer pair that amplifies the Ciz 1 immobilization domain is selected from the group consisting of:

CAGGGGCATAAGGACAAAG with GGCTTCCTCAGACCCCTCTG;
and
CGAGGGTGATGAAGAAGAGGA with CCCCTGAGTTGCTGTGATA.

In another embodiment said amplified immobilization domain is detected with an oligonucleotide comprising the sequence:

TGGTCCTCATCTTGGCCAGCA,
CACGGGCACCAGGAAGTCCA
or
CACTGCAAGTCCCTGGGCCA.

In another said method is combined with an analysis of expression of a Ciz 1 b-variant transcript according to the invention.

In a preferred method of the invention said method of diagnosis is combined with a method of treatment according to the invention.

In one aspect of the invention there is provided a method to diagnose cancer in a subject by comparing the expression of a polypeptide comprising a Ciz 1 replication domain and a polypeptide comprising a Ciz 1 immobilisation domain, said method comprising the steps of:
i) providing an isolated biological sample to be tested;
ii) detecting the presence of said Ciz 1 replication domain and Ciz 1 immobilisation domain,
iii) comparing the relative amount of said Ciz 1 replication domain to said Ciz 1 immobilisation domain present in said sample; wherein a difference of greater than 2 fold in the relative amount of Ciz 1 replication domain to said Ciz 1 immobilisation domain is indicative of the presence of cancer.

In embodiment of the invention there is provided a method to diagnose cancer in a subject by comparing the expression of a Ciz 1 polypeptide comprising the amino acid sequence of SEQ ID NO: 9 and Ciz 1 polypeptide comprising the amino acid sequence of SEQ ID NO: 15, said method comprising the steps of:
i) providing an isolated biological sample to be tested;
ii) detecting the presence of said Ciz 1 polypeptide comprising the amino acid sequence of SEQ ID NO: 9 and Ciz 1 polypeptide comprising the amino acid sequence of SEQ ID NO: 15,
iii) comparing the relative amount of said Ciz 1 polypeptide comprising the amino acid sequence of SEQ ID NO: 9 to said Ciz 1 polypeptide comprising the amino acid sequence of SEQ ID NO: 15 present in said sample; wherein a difference of greater than 2 fold in the relative amount of said Ciz 1 polypeptide comprising the amino acid sequence of SEQ ID NO: 9 to said Ciz 1 polypeptide comprising the amino acid sequence of SEQ ID NO: 15 is indicative of the presence of cancer.

In another embodiment of the invention there is provided a method to diagnose cancer in a subject by comparing the expression of a Ciz 1 polypeptide comprising the amino acid sequence of SEQ ID NO: 10 and Ciz 1 polypeptide comprising the amino acid sequence of SEQ ID NO: 16, said method comprising the steps of:
i) providing an isolated biological sample to be tested;
ii) detecting the presence of said Ciz 1 polypeptide comprising the amino acid sequence of SEQ ID NO: 10 and Ciz 1 polypeptide comprising the amino acid sequence of SEQ ID NO: 16,
iii) comparing the relative amount of said Ciz 1 polypeptide comprising the amino acid sequence of SEQ ID NO: 10 to said Ciz 1 polypeptide comprising the amino acid sequence of SEQ ID NO: 16 present in said sample; wherein a difference of greater than 2 fold in the relative amount of said Ciz 1 polypeptide comprising the amino acid sequence of SEQ ID NO: 10 to said Ciz 1 polypeptide comprising the amino acid sequence of SEQ ID NO: 16 is indicative of the presence of cancer.

In another embodiment of the invention there is provided a method to diagnose cancer in a subject by comparing the expression of a Ciz 1 polypeptide comprising the amino acid sequence of SEQ ID NO: 11 and Ciz 1 polypeptide comprising the amino acid sequence of SEQ ID NO: 17, said method comprising the steps of:
i) providing an isolated biological sample to be tested;
ii) detecting the presence of said Ciz 1 polypeptide comprising the amino acid sequence of SEQ ID NO: 11 and Ciz 1 polypeptide comprising the amino acid sequence of SEQ ID NO: 17,
iii) comparing the relative amount of said Ciz 1 polypeptide comprising the amino acid sequence of SEQ ID NO: 11 to said Ciz 1 polypeptide comprising the amino acid sequence of SEQ ID NO: 17 present in said sample; wherein a difference of greater than 2 fold in the relative amount of said Ciz 1 polypeptide comprising the amino acid sequence of SEQ ID NO: 11 to said Ciz 1 polypeptide comprising the amino acid sequence of SEQ ID NO: 17 is indicative of the presence of cancer.

In another embodiment of the invention there is provided a method to diagnose cancer in a subject by comparing the expression of a polypeptide comprising a Ciz 1 replication domain and a polypeptide comprising a Ciz 1 immobilisation domain, said method comprising the steps of:
i) providing an isolated biological sample to be tested;
ii) contacting said biological sample with an antibody or antigen binding fragment thereof that specifically binds said Ciz 1 polypeptide replication domain;
iii) contacting said biological sample with an antibody or antigen binding fragment thereof that specifically binds said Ciz 1 polypeptide immobilisation domain;
iv) detecting the presence of said antibody or antigen binding fragment bound to said Ciz 1 polypeptide replication domain and bound to said Ciz 1 polypeptide immobilisation domain,
v) comparing the relative amount of said Ciz 1 polypeptide replication domain to said Ciz 1 polypeptide immobilisation domain present in said sample; wherein a difference of greater than 2 fold in the relative amount of said Ciz 1 polypeptide replication domain to said Ciz 1 polypeptide immobilisation domain is indicative of the presence of cancer.

In one embodiment the presence of at least 2 fold more replication domain than immobilisation domain is indicative of a metastatic cancer.

According to a further aspect of the invention there is provided a kit comprising oligonucleotide primers adapted to specifically amplify a nucleic acid molecule comprising the replication domain of Ciz 1 and the immobilisation domain of Ciz 1.

In one embodiment of the invention said oligonucleotide primers that amplify the replication domain are:

CACAACTGGCCACTCCAAAT with CCTCTACCACCCCCAATCG;
or
ACACACCAGAAGACCAAGATTTACC with TGCTGGAGTGCGTTTTTC
CT.

In a preferred method of the invention said oligonucleotide primers that amplify the immobilization domain are:

CAGGGGCATAAGGACAAAG with GGCTTCCTCAGACCCCTCTG;
or
CGAGGGTGATGAAGAAGAGGA with CCCCTGAGTTGCTGTGATA.

In a preferred embodiment of the invention said kit includes oligonucleotide probes that detect the amplified Ciz 1 replication domain and are selected from:

CGCCAGTCCTTGCTGGGACC
or
CCCTGCCCAGAGGACATCGCC

In a preferred embodiment of the invention said kit includes oligonucleotide probes that detect the amplified Ciz 1 immobilization domain and are selected from:

TGGTCCTCATCTTGGCCAGCA,
CACGGGCACCAGGAAGTCCA
or
CACTGCAAGTCCCTGGGCCA.

According to a further aspect of the invention there is provided a kit comprising a first antibody or antigen binding fragment thereof that specifically binds the replication domain of Ciz 1 protein and a second antibody or antigen binding fragment thereof that specifically binds the immobilization domain of Ciz 1 protein.

Another aspect of the invention relates to use of the above methods comprising the detection of a Ciz1 replication domain and immobilization domain (or mRNAs encoding the same) for indicating the prognosis of a cancer patient. In some embodiments, the above methods measure the relative levels in tissue adjacent to a tumor rather than the tumor itself, wherein patients with at least 2 fold more replication domain than immobilisation domain have a poorer prognosis compared with patients with less than a 2 fold difference. In some embodiments, the adjacent tissue is within 20 mm, 15 mm, 10 mm or 5 mm of the tumor margin.

In a preferred embodiment of the invention said antibody is a monoclonal antibody.

An antibody that binds to a Ciz1 polypeptide of the present invention is preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” which refers to an antibody that is produced as a single molecular species, e.g., from a population of homogenous isolated cells. A “monoclonal antibody composition” refers to a preparation of antibodies or fragments thereof of in a composition that includes a single molecular species of antibody. In one embodiment, a monoclonal antibody is produced by a mammalian cell. One or more monoclonal antibody species may be combined. An antibody of the present invention may be recombinant or produced using hybridoma technology.

The Ciz1 polypeptide binding antibodies can be full-length (e.g., an IgG (e.g., an IgG1, IgG2, IgG3, IgG4), IgM, IgA (e.g., IgA1, IgA2), IgD, and IgE) or can include only an antigen-binding fragment (e.g., a Fab, Fab′, F(ab′)₂ or scFv fragment), e.g., it does not include an Fc domain or a CH2, CH3, or CH4 sequence. The antibody can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. A Ciz1 polypeptide of the present invention-binding antibody can include a heavy and light chain constant region substantially from a human antibody, e.g., a human IgG1 constant region or a portion thereof, or from another species, including but not limited to, mouse, rat, dog, cat, goat, sheep, cow, horse, chicken or guinea pig.

In one embodiment, the antibody (or fragment thereof) is a recombinant or modified antibody, e.g., a chimeric, a humanized, a deimmunized, or an in vitro generated antibody. The term “recombinant” or “modified” antibody, as used herein, is intended to include all antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of immunoglobulin gene sequences to other DNA sequences. Such recombinant antibodies include humanized, CDR grafted, chimeric, deimmunized, in vitro generated antibodies, and may optionally include constant regions derived from human germline immunoglobulin sequences.

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. In another example the antibody is a camel single domain VH antibody. The term “antibody” encompasses antigen-binding fragments of antibodies (e.g., single chain antibodies, Fab fragments, F(ab′)₂, a Fd fragment, a Fv fragments, and dAb fragments) as well as complete antibodies.

The VH and VL regions can be further subdivided into regions of hypervariability, termed “complementarity determining regions” (CDR), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two beta-sheets formed of about seven beta-strands, and a conserved disulphide bond (see, e.g., A. F. Williams and A. N. Barclay 1988 Ann. Rev Immunol. 6:381-405). The canonical structures of hypervariable loops of an immunoglobulin variable can be inferred from its sequence, as described in Chothia et al. (1992) J. Mol. Biol. 227:799-817; Tomlinson et al. (1992) J. Mol. Biol. 227:776-798); and Tomlinson et al. (1995) EMBO J. 14(18):4628-38.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form a target binding structure (or “antigen binding site”), e.g., a structure that interacts with a Ciz1 polypeptide of the present invention, e.g., binds to or inhibits a Ciz1 polypeptide of the present invention (e.g., b-variant).

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region includes three domains, CH1, CH2 and CH3. The light chain constant region includes a CL domain. The variable region of the heavy and light chains contains a binding domain that interacts with an antigen. The constant regions of the antibodies typically mediate the binding of the antibody to a host tissue or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof). In one embodiment the antibody is an IgA. In another embodiment the antigody is an IgG. In another embodiment the antigody is an IgE. In another embodiment the antigody is an IgD. In another embodiment the antigody is an IgM. The light chains of the immunoglobulin may be of types kappa or lambda. In one embodiment, the antibody is glycosylated. An antibody can be functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human or effectively human. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human or effectively human. For example, FR1, FR2, and FR3 collectively can be at least 70, 75, 80, 85, 90, 92, 95, 98, or 99% identical to a human sequence encoded by a human germline V segment of a locus encoding a light or heavy chain sequence.

All or part of an antibody can be encoded by an immunoglobulin gene or a segment thereof. Exemplary immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin light chains (about 25 Kd or 214 amino acids) are encoded by a variable region gene at the NH2-terminus (about 110 amino acids) and a kappa or lambda constant region gene at the COOH-terminus. Full-length immunoglobulin heavy chains (about 50 Kd or 446 amino acids), are similarly encoded by a variable region gene (about 116 amino acids) and one of the other aforementioned constant region genes, e.g., gamma (encoding about 330 amino acids). A light chain refers to any polypeptide that includes a light chain variable domain. A heavy chain refers to any polypeptide that a heavy chain variable domain.

The term “antigen-binding fragment” of a full-length antibody (or simply “antibody portion,” or “fragment”), as used herein, refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883.

A “humanized” immunoglobulin variable region is an immunoglobulin variable region that includes sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

As used herein, “binding affinity” refers to the apparent association constant or Ka. Biding affinity may be expressed as the dissociation constant (Kd) which is the reciprocal of the Ka. A target binding agent, such as an antibody may, for example, have a Kd of less than 10⁻⁵, 10⁻⁶, 10⁻⁷ or 10⁻⁸ M for a particular target molecule. Differences in binding affinity (e.g., for specificity or other comparisons) can be, e.g., at least 1.5, 2, 5, 10, 50, 100, or 1000-fold. For example, a Ciz1 polypeptide-binding protein may preferentially bind to Ciz1 b-variant at least 1.5, 2, 5, 10, 50, 100, or 1000-fold better than to another a Ciz1 polypeptide comprising a amino acid sequence encoded by exon 14a instead of 14b. A Ciz1 polypeptide-binding protein may also be species-specific or species-general (e.g., can bind to a Ciz1 polypeptide of the present invention from more than one species or can be specific for a human Ciz1 polypeptide such as human Ciz1 b-variant).

Binding affinity can be determined by a variety of methods including equilibrium dialysis, equilibrium binding, gel filtration, ELISA, surface plasmon resonance, or spectroscopy (e.g., using a fluorescence assay). These techniques can be used to measure the concentration of bound and free ligand as a function of ligand (or target) concentration. The concentration of bound ligand ([Bound]) is related to the concentration of free ligand ([Free]) and the concentration of binding sites for the ligand on the target where (N) is the number of binding sites per target molecule by the following equation:

[Bound]=N[Free]/((1/Ka)+[Free])

Although quantitative measurements of Ka are routine, it is not always necessary to make an exact determination of Ka, though, since sometimes it is sufficient to obtain a qualitative measurement of affinity, e.g., determined using a method such as ELISA or FACS analysis, is proportional to Ka, and thus can be used for comparisons, such as determining whether a higher affinity is, e.g., 2, 5, 10, 20, or 50 fold higher than a reference. Binding affinity is typically evaluated in 0.01 M HEPES pH 7.4, 0.15 M NaCl, 3 mM EDTA and 0.005% (v/v) surfactant P20.

Protein Production. Standard recombinant nucleic acid methods can be used to express an antibody or antigen binding fragment that binds to Ciz1 polypeptide of the present invention. See, for example, the techniques described in Sambrook & Russell, Molecular Cloning: A Laboratory Manual, 3rd Edition, Cold Spring Harbor Laboratory, N.Y. (2001) and Ausubel et al., Current Protocols in Molecular Biology (Greene Publishing Associates and Wiley Interscience, N.Y. (1989). Generally, a nucleic acid sequence encoding the binding proteins cloned into a nucleic acid expression vector. If the protein includes multiple polypeptide chains, each chain can be cloned into an expression vector, e.g., the same or different vectors, that are expressed in the same or different cells. Methods for producing antibodies are also provided below. Some antibodies, e.g., Fabs, can be produced in bacterial cells, e.g., E. coli cells. Antibodies can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one embodiment, antibodies are produced in mammalian cells. Preferred mammalian host cells for expressing the clone antibodies or antigen-binding fragments thereof include Chinese Hamster Ovary (CHO cells) (including dhfr- CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NSO myeloma cells, SP2 cells, COS cells, HEK 293T cells, and a cell from a transgenic animal, e.g., a transgenic mammal. For example, the cell is a mammary epithelial cell.

In addition to the nucleic acid sequence encoding the immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pats. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr- host cells with methotrexate selection/amplification) and the neo gene (for G418 selection). Another exemplary expression system is the glutamine synthase (GS) vector system available from Lonza Group Ltd. CH (see, e.g., Clark et al. (2004) BioProcess International 2(4):48-52; Barnes et al. (2002) Biotech Bioeng. 81(6):631-639).

In an exemplary system for recombinant expression of an antibody, or antigen-binding portion thereof, a recombinant expression vector encoding both the antibody heavy chain and the antibody light chain is introduced into dhfr- CHO cells, e.g., by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G.

The codon usage can be adapted to the codon bias of the host cell, e.g., for CHO cells it can be adapted for the codon bias Cricetulus griseus genes. In addition, regions of very high (>80%) or very low (<30%) GC content can be avoid avoided where possible. During the optimization process following cis-acting sequence motifs were avoided: internal TATA-boxes; chi-sites and ribosomal entry sites; AT-rich or GC-rich sequence stretches; ARE, INS, CRS sequence elements; repeat sequences and RNA secondary structures; and (cryptic) splice donor and acceptor sites, branch points. Two STOP codons can be used to ensure efficient termination. The codon optimization of the sequence can be evaluated according to Sharp, P. M., Li, W. H., Nucleic Acids Res. 15 (3), 1987). The standard codon adaptation index (CAI) can be used. Rare codons include those with a quality class between 0-40.

Aptamers. In one embodiment, the invention also features target protein-binding agents such as aptamers. Aptamers may be nucleic acid aptamers or peptide aptamers. The term “nucleic acid aptamer,” as used herein, refers to a nucleic acid molecule which has a conformation that includes an internal non-duplex nucleic acid structure of at least 5 nucleotides. An aptamer can be a single-stranded nucleic acid molecule which has regions of self-complementarity. “Peptide aptamers” are short peptide sequences presented and conformationally constrained in a robust, inert protein scaffold (Evans et al., Journal of Biology 2008, 7:3, incorporated in its entirety). The three-dimensional conformational constraint of the inserted peptide applied by the protein scaffold reatly increases the affinity of the aptamer for the target over that of an unconstrained peptide sequence. Exemplary aptamers include nucleic acid molecules and peptides that bind to a Ciz1 polypeptide of the present invention (e.g., b-variant). Particular aptamers may be used in place of an antibody in many cases. Other peptides that bind a Ciz1 polypeptide of the invention are also included. Peptide like molecules such as peptoids are further included in the invention. “Peptoids”, or poly-N-substituted glycines, are a class of peptidomimetics whose side chains are appended to the nitrogen atom of the peptide backbone, rather than to the α-carbons (as they are in amino acids). T-cell receptors can also be used as target binding agents.

The term “binding agent” refers to an agent capable of binding to a Ciz1 polypeptide (e.g., Ciz1 b-variant) of the present invention under experimental conditions and include, but are not limited to, antibodies and antigen antibody binding fragments thereof, including but not limited to Fab, Fab′, F(ab′)₂, scFv or single-domain antibody (sdAb), (also referred to as a nanobody), nucleic acid aptamers, and peptide aptamers. The Ciz1 polypeptide binding agents have in vitro and in vivo diagnostic utilities. For example, measurement of levels of a Ciz1 polypeptide in samples derived from a subject can be used for the diagnosis of diseases such as cancer. Moreover, the monitoring and quantitation of a Ciz1 polypeptide level can be used prognostically to stage the progression of the disease and to evaluate the efficacy of agents used to treat a cancer subject.

In one aspect, a biological sample which may contain a Ciz1 polypeptide, such as lung tissue or other biological tissue, is obtained from a subject suspected of having a particular cancer or risk for cancer. Aliquots of whole tissues, or cells, are solubilized using any one of a variety of solubilization cocktails known to those skilled in the art. For example, tissue can be solubilized by addition of lysis buffer comprising (per liter) 8 M urea, 20 ml of Nonidet P-40 surfactant, 20 ml of ampholytes (pH 3.5-10), 20 ml of 2-mecaptoethanol, and 0.2 mM of phenylmethylsulfonyl fluoride (PMSF) in distilled deionized water.

In one aspect, the invention provides a diagnostic method for detecting the presence of a Ciz1 polypeptide of the present invention, in vitro (e.g., a biological sample, such as tissue, biopsy, e.g., a cancerous tissue) or in vivo (e.g., in vivo imaging in a subject). The method includes: (i) contacting a sample with a Ciz1 polypeptide of the present invention-binding agent (e.g., antibody, antigen-binding fragment or aptamer); and (ii) detecting formation of a complex between the Ciz1 polypeptide-binding agent and the sample. The method can also include contacting a reference sample (e.g., a control sample) with the binding agent, and determining the extent of formation of the complex between the binding agent and the sample relative to the same for the reference sample. A change, e.g., a statistically significant change, in the formation of the complex in the sample or subject relative to the control sample or subject can be indicative of the presence of a Ciz1 polypeptide of the present invention (e.g., b-variant) in the sample. The Ciz1 polypeptide of the present invention-binding agent can be directly or indirectly labeled with a detectable substance to facilitate detection of the bound or unbound antibody. Suitable detectable substances include various enzymes, prosthetic groups, fluorescent materials, luminescent materials and radioactive materials.

In some embodiments of the aspects described herein, an agent specific for a Ciz1 polypeptide, such as an antibody or antigen-binding fragment thereof, a natural or recombinant ligand, a small molecule, or a modifying moiety, is directly labeled with a tag to facilitate the detection of the modification. The terms “label” or “tag”, as used herein, refer to a composition capable of producing a detectable signal indicative of the presence of a target, such as, the presence of a specific modification in a biological sample. Suitable labels include fluorescent molecules, radioisotopes, nucleotide chromophores, enzymes, substrates, chemiluminescent moieties, magnetic particles, bioluminescent moieties, peptide tags (c-Myc, HA, VSV-G, HSV, FLAG, V5 or HIS) and the like. As such, a label is any composition detectable by spectroscopic, photochemical, biochemical, immunochemical, electrical, optical or chemical means needed for the methods to identify the Ciz1 polypeptide. In some embodiments of the aspects described herein, the modification moiety itself may be labeled directly. For example, one can use a radioactive label or a florescent label so that the protein modification can be read directly (or in combination with other modifications) without the use of antibodies. Naturally, also antibodies may be labeled to assist in their direct detection.

The terms “labeled antibody” or “tagged antibody”, as used herein, includes antibodies that are labeled by detectable means and include, but are not limited to, antibodies that are fluorescently, enzymatically, radioactively, and chemiluminescently labeled. Antibodies can also be labeled with a detectable tag, such as c-Myc, HA, VSV-G, HSV, FLAG, V5, or HIS, which can be detected using an antibody specific to the tag, for example, an anti-c-Myc antibody. Antibodies can also be labeled with an enzyme (e.g., alkaline phosphatase, acid phosphatase, horseradish peroxidase, beta-galactosidase and ribonuclease). Various methods of labeling binding agents are known in the art and may be used. Non-limiting examples of fluorescent labels or tags for labeling the antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyl]protein, TruRed (PerCP-Cy5.5 conjugate), Fluor X, Fluoresceinisothyocyanate

(FITC), BOD1PY-FL, TR1TC, X-Rhodamine (XR1TC), L is samine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cyt, Cy3, Cy3B, Cy3.5, Cy5, Cy5.5 or Cy7. A variety of suitable fluorescers and chromophores are described by Stryer (1968) Science, 162:526 and Brand, L. et al. (1972) Annual Review of Biochemistry, 41:843-868. The binding proteins can be labeled with fluorescent chromophore groups by conventional procedures such as those disclosed in U.S. Pat. Nos. 3,940,475, 4,289,747, and 4,376,110. In one embodiment the fluorescers is a xanthene dye, which include the fluoresceins and rhodamines. In another embodiment the fluorescent compounds are the naphthylamines. Once labeled with a fluorophore or chromophore, the binding protein can be used to detect the presence or localization of the Ciz1 polypeptide of the present invention in a sample, e.g., using fluorescent microscopy. In one embodiment the fluorescent microscopy is confocal or deconvolution microscopy. Likewise, a bioluminescent compound may be used to label the Ciz1 antibody. The presence of a bioluminescence protein is determined by detecting the presence of luminescence. Important bioluminescence compounds for purposes of labeling are luciferin, luciferase and aequorin.

In a specific embodiment of the invention, the levels of a Ciz1 polypeptide in biological samples can be analyzed by two-dimensional gel electrophoresis. Methods of two-dimensional electrophoresis are known to those skilled in the art. Biological samples, such as tissue samples, are loaded onto electrophoretic gels for isoelectric focusing separation in the first dimension which separates proteins based on charge. A number of first-dimension gel preparations may be utilized including tube gels for carrier ampholytes-based separations or gels strips for immobilized gradients based separations. After first-dimension separation, proteins are transferred onto the second dimension gel, following an equilibration procedure and separated using SDS PAGE which separates the proteins based on molecular weight. When comparing biological samples derived from different subjects, multiple gels are prepared from individual biological samples (including samples from normal controls).

Following separation, the proteins are transferred from the two-dimensional gels onto membranes commonly used for Western blotting. The techniques of Western blotting and subsequent visualization of proteins are also well known in the art (Sambrook et al, “Molecular Cloning, A Laboratory Manual”, 2.sup.nd Edition, Volume 3, 1989, Cold Spring Harbor). The standard procedures may be used, or the procedures may be modified as known in the art for identification of proteins of particular types, such as highly basic or acidic, or lipid soluble, etc. (See for example, Ausubel, et al., 1999, Current Protocols in Molecular Biology, Wiley & Sons, Inc., N.Y.). Antibodies that bind to the a Ciz1 polypeptide are utilized in an incubation step, as in the procedure of Western blot analysis. A second antibody specific for the first antibody is utilized in the procedure of Western blot analysis to visualize proteins that reacted with the first antibody.

The detection of a Ciz1 polypeptide levels in biological samples can also be used to monitor the efficacy of potential anti-cancer agents during treatment. For example, the level of a Ciz1 polypeptide production can be determined before and during treatment. The efficacy of the agent can be followed by comparing Ciz1 expression throughout the treatment. Agents exhibiting efficacy are those which decrease the level of a Ciz1 polypeptide production as treatment with the agent progresses.

Complex formation between a Ciz1 polypeptide-binding agent and a Ciz1 polypeptide of the present invention (e.g., b-variant) can be detected by measuring or visualizing either the binding agent bound to the Ciz1 polypeptide or unbound binding agent. Assays, e.g., immunoassays, of the invention include competitive and non-competitive (“sandwich”) assays. Immunoassays of the invention include but are not limited to assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, flow cytometry or tissue immunohistochemistry to name but a few. Further to labeling the Ciz1 polypeptide-binding agent, the presence of a Ciz1 polypeptide of the present invention can be assayed in a sample by a competition immunoassay utilizing standards labeled with a detectable substance and an unlabeled Ciz1 polypeptide-binding agent. In one example of this assay, the biological sample, the labeled standards and the Ciz1 polypeptide-binding agent are combined and the amount of labeled standard bound to the unlabeled binding agent is determined. The amount of Ciz1 polypeptide of the present invention in the sample is inversely proportional to the amount of labeled standard bound to the Ciz1 polypeptide-binding agent.

Histological Analysis. Immunohistochemistry can be performed using a Ciz1 polypeptide-binding agent (e.g., antibody, antigen binding fragment thereof or aptamer). For example, in the case of an antibody, the antibody can be synthesized with a label (such as a purification or epitope tag), or can be detectably labeled, e.g., by conjugating a label or label-binding group. For example, a chelator can be attached to the antibody. The antibody is then contacted to a histological preparation, e.g., a fixed section of tissue that is on a microscope slide. After an incubation for binding, the preparation is washed to remove unbound antibody. The preparation is then analyzed, e.g., using microscopy, to identify if the antibody bound to the preparation. The method can be used to evaluate a cell or tissue (e.g., cancer cell or solid tumor tissue sample). The antibody (or other polypeptide or peptide) can be unlabeled at the time of binding. After binding and washing, the antibody is labelled in order to render it detectable. Protein Arrays. The Ciz1 polypeptide-binding agent can also be immobilized on an array (e.g., protein array or microarray). The array can be used as a diagnostic tool, e.g., to screen medical samples (such as isolated cells, blood, plasma, serum, urine, sputum, biopsies, and the like). Of course, the array can also include other binding proteins, e.g., that bind to Ciz1 polypeptide of the present invention or to other target molecules.

Methods of producing polypeptide arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati. The array substrate can be, for example, nitrocellulose, plastic, glass, e.g., surface-modified glass. The array can also include a porous matrix, e.g., acrylamide, agarose, or another polymer.

For example, the array can be an array of antibodies, e.g., as described in De Wildt, supra. Cells that produce the binding proteins can be grown on a filter in an arrayed format. Polypeptide production is induced, and the expressed polypeptides are immobilized to the filter at the location of the cell. A protein array can be contacted with a labeled target to determine the extent of binding of the target to each immobilized polypeptide. If the target is unlabeled, a sandwich method can be used, e.g., using a labeled probed, to detect binding of the unlabeled target. Information about the extent of binding at each address of the array can be stored as a profile, e.g., in a computer database. The protein array can be produced in replicates and used to compare binding profiles, e.g., of a target and a non-target.

FACS. (Fluorescent Activated Cell Sorting). The Ciz1 polypeptide-binding agent can be used to label cells or protein, e.g., cells or protein in a biological sample such as a patient sample. The binding protein can also be attached (or attachable) to a fluorescent compound. The cells can then be sorted using fluorescent activated cell sorted (e.g., using a sorter available from Becton Dickinson Immunocytometry Systems, San Jose Calif.; see also U.S. Pat. Nos. 5,627,037; 5,030,002; and 5,137,809). As cells pass through the sorter, a laser beam excites the fluorescent compound while a detector counts cells that pass through and determines whether a fluorescent compound is attached to the cell by detecting fluorescence. The amount of label bound to each cell can be quantified and analyzed to characterize the sample. The sorter can also deflect the cell and separate cells bound by the binding protein from those cells not bound. The separated cells can be cultured and/or characterized.

In Vivo Imaging. In still another embodiment, the invention provides a method for detecting the presence of a Ciz1 polypeptide(e.g., b-variant)-expressing cancerous tissues in vivo or remnants thereof. The method includes: administering the Ciz1 polypeptide-binding agent to a subject; and detecting the Ciz1 polypeptide-binding agent in the subject. The detecting can include determining location or time of formation of the complex. The method can include scanning or otherwise imaging the subject, e.g., a region of the subject's body. Another method includes (i) administering to a subject (e.g., a patient having a cancer or neoplastic disorder) a Ciz1 polypeptide-binding antibody, conjugated to a detectable marker; (ii) exposing the subject to a means for detecting said detectable marker to the Ciz1 polypeptide-expressing tissues or cells. For example, the method can be used visualize Ciz1 b-variant released from dead or dying cancer cells in a patients. The subject can be imaged, e.g., by NMR or other tomographic means. Examples of labels useful for diagnostic imaging include radiolabels such as 131I, 111In, 123I, 99 mTc, 32P, 125I, 3H, 14C, and 188Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The binding agent can be labeled with such reagents using known techniques. For example, see Wensel and Meares (1983) Radioimmunoimaging and Radioimmunotherapy, Elsevier, N.Y. for techniques relating to the radiolabeling of antibodies and D. Colcher et al. (1986) Meth. Enzymol. 121: 802-816.

A radiolabeled binding agent can also be used for in vitro diagnostic tests. The specific activity of an isotopically-labeled protein depends upon the half-life, the isotopic purity of the radioactive label, and how the label is incorporated into the protein.

Also included in the invention are kits including the binding agent that binds to a Ciz1 polypeptide of the present invention and instructions for diagnostic use, e.g., the use of the target-binding agent (e.g., antibody or antigen-binding fragment thereof, or other polypeptide or peptide or aptamer) to detect Ciz1 polypeptide of the present invention, in vitro, e.g., in a sample, e.g., a biopsy or cells from a patient having a cancer or neoplastic disorder, or in vivo, e.g., by imaging a subject. The kit can further contain a least one additional reagent, such as a label or additional diagnostic agent. For in vivo use the binding protein can be formulated as a pharmaceutical composition.

In one embodiment the invention provides for an isolated antibody, or antigen-binding fragment thereof, that binds to a human Ciz1 polypeptide or antigen of the present invention with an affinity K_{D} of less than 1×10⁻⁸ M. In another embodiment the invention provides for an isolated antibody, or antigen-binding fragment thereof, that binds to a human Ciz1 polypeptide or antigen of the present invention with an affinity K_{D} of less than 5×10⁻⁹ M. In another embodiment the invention provides for an isolated antibody, or antigen-binding fragment thereof, that binds to a human Ciz1 polypeptide or antigen of the present invention with an affinity K_{D} of less than 1×10⁻⁹ M. In one embodiment, isolated antibody, or antigen-binding fragment thereof is a human antibody, or antigen-binding fragment thereof. In one embodiment, isolated antibody, or antigen-binding fragment thereof is a mouse antibody, or antigen-binding fragment thereof. In one embodiment, isolated antibody, or antigen-binding fragment thereof is a rat antibody, or antigen-binding fragment thereof. In one embodiment, isolated antibody, or antigen-binding fragment thereof is a rabbit antibody, or antigen-binding fragment thereof. In one embodiment, isolated antibody, or antigen-binding fragment thereof is a guinea pig antibody, or antigen-binding fragment thereof. In one embodiment, isolated antibody, or antigen-binding fragment thereof is a goat antibody, or antigen-binding fragment thereof. In one embodiment, isolated antibody, or antigen-binding fragment thereof is a sheep antibody, or antigen-binding fragment thereof. In one embodiment, isolated antibody, or antigen-binding fragment thereof is a bovine antibody, or antigen-binding fragment thereof. In one embodiment, isolated antibody, or antigen-binding fragment thereof is an equine antibody, or antigen-binding fragment thereof. In one embodiment, isolated antibody, or antigen-binding fragment thereof is a chicken antibody, or antigen-binding fragment thereof. In one embodiment, isolated antibody, or antigen-binding fragment thereof is a porcine antibody, or antigen-binding fragment thereof. In one embodiment, isolated antibody, or antigen-binding fragment thereof is a feline antibody, or antigen-binding fragment thereof. In one embodiment, isolated antibody, or antigen-binding fragment thereof is a canine antibody, or antigen-binding fragment thereof. In one embodiment, isolated antibody, or antigen-binding fragment thereof is a camel antibody, or antigen-binding fragment thereof. In one embodiment, isolated antibody, or antigen-binding fragment thereof is a human antibody, or antigen-binding fragment thereof, is recombinant.

One aspect of the present invention is to provide screening methods for the detection and prognostic evaluation of cancer, for the identification of subjects possessing a predisposition to cancer, and for monitoring patients undergoing treatment of cancer as a surrogate marker of drug efficacy and for detecting recurrence, based on the detection of elevated levels of a Ciz1 autoantibody or circulating immune complexes (CIC) in biological samples of subjects. The term ‘autoantibody’ (or ‘autoantibodies’) is an antibody produced by the immune system of a subject that is directed against one or more of the subject's own proteins. The term ‘anti-Ciz1 autoantibody(ies)’ or ‘Ciz1 autoantibody(ies)’ refers to autoantibody(ies) specific for Ciz1. The invention also provides methods for detecting Ciz1 autoantibodies (whether free or in complex with Ciz1 antigen) as a diagnostic or prognostic indicator of cancer. In one embodiment the Ciz1 polypeptide is a Ciz1 b-variant polypeptide.

The present invention relates to diagnostic evaluation and/or prognosis of cancer by detecting a Ciz1 polypeptide or autoantibodies to a Ciz1 polypeptide antigen in a biological sample from a subject with cancer or at high risk for cancer (e.g., a smoker, patient with COPD, genetic predisposition for cancer). In one embodiment said biological sample assayed for anti-Ciz1 autoantibodies is selected from: blood, plasma, serum, sputum, urine or bronchoalveolar lavage. In another further embodiment the sample is blood. In another further embodiment the sample is plasma. In another further embodiment the sample is serum. In another further embodiment the sample is sputum. In another further embodiment the sample is urine. In another further embodiment the sample is bronchoalveolar lavage. In one embodiment the cancer is lung cancer, breast cancer, thyroid cancer, bladder cancer, liver cancer, kidney cancer, lymphomas and leukemias. In one embodiment the cancer is lung cancer. In a further embodiment the lung cancer is NSCLC. In another further embodiment the lung cancer is SCLC. In another embodiment the cancer is breast cancer. In another embodiment the cancer is thyroid cancer. In a further embodiment the thyroid cancer is medullary thyroid cancer. In another further embodiment the thyroid cancer is Hurthle cell carcinoma. In another further embodiment the thyroid cancer is papillary thyroid cancer. In another further embodiment the thyroid cancer is follicular thyroid cancer. In another embodiment the cancer is a lymphoma. In a further embodiment the lymphoma is B cell lymphoma. In another further embodiment the lymphoma is Hodgkin's lymphoma. In another further embodiment the lymphoma is diffuse large B-cell lymphoma. In another further embodiment the lymphoma is follicular lymphoma. In another further embodiment the lymphoma is anaplastic large cell lymphoma. In another further embodiment the lymphoma is extranodal marginal zone B-cell lymphoma. In another further embodiment the lymphoma is splenic marginal zone B-cell lymphoma. In another further embodiment the lymphoma is mantle cell lymphoma. In another embodiment the cancer is leukemia. In another further embodiment the leukemia is chronic lymphocytic leukemia. In another further embodiment the leukemia is hairy cell leukemia. The detection of increased levels of a Ciz1 polypeptide or autoantibodies to a Ciz1 polypeptide in the biological sample constitutes a novel strategy for screening, diagnosis and prognosis of cancer. In one embodiment the Ciz1 polypeptide is a Ciz1 b-variant polypeptide. In one embodiment the autoantibodies to the Ciz1 polypeptide are to a Ciz1 b-variant polypeptide. In one embodiment said autoantibody specifically binds to a contiguous epitope that includes amino acid residues encoded by both exon 14b and exon 15. In another embodiment said autoantibody specifically binds to a Ciz1 b-variant polypeptide but does not bind specifically bind a Ciz1 polypeptide translated from a mRNA comprising exon 14a. In another embodiment said autoantibody specifically binds to a Ciz1 b-variant polypeptide with an affinity at least 10 fold greater than to a Ciz1 polypeptide translated from a mRNA comprising exon 14a. In one embodiment said autoantibody binds with at least 10² fold greater affinity to a Ciz1 b-variant polypeptide as compared to a Ciz1 polypeptide translated from a mRNA comprising exon 14a. In one embodiment said autoantibody binds with at least 10³ greater affinity to a Ciz1 b-variant polypeptide as compared to a Ciz1 polypeptide translated from a mRNA comprising exon 14a. In one embodiment said autoantibody binds with at least 10⁴ greater affinity to a Ciz1 b-variant polypeptide as compared to a Ciz1 polypeptide translated from a mRNA comprising exon 14a. In one embodiment said autoantibody binds with at least 10⁵ greater affinity to a Ciz1 b-variant polypeptide as compared to a Ciz1 polypeptide translated from a mRNA comprising exon 14a.

In one embodiment said autoantibody specifically binds to the amino acid sequence DEEEIEVRSRDIS. In one embodiment said autoantibody specifically binds to the amino acid sequence DEEEIEVRSRDIS but does not specifically bind to the either the amino acid sequence DEEEIE, VRSRDIS or DEEEIEVEEELCKQVRSRDIS. In one embodiment said autoantibody specifically binds to the amino acid sequence EGDEEEEEDDEDEEEIEVRSRDISREEWKGSETY but not the amino acid sequence EGDEEEEEDDEDEEEIEVEEELCKQVRSRDISREEWKGSETY. In another embodiment said autoantibody specifically binds to the amino acid sequence DEEEEEDDEDEEEIEVRSRDISREEWKGSE but not the amino acid sequence DEEEEEDDEDEEEIEVEEELCKQVRSRDISREEWKGSE. In another embodiment said autoantibody specifically binds to the amino acid sequence DEEEEEDDEDEEEIEVRSRDISREEWKGSE but not the amino acid sequence DEEEEEDDEDEEEIEVEEELCKQVRSRDISREEWKGSE. In another embodiment said autoantibody specifically binds to the amino acid sequence EEEEDDEDEEEIEVRSRDISREEWKG but not the amino acid sequence EEEEDDEDEEEIEVEEELCKQVRSRDISREEWKG. In another embodiment said autoantibody specifically binds to the amino acid sequence EEDDEDEEEIEVRSRDISREEW but not the amino acid sequence EEDDEDEEEIEVEEELCKQVRSRDISREEW. In another embodiment said autoantibody specifically binds to the amino acid sequence DDEDEEEIEVRSRDISRE but not the amino acid sequence DDEDEEEIEVEEELCKQVRSRDISRE. In another embodiment said autoantibody specifically binds to the amino acid sequence DEDEEEIEVRSRD1SR but not the amino acid sequence DEDEEEIEVEEELCKQVRSRDISR. In another embodiment said autoantibody specifically binds to the amino acid sequence EDEEEIEVRSRDIS but not the amino acid sequence EDEEEIEVEEELCKQVRSRDIS. In another embodiment said autoantibody specifically binds to the amino acid sequence DEEEIEVRSRDI but not the amino acid sequence DEEEIEVEEELCKQVRSRDI. In another embodiment said autoantibody specifically binds to the amino acid sequence EIEVRSR but not the amino acid sequence EIEVEEELCKQVRSR.

The present invention provides for the use of a Ciz1 polypeptide or peptide thereof as an antigen in an immunoassay designed to detect the presence of autoantibodies to a Ciz1 polypeptide. Such immunoassays can be utilized for diagnosis and prognosis of cancer. In accordance with the invention, measurement of Ciz1 autoantibody levels in a subject's urine, blood, plasma or serum, etc. can be used for the early diagnosis of cancer. Moreover, the monitoring of autoantibody levels can be used prognostically to stage progression and recurrence of the disease.

The invention further relates to methods for detecting Ciz1 autoantibodies in a subject's biological sample. Such assays include immunoassays as described herein wherein the Ciz1 autoantibodiesdetected by their interaction with a polypeptide or peptide comprising a Ciz1 antigen. A Ciz1 antigen may be used to quantitatively detect the presence and amount of Ciz1 autoantibodies in a subject's biological sample.

The invention also relates to the use of polypeptide or peptide comprising a Ciz1 antigen to immunize a patient suffering from a disease characterized by increased expression levels of a Ciz1 polypeptide. Stimulation of an immunological response to such antigens, is intended to elicit a more effective attack on tumor cells; such as inter alia inhibiting tumor cell growth or facilitating the killing of tumor cells.

The invention further provides for pre-packaged diagnostic kits which can be conveniently used in clinical settings to diagnose patients having cancer or a predisposition to developing cancer. The kits can also be utilized to monitor the efficiency of agents used for treatment of cancer. In one embodiment of the invention, the kit comprises components for detecting and/or measuring the levels of autoantibodies directed toward Ciz1 polypeptide antigens in a sample. In a second embodiment, the kit of the invention comprises components which detect and/or measure Ciz1 polypeptide antigens in the biological sample.

In one aspect the invention provides for a method for diagnosis of cancer in a subject comprising: (a) quantitatively detecting levels of a Ciz1 polypeptide in a biological sample derived from a subject; (b) detecting levels of a Ciz1 polypeptide in a control sample; and (c) diagnosing the subject with cancer by comparing the levels of a Ciz1 polypeptide detected in the subject's sample to the levels of a Ciz1 polypeptide detected in the control sample, and identifying an increase in the levels of a Ciz1 polypeptide in the subject's sample, wherein an increase in the level of a Ciz1 polypeptide detected in the subject's sample as compared to a control sample is an indicator of a subject with cancer. In one embodiment the cancer is lung cancer. In another embodiment the cancer is SCLC. In one embodiment the Ciz1 polypeptide is detected using an immunoassay. In one embodiment the immunoassay is an immunoprecipitation assay. In one embodiment the biological sample is a lung tissue sample. In one embodiment the Ciz1 polypeptide is a Ciz1 b-variant polypeptide.

In one aspect the invention provides for a method for diagnosis of cancer in a subject comprising: (a) quantitatively detecting levels of Ciz1 autoantibodies in a biological sample derived from a subject; (b) detecting levels of a Ciz1 autoantibodies in a control sample; and (c) comparing the levels of Ciz1 autoantibodies detected in the subject's sample to the levels of a Ciz1 autoantibodies detected in the control sample, wherein an increase in the level Ciz1 autoantibodies detected in the subject's sample as compared to a control sample is an indicator of a subject with cancer. In one embodiment the cancer is lung cancer. In another embodiment the cancer is SCLC. In one embodiment the Ciz1 autoantibodies is detected using an immunoassay. In one embodiment the immunoassay is an immunoprecipitation assay. In one embodiment the sample is a lung tissue sample. In one embodiment the Ciz1 autoantibodies are autoantibodies to Ciz1 b-variant.

The present invention provides diagnostic and prognostic methods for diseases such as cancer based on detection of Ciz1 autoantibodies in a subject. The method may, e.g., be validated by the use of a biological sample from a subject with cancer and from age and gender matched controls, without cancer. A biological sample which may contain autoantibodies, such as urine, blood, serum or plasma, is obtained from a subject having or suspected of having a particular cancer or suspected of being predisposed to developing cancer. A corresponding body fluid may, e.g., be obtained from a subject that does not have cancer as a control.

In accordance with the invention, measurement of autoantibodies reactive against a Ciz1 polypeptide antigen can be used for the diagnosis of diseases such as cancer. Moreover, the monitoring of autoantibody levels can be used prognostically to stage the progression of the disease and for detection of recurrence. The detection of autoantibodies in a urine, blood, serum or plasma or other biological liquid sample from a subject can be accomplished by any of a number of methods. Such methods include immunoassays which include, but are not limited to, assay systems using techniques such as Western blots, radioimmunoassays, ELISA (enzyme linked immunosorbent assay), “sandwich” immunoassays, competitive immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays and flow cytometry to name but a few and including others disclosed elsewhere herein.

Such an immunoassay is carried out by a method comprising contacting a urine, blood, serum or plasma sample derived from a subject with a sample containing the a Ciz1 polypeptide antigen under conditions such that an immunospecific antigen-antibody binding can occur, and detecting or measuring the amount of any immunospecific binding by the autoantibody. The levels of autoantibodies in a urine, blood, serum or plasma sample may be compared to the levels present in an analogous biological sample from a subject not having the disorder, in a sample wherein the antigen is not present or wherein a different antigen is present.

The immunoassays can be carried out in a variety of ways. For example, one method involves immobilizing a Ciz1 polypeptide/peptide onto a solid support and detecting anti-Ciz1 antibodies specifically bound thereto. An alternative approach involves immobilizing autoantibodies from a biological sample, e.g., using an anti-human antibody or protein A or G, and detecting a Ciz1 polypeptide/peptide bound thereto, e.g., either by labelling the Ciz1 polypeptide/peptide or by detecting the Ciz1 polypeptide/peptide using an antibody or other appropriate means. The Ciz1 polypeptide/peptide antigen to be utilized in the assays of the invention can be prepared, e.g., via recombinant DNA techniques well known in the art or chemically synthesized. For example, a DNA molecule encoding a Ciz1 polypeptide or an antigenic fragment thereof can be genetically engineered into an appropriate expression vector for large scale preparation of a Ciz1 polypeptide. In other embodiments the Ciz1 antigen is engineered as a fusion protein that can facilitate labelling, immobilization or detection of the Ciz1 autoantibody. See, for example, the techniques described in Sambrook et al., 1989, Molecular Cloning: A laboratory Manual, Cold Spring Harbor Press, Cold Spring Harbor, N.Y. Alternatively, the a Ciz1 polypeptide may be purified from natural sources, e.g., purified from cells, using protein separation techniques well known in the art. Such purification techniques may include, but are not limited to molecular sieve chromatography and/or ion exchange chromatography. In practice, microtiter plates, beads or membranes are conveniently utilized as a solid support for the Ciz1 antigen. The surfaces may be prepared in advance and stored. In one embodiment the Ciz1 antigen is bound to a microtiter plate, in another beads, and in another a membrane. In another embodiment the binding of Ciz1 antigen is not bound to a solid support, such that binding of Ciz1 antigen to autoantibody take place in a liquid phase. In one embodiment Ciz1 antigen-autoantibody complex is detected using a labelled antigen binding molecule such as an antibody or aptamer. Preferably, the antigen binding agent is an antibody. The labelled antigen binding agent can be specific for either the Ciz1 antigen, e.g., in the case of liquid phase, or the autoantibody. In one embodiment the labelled antigen binding agent is an anti-human antibody antibody, i.e., an antibody specific for a human antibody. To facilitate binding of low affinity Ciz1 autoantibodies, the Ciz1 antigen may be multimerized into dimmers, trimers, tetramer, etc. In one embodiment the Ciz1 antigen is multimerized into tetramers using streptavidin (McLaughlin, K., et al. Protocol Exchange (Nature Publishing), published online 29 Jan. 2007).

In one embodiment a Ciz1 antigen used to detect Ciz1 autoantibodies comprises the amino acid sequence EGDEEEEEDDEDEEEIEVRSRDISREEWKGSETY. In one embodiment a polypeptide or peptide used to detect Ciz1 autoantibodies comprises the amino acid sequence EGDEEEEEDDEDEEEIEVRSRDISREEWKGSET. In one embodiment a polypeptide or peptide used to detect Ciz1 autoantibodies comprises the amino acid sequence DEEEEEDDEDEEEIEVRSRDISREEWKGSE. In one embodiment a polypeptide or peptide used to detect Ciz1 autoantibodies comprises the amino acid sequence EEEEDDEDEEEIEVRSRDISREEWKG. In one embodiment a polypeptide or peptide used to detect Ciz1 autoantibodies comprises the amino acid sequence EEDDEDEEEIEVRSRDISREEW. In one embodiment a polypeptide or peptide used to detect Ciz1 autoantibodies comprises the amino acid sequence DDEDEEEIEVRSRDISRE. In one embodiment a polypeptide or peptide used to detect Ciz1 autoantibodies comprises the amino acid sequence EDEEEIEVRSRDIS. In one embodiment a polypeptide or peptide used to detect Ciz1 autoantibodies comprises the amino acid sequence DEEEIEVRSRDI. In one embodiment a polypeptide or peptide used to detect Ciz1 autoantibodies comprises the amino acid sequence EEIEVRSR. In one embodiment a polypeptide or peptide used to detect Ciz1 autoantibodies comprises the amino acid sequence IEVRS. In one embodiment a polypeptide or peptide used to detect Ciz1 autoantibodies comprises the amino acid sequence EVRS.

In one embodiment a polypeptide or peptide used as a control in a method to detect Ciz1 autoantibodies comprises the amino acid sequence EGDEEEEEDDEDEEEIEVEEELCKQVRSRDISREEWKGSETY. In one embodiment the polypeptide or peptide control comprises the amino acid sequence EGDEEEEEDDEDEEEIEVEEELCKQVRSRDISREEWKGSET. In one embodiment the polypeptide or peptide control comprises the amino acid sequence EGDEEEEEDDEDEEEIEVEEELCKQVRSRDISREEWKGSET. In one embodiment the polypeptide or peptide control comprises the amino acid sequence DEEEEEDDEDEEEIEVEEELCKQVRSRDISREEWKGSE. In one embodiment the polypeptide or peptide control comprises the amino acid sequence EEEEDDEDEEEIEVEEELCKQVRSRDISREEWKG. In one embodiment the polypeptide or peptide control comprises the amino acid sequence EEDDEDEEEIEVEEELCKQVRSRDISREEW. In one embodiment the polypeptide or peptide control comprises the amino acid sequence DDEDEEEIEVEEELCKQVRSRDISRE. In one embodiment the polypeptide or peptide control comprises the amino acid sequence EDEEEIEVEEELCKQVRSRDIS. A Ciz1 polypeptide or peptide can also be used as a blocking agent in an assay to detect Ciz1 autoantibodies. In one embodiment a Ciz1 polypeptide or peptide used as a control in a method to detect Ciz1 autoantibodies comprises the amino acid sequence DEEEIEVEEELCKQVRSRDI. In another embodiment the polypeptide or peptide blocking agent comprises the amino acid sequence EGDEEEEEDDEDEEEIEVEEELCKQVRSRDISREEWKGSETY. In another embodiment the polypeptide or peptide blocking agent comprises the amino acid sequence EGDEEEEEDDEDEEEIEVEE ELCKQVRSRDISREEWKGSET. In another embodiment the polypeptide or peptide blocking agent the amino acid sequence EGDEEEEEDDEDEEEIEVEEELCKQVRSRDISREEWKGSET. In another embodiment the polypeptide or peptide blocking agent comprises the amino acid sequence DEEEEEDDEDEEEIEVEEELCKQVRSRDISREEWKGSE. In another embodiment the polypeptide or peptide blocking agent comprises the amino acid sequence EEEEDDEDEEEIEVEEELCKQVRSRDISREEWKG. In another embodiment the polypeptide or peptide blocking agent comprises the amino acid sequence EEDDEDEEEIEVEEELCKQVRSRDISREEW. In another embodiment the polypeptide or peptide blocking agent comprises the amino acid sequence DDEDEEEIEVEEELCKQVRSRDISRE. In another embodiment the polypeptide or peptide blocking agent comprises the amino acid sequence EDEEEIEVEEELCKQVRSRDIS. In another embodiment the polypeptide or peptide blocking agent comprises the amino acid sequence DEEEIEVEEELCKQVRSRDI. In another embodiment the polypeptide or peptide blocking agent comprises the amino acid sequence VEEELCKQV. In another embodiment the polypeptide or peptide blocking agent comprises the amino acid sequence EEELCKQ.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### DESCRIPTION OF THE FIGURES

FIG. 1 illustrates: a schematic representation of the Ciz1 gene showing exon structure. Regions that code for functional domains involved in DNA replication³, and attachment to the nuclear matrix¹ are indicated by black lines above. Dotted lines indicate uncertainty regarding domain boundaries. Gaps indicate sequences that are spliced out of variants with full activity in vitro. The location of PCR primers and probes are shown in relation to the known functional domains. Pink bar: probe T5 in exon 5, green bar: probe T7 at the junction between exons 6 and 7, yellow bar: probe T4 in exon 14, blue bar: probe T3 in exon 16. B) Quantification of Ciz1 expression (dCT values after normalization to actin), using the probes shown in A) across 46 cDNAs derived from lung carcinomas and normal adjacent tissues (Origene cDNA array HLRT504). Both reagent sets that amplify sequences within the Ciz1 replication domain (RD) generate a similar profile across the array. Conversely, reagent sets that amplify sequences in the nuclear matrix anchor domain (AD) generate a very similar profile to each other, but this is distinctly different to RD. C) Quantification of Ciz1 expression (dCT values after normalization to actin) in adjacent control tissues from 23 patients with stage IA, IB, IIA, IIB, IIIA, or IIIB tumours, and D) in the tumours themselves. Graphs include linear regression trend lines. E) To develop a single numerical indicator of the extent to which the balance between replication and anchor domain expression is altered in the tumor compared to matched control, RQ for the two replication domain probes or the two anchor domain probes (calibrated to the control tissues in sample set ½) were averaged. The average RQ for the tumour sample was divided by the average RQ for its matched control to give an individual measure of change relative to surrounding tissues for each domain. Values were combined by dividing the change in replication domain by the change in anchor domain so that, for example, increased expression of the replication domain that is balanced by increased expression of the anchor domain would result in a value close to 1. Conversely, increased expression of the replication domain that is exacerbated by decreased expression of the anchor domain would result in a value that is significantly greater than 1. Results are expressed on a log scale. Changes that are less than two-fold (indicated by grey region) are considered to be insignificant. This analysis does not reveal balanced under or over expression of Ciz1, and only reveals changes in expression relative to surrounding tissue. The degree of RD and AD imbalance increases with tumour stage;

FIG. 2 Uncoupled expression of DNA replication and nuclear matrix anchor domains in a range of solid tumours, as indicated. Histograms show relative quantification (RQ) of Ciz1 exon 7 (RD, white bars) and Ciz1 exon 16 (AD, black bars) in sample sets represented in cDNA array CSRT1. For each tissue type, analysis of 9 independent tumours of increasing stage (left to right) are shown alongside 3 unmatched control samples derived from apparently normal tissue from cancer patients (identified as control). Results for the two probes were normalized to an average of the controls (C) shaded in grey, for RD, so that RQ=2^{-(Ct exon test-Ct ex 7 average control)}Results for lung tumours stages Ito III are comparable to the sample set analysed in FIG. 1 and are shaded in grey. For all tumour types examples of stage IV tumours are also included. For most of these expression of RD is equal to or exceeds RD (indicated with an *). B) Right panels show the ratio of expression of AD and RD (Ratio=Ct exon 16/Ct exon7) with increasing stage from left to right. The first data point represents the averaged control and the last data point a stage IV sample. Quadratic regression trend lines were generated using excel. For all tumour types except liver, the trend shows a proportional increase in AD relative to RD in early stage tumours compared to controls and a reversal of this trend at later stages, so that for most stage IV tumours RD often exceeds AD;

FIG. 3 A) Analysis as in FIG. 2, indicates altered expression in favour of AD in 40 malignant melanomas compared to control samples. Results for the two sets of detection tools are normalized to 1 for the first control sample. Right panel, summary of results for stage II, III and IV tumours indicating the % of samples in which anchor domain expression exceeds that of the replication domain;

FIG. 4 A) Ways in which uncoupled expression of Ciz1 replication domain (black line) and nuclear matrix anchor domain (yellow circle) could influence immobilization of Ciz1 and the sub-nuclear localization of its DNA replication activity. Grey barrels represent DNA replication proteins assembled at replication origins, grey ovals represent nuclear matrix-associated docking sites for Ciz1. The model assumes that nuclear matrix-associated docking sites are limiting. Right panel shows a variant of Ciz1 with impaired ability to become assembled into the nuclear matrix. B) Summary of two types of Ciz1 mis-expression seen in human tumours. i) Uncoupled expression as seen in most common solid tumours and described in FIGS. 1-3, ii) b-variant as seen in a high proportion of small cell lung cancers, thyroid cancers and lymphomas;

FIG. 5. Generation and Validation of Ciz1 replication domain (RD) and anchor domain (AD) antibodies, and analysis of RD and AD protein expression. A) Schematic representation of Ciz1 exons (shaded rectangles) showing the regions used as immunogen for polyclonal antibodies (upper panel) and monoclonal antibodies (lower panel). B) Representative immuno-fluorescence images of endogenous Ciz1 detected with Ciz1-RD antibody (red) in normal fetal lung cells (WI38) and two representative neoplastic cell lines as indicated, without treatment prior to fixation (‘unextracted’), after extraction of soluble proteins in the presence of 0.1% triton X100 (‘detergent resistant’) and after incubation with DNAse 1 (‘DNase resistant’). Images were collected under identical conditions with standardized exposure times, so that within and between cell lines the intensity of Ciz1 and of DNA reflects the level of Ciz1 and DNA remaining in the cell under the different conditions. Total DNA is stained with Hoechst 33258 (blue). Bar is 10 microns. Similar results were obtained for four other cancer cell lines of different origins. C) As in B, except that detection is with Ciz1-AD antibody (green). Results illustrate i) uncoupled and imbalanced expression of Ciz1-RD and Ciz1-AD at the protein level, ii) elevated Ciz1-RD protein that is not immobilized in cancer cells, iii) Immobilization of the majority of Ciz1-AD protein; D) Effect of recombinant AD protein on immobilization of endogenous Ciz1. High-magnification images of the DNAse-resistant fraction of endogenous Ciz1-RD (red) in NIH3T3 cells without (left panel) or with (right panels) expression of recombinant GFP-C275 (green), which encodes murine AD protein. Total DNA is stained with Hoechst 33258 (blue). Note the reduced focal staining in cells transfected with GFP-C275. E) Images show NIH3T3 nuclei with focal pattern of GFP-Ciz1, non-focal pattern of GFP-C275 and cells co-transfected with both vectors, after extraction with detergent. Green is GFP, blue shows nuclei stained with Hoecsht 33258. GFP-C275 interferes with the formation of GFP-Ciz1 subnuclear foci.

FIG. 6 A) Scheme indicating the products generated using b-type transcript junction spanning primer (red arrow) and the location of junction-spanning taqman probe (red line). B) Mobility variation observed in cloned products with b-variant exon from a SCLC cell line and full length products from a normal cell line. C) Junction-spanning primer was verified using reporter plasmids expressing normal transcript (clone 19) or b-type transcript (clone 20). Gels show plasmid derived PCR products from selective primer pair P3/4 or unselective Ciz1 primer pair P1/2. D) PCR products generated from cDNA prepared from 2 neuroendocrine lung cancer cell lines (L95, SBC5) and one normal fetal lung cell line (HFL1) using primer set P11/P12 (actin, lower panel), primer set P1/P2 (Ciz1, upper panel), or b-type transcript junction-spanning primer set P4/P3 (middle panel). Products were sequence verified, noT is a no template control lane. E) Primers from either side of the variable region (P1/P2 or P6/P7) were coupled with taqman probes that either span the unique junction in b-type transcript (T2) or which recognise a region that is not alternatively spliced (T4 and T3). Application to mixtures of plasmid clones 19 and 20, that contained either 100, 75, 50, 25, or 0% clone 20, demonstrate selective detection of b-type transcripts. Graph shows that cycle number required to reach the threshold is constant for un-selective detection tools, but affected by plasmid mixture composition for variant-selective tools;

FIG. 7 A) QPCR for RD (left panel) or AD (centre panel) as in FIGS. 1-3, of b-variant using templates from three ‘normal’ embryonic lung cell lines and three neuroendocrine lung tumour cell lines, plus one neuroendocrine carcinoid. Results are normalized to actin and calibrated to IMR90 RD. D) Human lung cancer tissues. The same detection tools were applied to cDNA from 3 SCLC patients and three normal adjacent tissue from the same individuals. Expression of b-type transcript is dramatically elevated in these neuroendocrine tumours;

FIG. 8 A) Expression of b-type transcripts (black bars) in matched sample sets from 23 lung cancer patients (same sets as FIG. 1) ranging from grade I to grade III (Origene cDNA array HLRT504). Expression is normalized to actin and expressed relative to the ‘normal’ sample (white bars) in each pair, which is given an arbitrary value of 1. B) Similar analysis of a separate set of non-small cell lung tumours and unmatched controls from the stages indicated (Origene array CSRT303). Histogram shows b-variant RQ after normalization to actin. C) Comparable results for liver tumours and D) kidney tumours also derived from CSRT303. Results are calibrated to an average of the control tissues samples, indicated by a grey block. For all sample sets shown in FIG. 8, b-variant is elevated in a small number of random cases;

FIG. 9 illustrates analysis as in FIG. 8 for lymphoma, thyroid, bladder, liver and kidney cancers.

FIG. 10 Generation and Validation of exon 14b-variant protein detection tools. A) Immunogenic peptide lacking intervening sequence (grey) to generate unique EEIEVRSR junction within a 16 amino-acid peptide (lower line), and full length peptide used to remove antibody species that react with junction flanking epitopes (upper line). Polyclonal sera and hybridomas were negatively screened against immobilized full-legnth peptide and positively selected or affinity purified using 14b junction containing peptide to generate affinity purified polyclonal antibody (antibody 2B). B) Immuno-fluourescence with anti-b-variant antibody using NIH3T3 cells expressing GFP-hCiz1 or GFP-hCiz1 b-variant (green). Recombinant 14b protein is detected in red, and DNA is stained in blue. C) Western blots showing selective detection of over-expressed GFP-Ciz1 protein harboring the 14b exon junction. Results with anti-b-variant serum, pre-immune serum and anti-Ciz1 polyclonal antibody are shown. D) Immuno-detection of endogenous 14b protein with affinity purified anti-b-variant polyclonal antibody in SCLC cells and representative normal cells as indicated. SCLC cells react with anti-b-variant serum but normal cells do not. E) Detection of Ciz1 in the same cells is shown for comparison. F) High-magnification (600×) images of SCLC cells as in D, revealing discrete foci in the nucleus that are similar in size but fewer in number than DNA replication foci.

FIG. 11 Development of b-type transcript selective RNA interference tools. A) Top panel, schematic showing a panel of siRNA sequences spanning the unique exon junction. Lower panels show their effect on Ciz1 AD transcript levels and b-type transcript levels, 24 hours after transient transfection into SCLC cells. Results are normalised to actin and calibrated to samples from cells transfected with control siRNA (Dcon). B) Results are expressed as a ratio of AD to b-type transcript, where control siRNA has a ratio of 1. The most effective and selective siRNA sequence was chosen for further testing (starred) C). Variant-selective effect on expression of recombinant Ciz1 protein. Clones 19 and 20 were co-transfected with b-type transcript selective siRNA or control siRNA as indicated, into mouse 3T3 cells. B-type transcript siRNA suppresses expression of protein from expression clone 20, but not endogenous mouse Ciz1 or human Ciz1 from expression clone 19;

FIG. 12 Effect of inducible expression of b-variant selective shRNA on SCLC cell proliferation in culture. A) Stable expression of the chosen b-type selective sequence and a control sequence (against luciferase) from a dox-regulated shRNA vector (Clonetech). Results show increase in cell number over 4 days. Dox was added to test samples at 0 and 3 days (black arrow heads). Control cells (SCLC expressing luciferase shRNA) are largely unaffected by induction while test cells (SCLC expressing b-type selective sequence) are prevented from proliferating at the normal rate. B) An independent experiment in which doxycyclin was added at day 0 and cell number was quantified in triplicate at 4 days. Error bars show SEM. C) Gel images show RT-PCR products and the selectivity of the chosen sequence for b-type transcripts versus total Ciz1 expression. By 26 hours after induction b-type transcript levels have recovered, while a second dose one hour before samples were isolated reveals selective suppression of b-type transcripts. D) Suppression of b-variant protein in SBC5 cells, detected with b-variant polyclonal antibody 48 hours after induction of shRNA expression with doxycyclin. E) SBC5 harbouring inducible b-variant shRNA vector cells after 1 month in culture in low tet serum without induction. Chronic leaky expression has visible and progressive effects on cells;

FIG. 13 In vivo study (Southern Research Institute, USA). A) Two cohorts of 15 NOD/SCID mice were injected with 1.5×107 cells harbouring dox-regulated b-type variant selective shRNA vector on day 0. At 21 days mice with tumours less than 100 mg were discounted creating groups with equal mean tumour weight and low inherent variation. Dox was administered in drinking water to group 2 (black circles) at 21 days and tumour size was measured twice weekly thereafter. Graphs show mean tumour weight with SEM B) An additional 10 mice were maintained on Dox from 3 days prior to injection with SCLC cells. Results show their mean tumour weight with SEM, compared to mean tumour weight of 15 mice that did not receive dox. C) Quantitative RT-PCR showing the relative levels of b-type transcript in whole blood-derived cDNA of two mice with tumours from group 1 (open circles in FIG. 14A) and two mice without tumours from group 3 (closed squares in FIG. 14B). Histogram shows duplicate analyses (each is average of triplicate samples) after normalization to murine actin, and calibration to sample SRI-3-8. Estimated size of subcutaneous tumour carried by the four mice is also shown.

FIG. 17 A) Schematic representation of the Ciz1 gene showing exons (numbered), and the location of siRNAs (grey triangles). B) Suppression of human Ciz1 transcript following transient transfection of human SCLC cell line SBC5 with Dharmacon smart pool anti-human Ciz1 siRNAs, individually (A, B, C, D) or as a mixture, and with Dharmacon smart pool control siRNA. Histogram shows relative quantification (RQ) of Ciz1 anchor domain transcript at the indicated times, detected with primers P1/P2 and probe T4. Results are normalized to actin and calibrated to the result for cells transfected with control siRNA, which is given an arbitrary value of 1. C) Effect of siRNA B and control siRNA on Ciz1 protein in western blots of detergent-soluble supernatant (SN) and detergent-resistant pellet (P) protein fractions from SBC5 cells, harvested 24 hours after transfection. Ciz1 protein was detected with anti-mouse Ciz1 RD polyclonal antibody 1793. Multiple Ciz1 isoforms are detected as reported previously for NIH3T3 cells and U20S cells. D) Effect of anti-human Ciz1 siRNA B (grey squares), and Ciz1 siRNA 1 (grey circles), Ciz1 siRNA 3 (grey triangles), and control siRNA (open circles) on proliferation of SBC5 cells over 5 days following a single transient transfection. Results are expressed as fold increase in cell number over day 1, with SD derived from three independent populations.

Applicant has made the discovery that, in addition to solid tumor samples, Ciz1 b-variant polypeptide can be detected in the plasma of cancer patients. This finding is remarkable and unexpected because Ciz1 is a nuclear protein and is not known to be secreted. Moreover, proteases are present in blood that degrade many proteins. Even more unexpectedly, applicant has discovered Ciz1 b-variant polypeptide in the plasma of early stage cancer patients (stage 1 NSCLC and limited stage SCLC) when tumor burden is low. The Ciz1 b-variant biomarker detects cancer both a high degree of sensitivity and specificity.

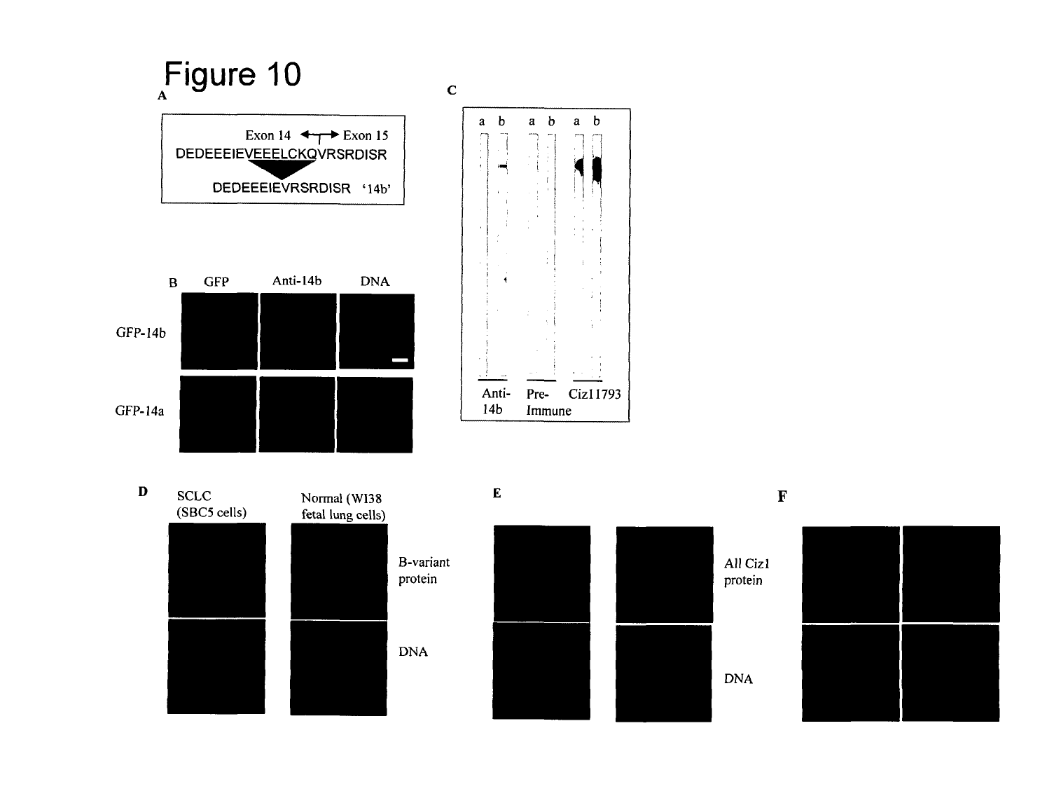

FIG. 18 B-variant Ciz1 protein in lung cancer patient plasma. A) Ciz1 gene showing exons (numbered), the DNA replication domain and nuclear matrix anchor domain witha representation of Ciz1 b-variant, which lacks part of exon 14 directly below. B) Western blot showing b-variant protein in 1 μl of plasma from patients with SCLC and NSCLC, plus 5 samples from individuals with no diagnosed disease, detected with antibody 2B (described in Supplementary FIG. 10). Endogenous immunoglobulin is used to normalize for loading (control). C) Mean b-variant protein levels (with SEM), determined by densitometry of western blots, showing results for a total of 119 pre-treatment samples from lung cancer patients with the indicated type and stage of disease, plus 51 samples from individuals with no disease, or patients with chronic obstructive pulmonary disease (COPD), asthma or anaemia. Using a threshold set at the mean of the non-cancer samples (+1 SD), the test correctly classified 93% of limited stage SCLC and stage 1 NSCLC patients. D) Receiver operating characteristic curve, with 95% confidence interval, generated for all 170 samples using a web-based calculator for ROC analysis of continuously distributed data (AUC is 0.958). A web based calculator available at http://www.jrocfit.org (format 5 for continuously distributed data) was used for the calculation.

TABLE 1
Summary of oligonucleotide primers and probes.

| Desig- | | |
|---|---|---|
| nation | Sequence | Exon |
| Primers | | |
| P1 | CAGGGGCATAAGGACAAAG | 13F |
| P2 | TCCGAGCCCTTCCACTCCTCTCTGG | 15R |
| P3 | TCAGGTTTTGAGGCGGGTTGAG | 17R |
| P3′ | GGTTTTGAGGCGGGTTGAG | 17R |
| P4 | GAAGAGATCGAGGTGAGGTC | 14bF |
| 6 | CGAGGGTGATGAAGAAGAGGA | 14F |
| 7 | CCCCTGAGTTGCTGTGATA | 16R |
| 9 | CACAACTGGCCACTCCAAAT | 5F |
| 10 | CCTCTACCACCCCCAATCG | 5R |
| P11 | CAACCGCGAGAAGATGACC | Ac- |
| | | tin F |
| P12 | TCCAGGGCGACGTAGCACA | Ac- |
| | | tin R |
| 13 | ACACACCAGAAGACCAAGATTTACC | 6/7 |
| | | junc- |
| | | tion |
| 14 | TGCTGGAGTGCGTTTTTCCT | 7 |
| P3b | GAA TCT CCA GGG CAC CAA C | 3F |
| P5 | CGA TTG GGG GTG GTA GAG G | 5R |
| P24 | TGTTGCATGAGAAAACGCCA | Albu- |
| | | min F |
| P25 | GTC GCC TGT TCA CCA AGG AT | Albu- |
| | | min R |
| Probes | | |
| T1 | CACTGCAAGTCCCTGGGCCA | 16 |
| T2 | TGGACCTCACCTCGATCTCTTCTTCA | 14b |
| T3 | CACGGGCACCAGGAAGTCCA | 16 |
| T4 | TGGTCCTCATCTTGGCCAGCA | 14 |
| T5 | CGCCAGTCCTTGCTGGGACC | 5 |
| T6 | CCCTGTACGCCTCTGGCCGT | Ac- |
| | | tin |
| T7 | ccc tgc cca gag gac atc gcc | 7 |
| T26 | AAG TGA CAG AGT CAC CAA ATG CTG CA | Albu- |
| | | min |

### EXAMPLES

### cDNA arrays

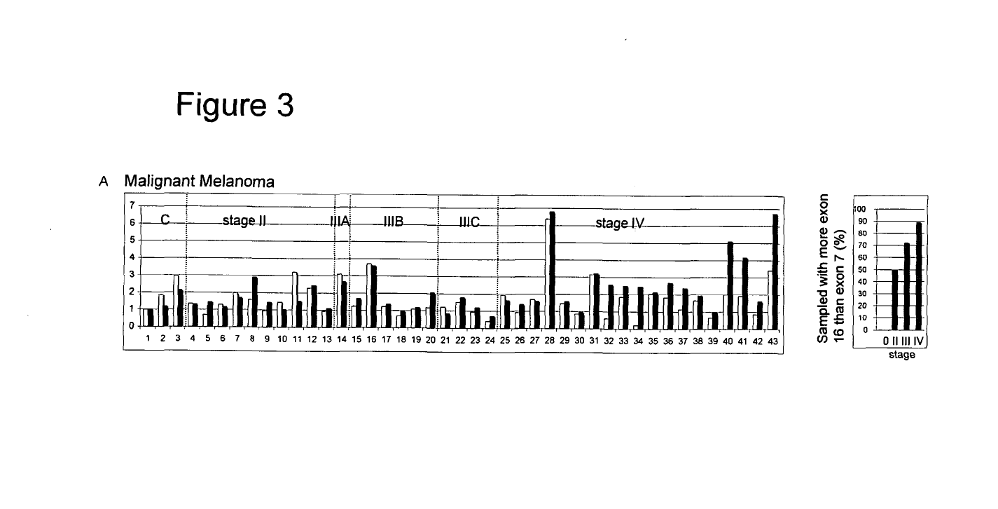

TissueScan qPCR arrays containing 2-3 ng of cDNA from 48 different lung samples (HLRT101), and 24 matched pairs of lung carcinoma and adjacent tissue from the same patient (HLRT504), or 10 sets of tissue samples from different cancers (CSRT504) were from OriGene Technologies, Inc. (Rockville, Md.). Tumour classifications and abstracted pathology reports for the lung/normal matched pair tissue array are as given at http://www.oriaene.com/geneexpression/disease-panels/products/HLRT504.aspx. The level of cDNA in each well was standardized for *b-actin expression by the supplier and amplification of *b-actin to normalize results for Ciz1 expression, in multiplex reactions for the data in FIG. 3B and single reactions for all other arrays. Thresholds were set and all analysis performed using ABI 700 software.

Human tissue derived RNA. Three pairs of lung tumour/normal RNA from tissues collected under IRB approved protocols, were from Cytomyx (http://www.cvtomyx.com/cytomyx/cytomyx_biorepository.asp). Additional samples of human lung tissue, collected with informed donor consent, were obtained from ILSbio (http://www.ilsbio.com/). RNA was isolated from tissues using TRIzol according to manufacturers instructions; tissue homogenisation was carried out using an RNase free 1.5 mL Pellet Pestle (Anachem). RNA samples were reverse transcribed with random primers, or a mixture of oligo dT and random primers as follows. Approximately 1.6 *mg of total RNA was incubated with 1 μL 10 mM dNTPs, 0.5 μL 0.5 μg/μL random primers (Promega) and 0.5 μL 0.5 μg/μL oligo dT₁₂₋₁₈ Primer (Invitrogen) to a total volume of 12 μL in DEPC water. Alternatively, total RNA was incubated with 1 μL 500 μg/mL random primers, 1 μL 10 mM dNTPs to a total volume of 13 μL in DEPC water. Samples were incubated at 65° C. for 10 minutes in a PTC-200 Peltier Thermal Cycler (MJ Research), followed by incubation on ice for 5 minutes. To the random primed reactions the following were added to a volume of 20 μL: 1× First-Strand buffer, 5 mM DTT, 200 U SuperScript III and 40 U RNaseOUT (all Invitrogen). Reactions were incubated at 46° C. for 3 hours, followed by 70° C. for 15 minutes. To the random primer/oligo dT reactions the following were added in a final volume of 20 μL: 1×M-MLV reaction buffer, 10 mM DTT, 200 U M-MLV reverse transcriptase (all Promega) and 40 U RNaseOUT (Invitrogen). Reactions were incubated at 42° C. for 52 minutes, followed by 70° C. for 15 minutes.

PCR and QPCR

Primer pair combination used for fragment amplification included p8/p2 using Taq polymerase (NEB, Herts, UK), 94° C./5 minutes and then 33 cycles of 94° C./15 seconds, 55° C./30 seconds and 68° C. for 1 minute, and a final step at 68° C. for 7 minutes), p1/p2 using phusion polymerase (Finnzymes, Espoo, Finland) 98° C./30 seconds and 33 cycles of 98° C./10 seconds, 62° C./30 seconds and 72° C. for 40 sec, and 72/° C. for 7 minutes and p4/p3 using Taq olymerase(NEB, Herts, UK), 94° C./5 minutes and then 33 cycles of 94° C./30 seconds, 62° C./30 seconds and 72° C./40 seconds followed by a final step at 72° C. for 7 minutes). PCR reactions were run on an MJ thermal cycler PTC-200. Quantitative PCR reactions were carried out in MicroAmp™ optical 96-well reaction plates with optical adhesive film (Applied Biosystems) in a total volume of 25 μL. For each reaction cDNA was incubated with 1× TaqMan® PCR mix (Applied Biosystems), 0.4 μM forward primer, 0.4 μM reverse primer and 0.4 μM probe. Samples were run on the ABI Prism 7000 or 7300 Sequence Detection system using the relative quantification assay, and the following programme; 50° C. [2 minutes], 95° C. [10 minutes], followed by 40 cycles of 95° C. denaturation [15 seconds], 60° C. annealing and elongation [1 minute]. The cycle number at which the sample passed the threshold level is the Ct value. One sample was selected as the ‘calibrator’ sample and all other expression values expressed relative to it (RQ). Unless stated otherwise primers were from Sigma Aldrich, probes were from MWG, and sequence verification of clones and PCR products was carried out by MWG.

Cell Culture and Transfection

Cell lines were obtained from the European cell culture collection (http://www.ecacc.org.uk/) or the Japanese Collection of Research Bioresource (http://cellbank.nibio.go.jp/), or were a kind gift from J. Southgate. All cell lines were cultured as recommended. NIH3T3 cells were grown as previously described and transfected with GFP-Ciz1 or GFP-C275, using Mirus 3T3.

Nuclear Fractionation

Nuclear fractionation was essentially as described. Typically cells on coverslips were rinsed with cold PBS, then cold CSK buffer (10 mM Pipes/KOH Ph6.8, 100 mM NaCl, 1 mM EGTA, 300 mM sucrose) plus 1 mM DTT, and protease inhibitor cocktail (Roche), with our without detergent (0.1% TX100) as indicated. For DNase treatment cells were further rinsed in CSK (0.1 or 0.5M NaCl as indicated), followed by PBS, followed by incubation with DNase 1 in digestion buffer (10 mM Tris [pH 7.6], 2.5 mM MgCl₂, 0.5 mM CaCl₂) at 25° C. for 20 minutes, as recommended (Roche). Where indicated DNAse treated cells were rinsed with 0.5M NaCl for 1 minute prior to fixation. All preparations were fixed with fresh 4% paraformaldehyde for 20 minutes at room temperature,.

Immunofluorescence

Fixed cells on coverslips were washed with PBS then blocked with antibody buffer (10% protease-free BSA, 0.02% SDS, 0.1% Triton X-100 in PBS). Ciz1-RD was detected with anti-Ciz1 polyclonal antibody 1793 and Ciz1-AD with polyclonal antibody 2C affinity purified using Ciz 1 anchor domain peptide DEDEEEIEVEEELCKQVRSRDISR. DNA was counterstained with Hoechst 33258 (Sigma). Images were collected using a Zeiss Axiovert 200 M and Openlab image acquisition software, using identical exposure parameters within an experiment, typically 300 ms for TRITC-labelled Ciz1, 400 ms for GFP, 15 ms for Hoescht. Where images were digitally enhanced to remove background fluorescence or increase brightness using Adobe photoshop, identical manipulations were applied to images within one experiment. So, for example the intensity of Ciz1 staining before and after extraction reflects the effect of the treatment. Fluorescence intensity was quantified from raw images acquired under identical imaging parameters using the Openlab ‘Profile’ tool.

### Example 1

### Uncoupled Expression of DNA Replication and Anchor Domains

The two well-characterized functions of Ciz1 (cyclin-dependent stimulation of DNA replication and association with the nuclear matrix) are encoded by separate protein domains. These are called RD (replication domain) and AD (anchor domain). In vitro, Ciz1 does not require its nuclear matrix anchor in order to promote DNA replication. In fact Ciz1 fragments lacking AD appear to be more active than those that would be attached to the nuclear matrix³, implying that immobilization is a constraining feature rather than one that is intrinsic to function. Here, presented is evidence that expression of RD and AD are not coincident in most cancer cells, i.e., “uncoupled expression”. Expression of one or other of the domains is altered and imbalanced in the majority of lung cancers, as well as a range of other common solid tumours.

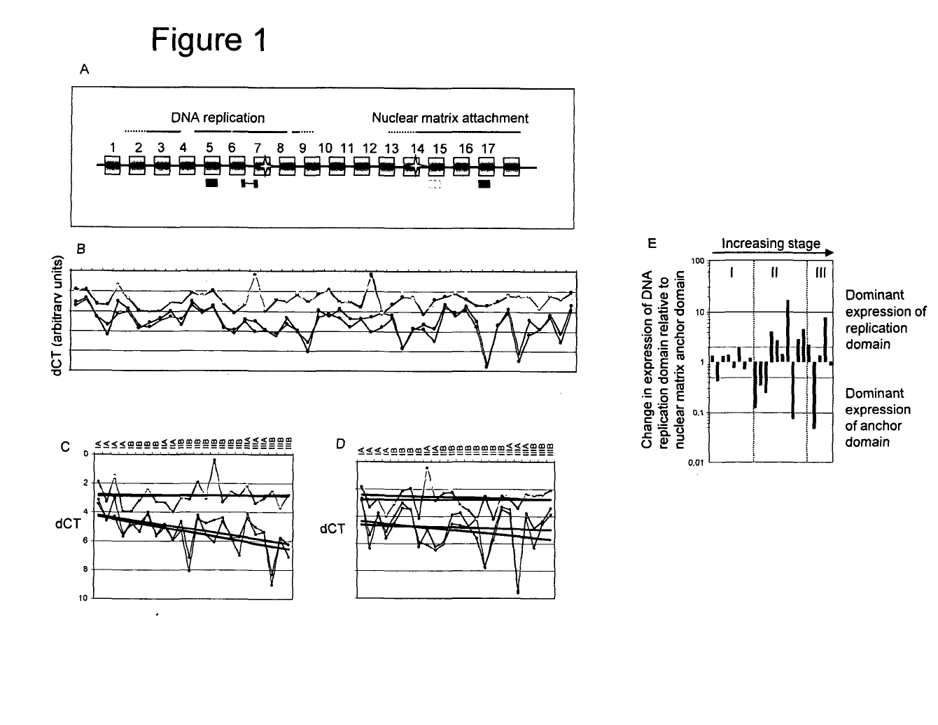

Quantitative PCR reagents (FIG. 1a) that detect expression of RD or AD were used to interrogate a cDNA array that contains 46 lung-derived cDNAs (FIG. 1b). Across the array, both RD probes revealed a consistent pattern of expression. Similarly, both AD probes revealed a consistent pattern of expression. However, expression of RD and AD are far from identical to one another. This demonstrates that the two domains are not always expressed together, and that they are probably not always both present in Ciz1 protein.

Uncoupled Expression in Lung Tumours

In contrast to the adjacent control samples, the tumours themselves exhibit a far less convincing trend. Although Ciz1 expression is clearly uncoupled and imbalanced, for some patients this is manifest as decreased RD and for others as increased RD (relative to the stage IA samples), giving rise to a near horizontal trend line with poor fit.

The combined effect of increased expression of one domain and decreased expression of the other is also revealing. When the combined results for RD and AD expression is presented relative to each individual adjacent control (FIG. 1E), the data show that disruption of their balance ratio correlates with tumour stage. For tumours from patients with stage 1 disease, 12.5% (1 of 8) have a greater than two-fold change in the balance between AD and RD compared to surrounding tissue, while for stage II tumours this is 90% (9/10), and for stage III tumours 60% (3/5). This trend supports the conclusion that Ciz1 expression is uncoupled and unbalanced during tumourigenesis.

Uncoupled Expression in Other Types of Tumour

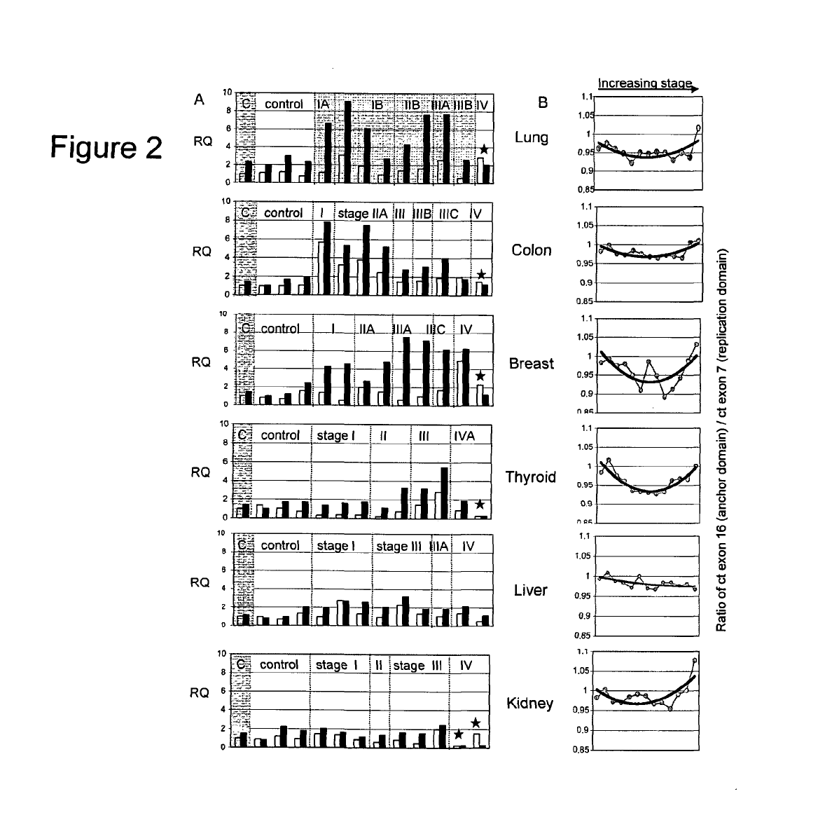

To generate an overview of Ciz1 transcript expression, RD and AD were sampled in a number of common solid tumours (FIG. 2). AD is over-represented in almost all stage I, II and III tumours relative to the (unmatched) control samples for most tumour types. This is most apparent for breast, lung and thyroid cancers (evident from the dip in the ratio curves shown in FIG. 2B).

Uncoupled Expression in Stage IV Disease

Notably, in more than half of the stage IV tumours from all tissues types the reverse applies (indicated with asterisk in FIG. 2A). In these samples RD transcript is over-represented, suggesting that expression is disrupted in favour of RD in a subset of tumours that have undergone or will undergo metastasis.

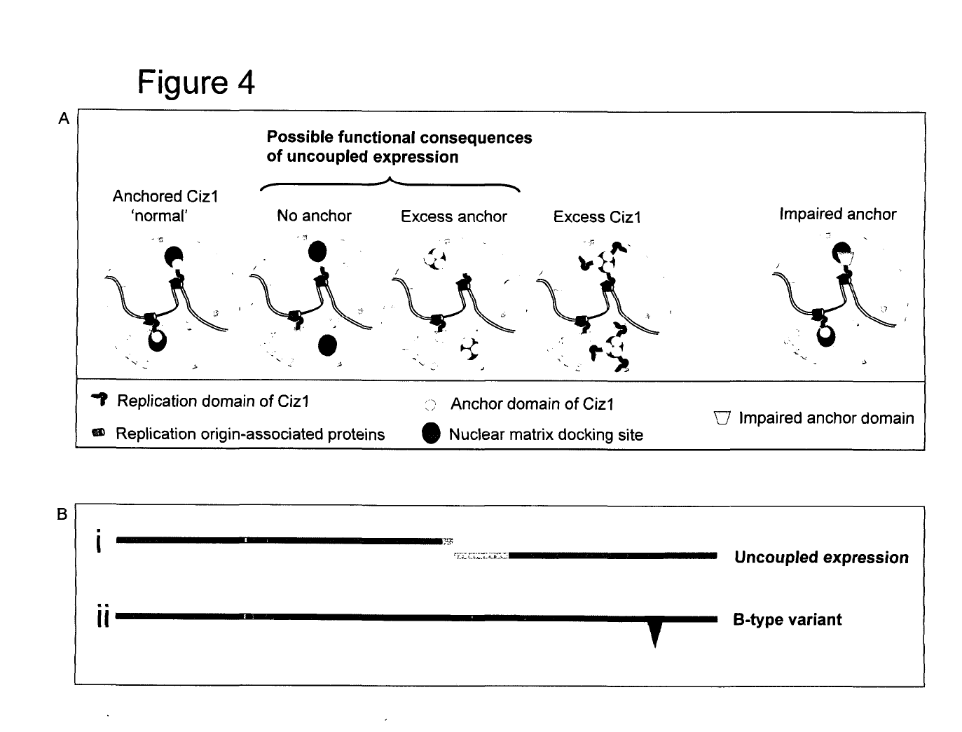

A similar analysis was applied to 40 malignant melanoma samples, including 19 samples from patients with stage IV disease (FIG. 3A). In the majority of tumours of all grades AD expression exceeds RD, while for all three control samples this is not the case. Therefore, malignant melanomas do not follow the trend described above, indicating that a switch to dominant expression of RD does not accompany metastatic capability for this type of tumour. When considered at the protein level and in the light of what is already known about Ciz1 function, the impact of excess RD or excess AD on cellular DNA replication could be very similar, with possible differences in severity. Specifically, it was known by applicant that the replication domain of Ciz1 is capable of functioning to stimulate initiation of DNA replication in the absence of its nuclear matrix anchor³, but that nuclear matrix attachment is the norm for the majority of Ciz1 in NIH3T3 cells¹, and most other established cell lines of non-tumour origin that applicant has tested (not shown). Applicant suggests that expression of the replication domain in the absence of its nuclear matrix anchor would result in unanchored activity, and that this would have a consequence for the spatio-temporal organization of DNA replication. Similarly, expression of C-terminal immobilization domains in the context of a protein that does not possess catalytic function could have a dominant negative effect by competing with full-length protein for immobilization sites on the nuclear matrix (FIG. 4A).

### Example 2

### Protein Detection Tools

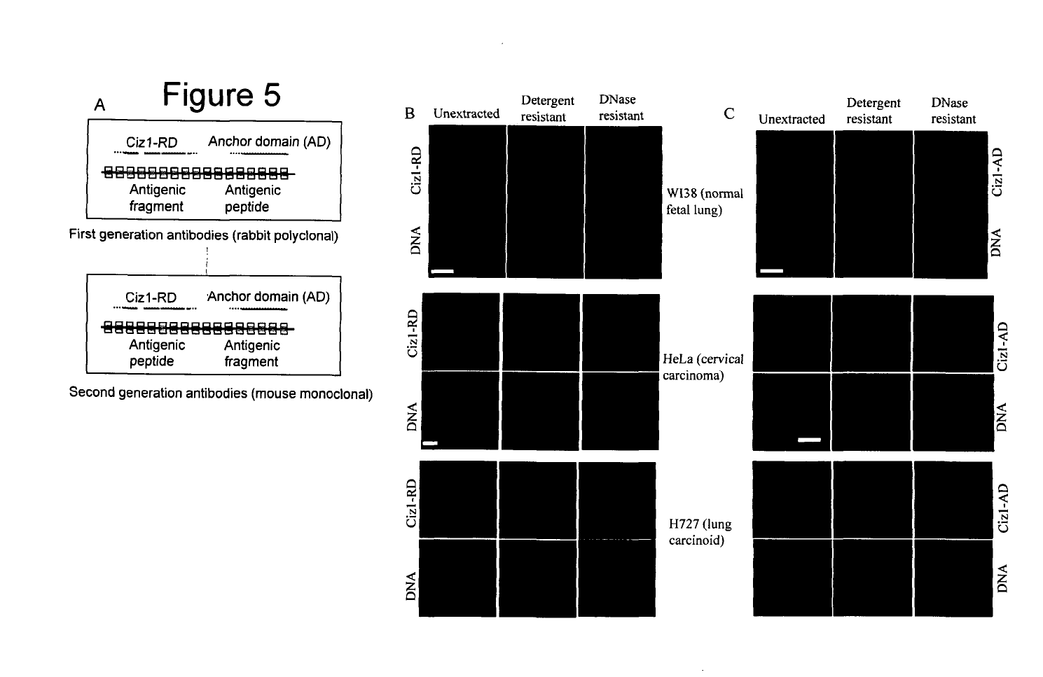

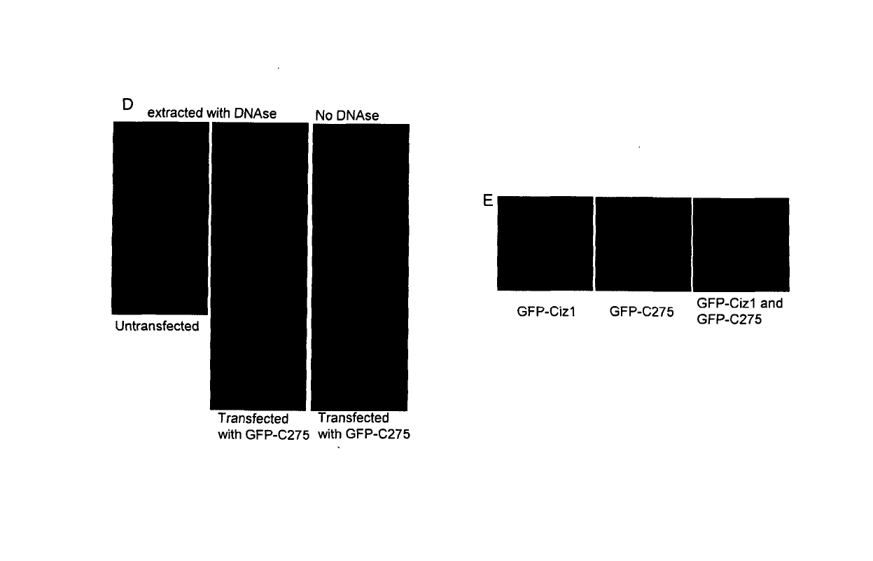

Applicant has developed a set of monoclonal and polyclonal antibodies against RD and AD (FIG. 5A), with which to detected Ciz1 expression at the protein level. These have potential as molecular diagnostic tools, and are currently being used to answer questions about Ciz1 protein function and behaviour in cancer cell lines. So far applicant has demonstrated that Ciz1 RD and AD both exist independently at the protein level (FIG. 5B,C), that AD is attached to the nuclear matrix in some cancer cells in which RD is not (FIG. 5C), and that over expression of AD disrupts the normal sub-cellular localization and immobilization of endogenous RD (FIG. 5D,E). All of these observations are consistent with the idea that disruption of the ratio between Ciz1 RD and AD alters the architecture of the nucleus.

### Example 3

### B-type Variant

Applicant surveyed expressed sequence tags (ESTs) that map to the Ciz1 Unigene cluster Hs. 212395 (http://www.ncbi.nlm.nih.qov/sites/entrez?db=unigene) for evidence of alternative splicing in the Ciz1 coding sequence. This suggested that neuroendocrine lung cancers (primarily small cell lung cancers, SCLC) express a form of Ciz1 that is alternatively spliced (to yield b-type transcripts), far more frequently than non-cancer tissues (illustrated in FIG. 4B). Ciz1 transcripts that span the region that is alternatively spliced in b-type transcripts were detected in a total of 23 different libraries, 10 carcinomas and 13 non-carcinomas. For the carcinoma-derived transcripts 40% were b-type transcripts, compared to only 3% from non-cancer libraries.

Selective Detection Tools

Applicant developed molecular tools that detect b-type transcripts. These are primers located either side of the exon junction, a primer that spans the exon junction and only gives a product from b-type transcripts, and a Q-PCR probe that also spans the exon junction and only recognizes b-type transcripts. Initially these were applied to a panel of lung cancer cell lines to a) validate the tools and b) generate confirmatory data on expression of b-type transcripts.

Expression in SCLC

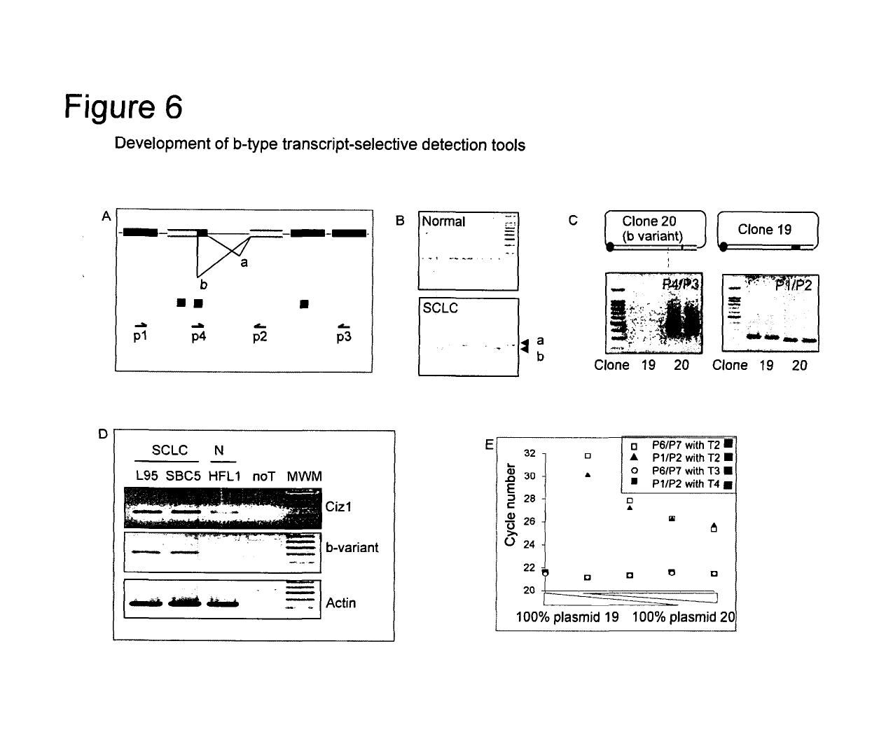

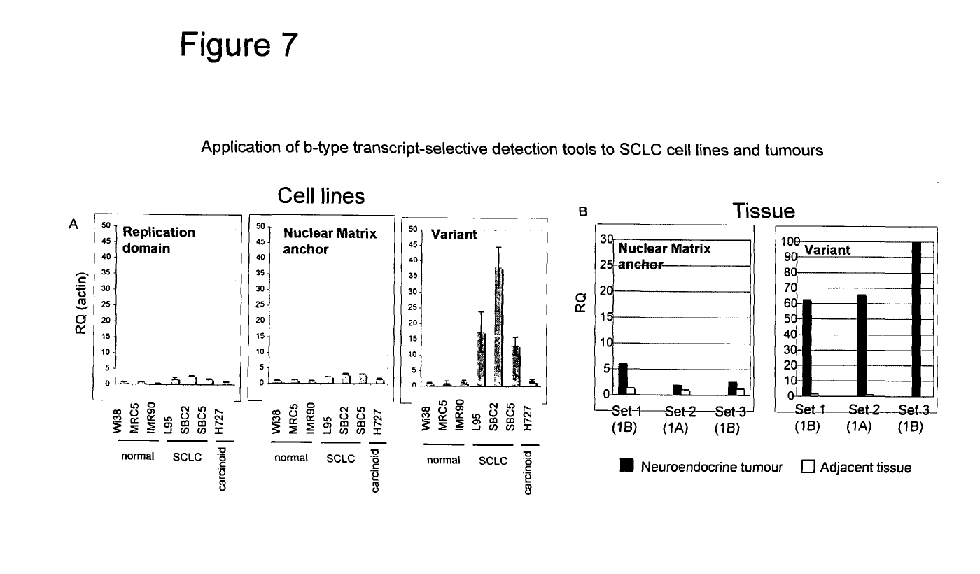

Application of selective transcript detection tools showed that cell lines derived from SCLC patients express b-type variant more often than the control cell lines (FIG. 6, 7A). Application to RNA samples derived from a small sampling of tumours from neuroendocrine lung cancer patients and also from normal adjacent lung tissue from the same patient confirms that b-type transcripts are preferentially expressed in all three SCLC patients (FIG. 7B).

B-Variant Expression in Non-Small Cell Lung Cancer

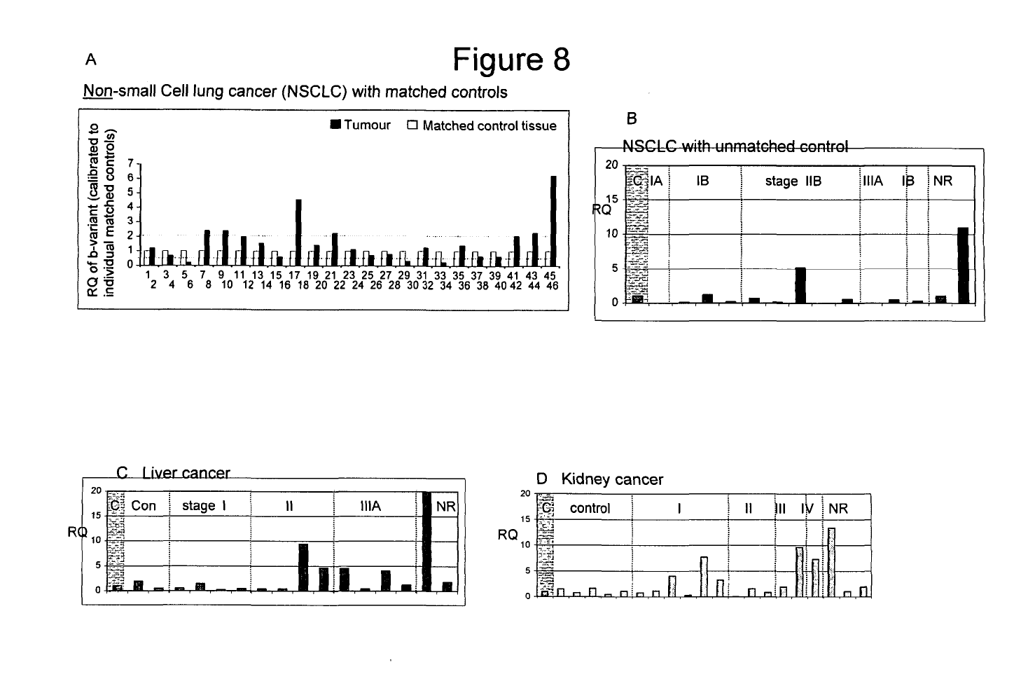

QPCR reagents that are selective for b-type transcripts were applied to the matched lung tumour/normal tissue cDNA arrays used in FIG. 1. Six of the sample sets expressed greater than 2 fold more b-type transcript in the tumour compared to normal adjacent control tissue (FIG. 8A). This includes the single neuroendocrine tumour on the array (set 9/10). Similarly, within a separate set of NSCLC samples, b-variant was elevated in a small subset of cases compared to unmatched controls (FIG. 8B). Thus expression of b-type transcripts, although prevalent in neuroendocrine tumours, is not limited to this type of lung cancer.

B-variant expression in other types of cancer

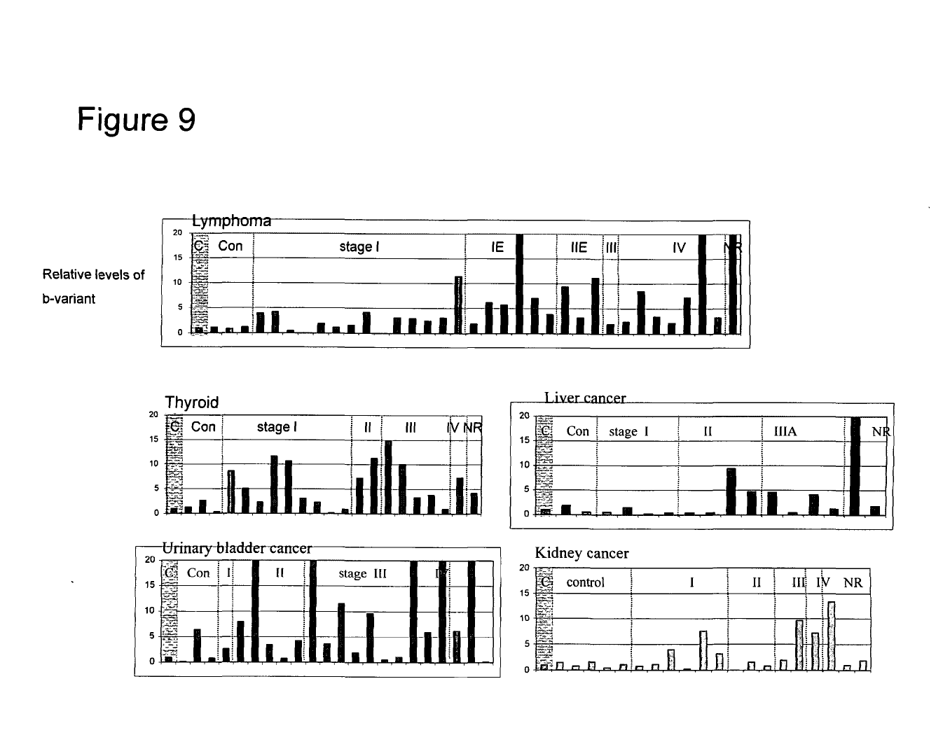

Applicant Surveyed a Range of Other Common cancers using similar cDNA arrays (Origene), that include tumours of different grade plus a set of unmatched samples from apparently normal tissue. When compared to controls, elevated b-variant was detected in a subset of liver tumours (FIG. 8C) and kidney tumours (FIG. 8D). In contrast both thyroid tumours and lymphomas express high levels of b-variant in a high proportion of cases (FIG. 9). Therefore these two tumour types are strong alternative indications for the application of Ciz1 b-variant selective diagnostic and therapeutic tools.

Ciz1 Variant Protein

High affinity variant-specific polyclonal antibodies have been generated and validated using recombinant proteins (FIGS. 10A, 10B, and 10C) and endogenous b-variant protein in SCLC cell lines (FIG. 10D). This shows that variant transcripts are indeed translated into variant protein in lung cancer cells, and that our tools are capable of effective and selective detection in a cellular context. Cizzle is also engaged in production and validation of monoclonal antibodies with the same high degree of specificity.

### Example 4

Depletion of Ciz1 from cultured mouse cells using RNA interference, inhibits progression through the cell cycle and restrains cell proliferation³. Therefore, agents that inhibit Ciz1 have potential as therapeutic molecules that restrain proliferation of cancer cells. Applicant has generated and tested human specific RNA interference molecules that inhibit Ciz1 expression, either by targeting Ciz1 generally, or by selectively targeting lung cancer-associated b-type transcripts. Both suppress proliferation of neuroendocrine lung cancer cells.

B-Type Transcript Suppression

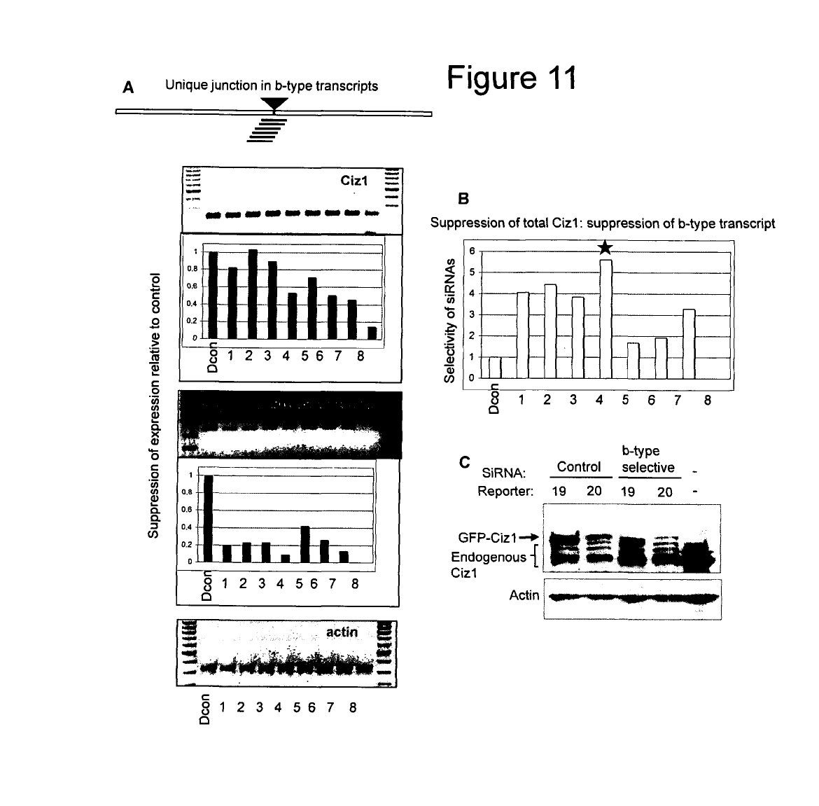

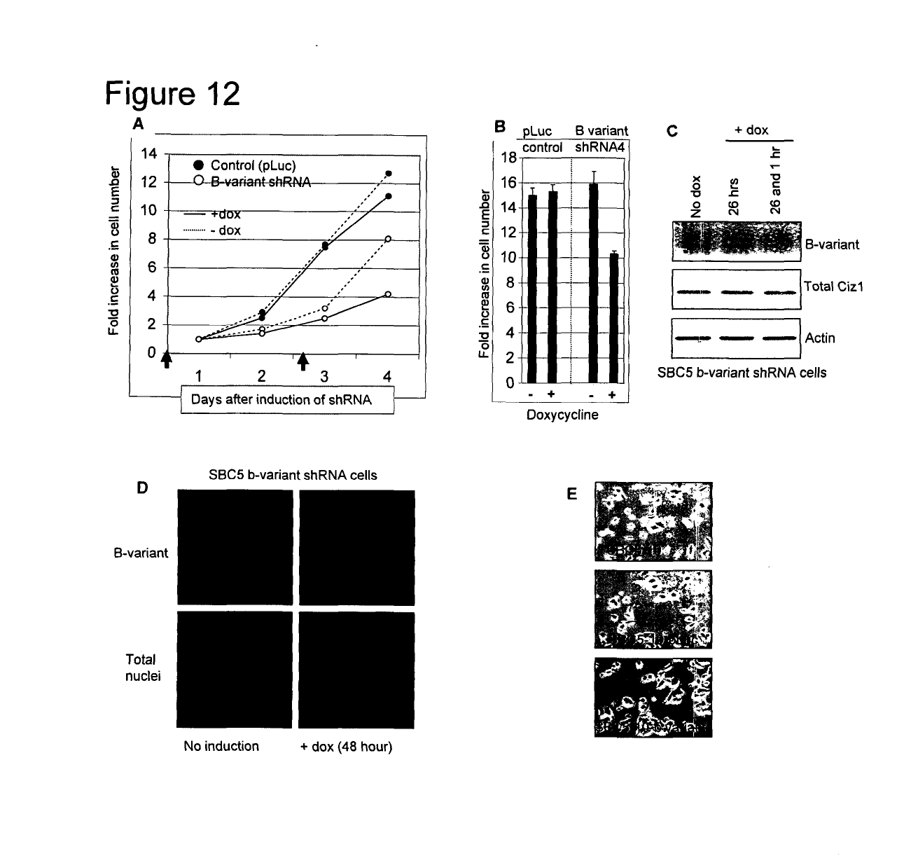

Our main strategy is to suppress b-type transcripts in a selective way with the aim of selectively suppressing the growth of lung cancer cells that express it. Candidate b-type transcript specific RNA interference molecules were compared for their ability to suppress b-type transcript expression, while leaving other forms of Ciz1 unaffected. The most effective and selective siRNA sequences were further tested for selective suppression of Ciz1 protein (FIG. 11). After transfer to an inducible shRNA delivery vector a marked effect on proliferation of SCLC cells that express endogenous b-type transcripts was recorded (FIG. 12A), along with selective suppression of b-variant transcript (FIG. 12B) and protein (FIG. 12C). Over a 4 day time course growth was suppressed to approximately 35% of similarly treated control cells (FIG. 12D). During prolonged culture with b-variant suppression notable changes in cellular morphology were observed (FIG. 12E).

Target Suppression In Vivo

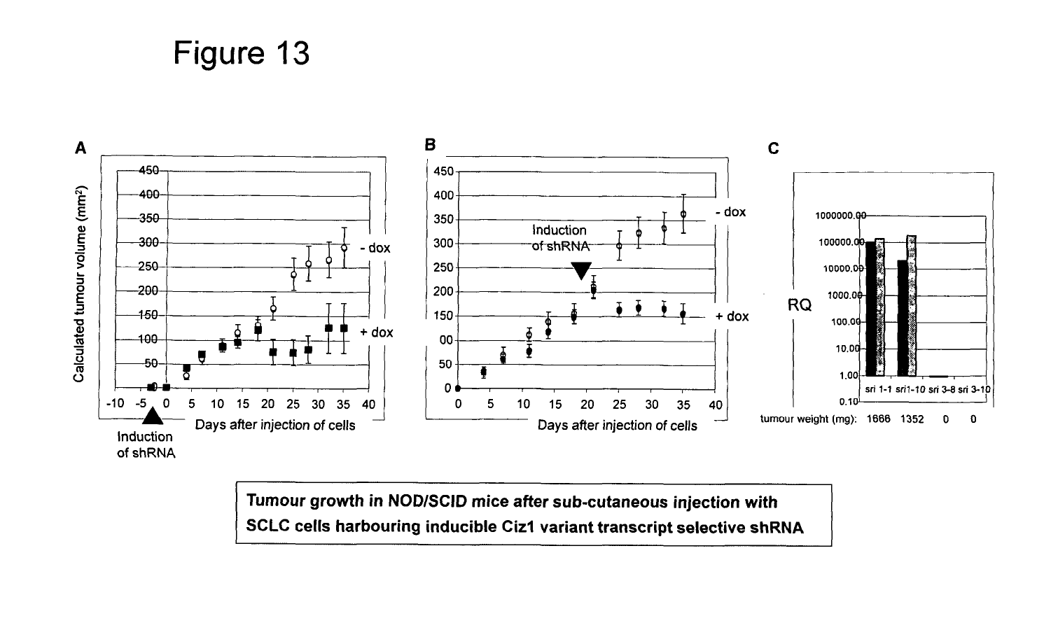

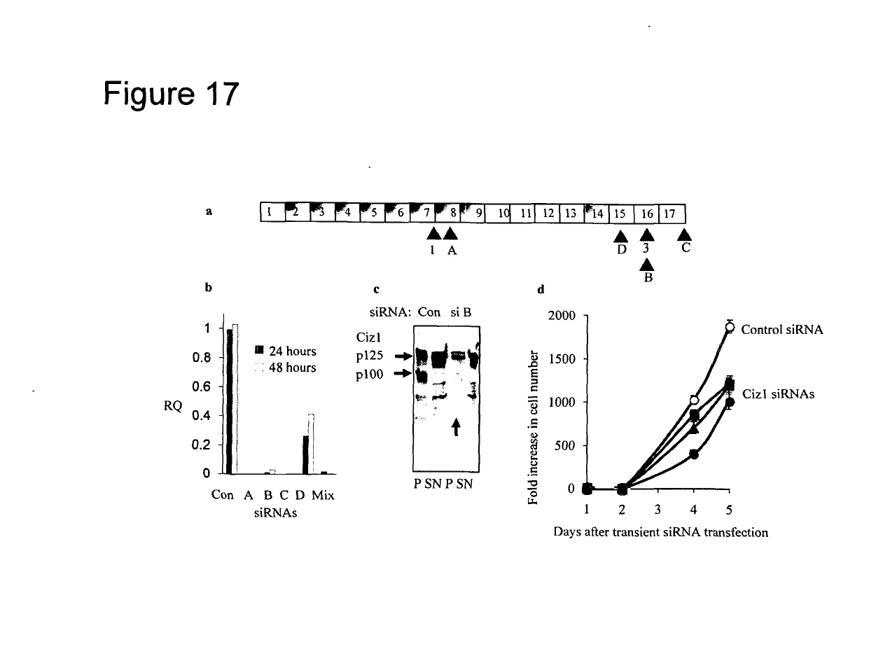

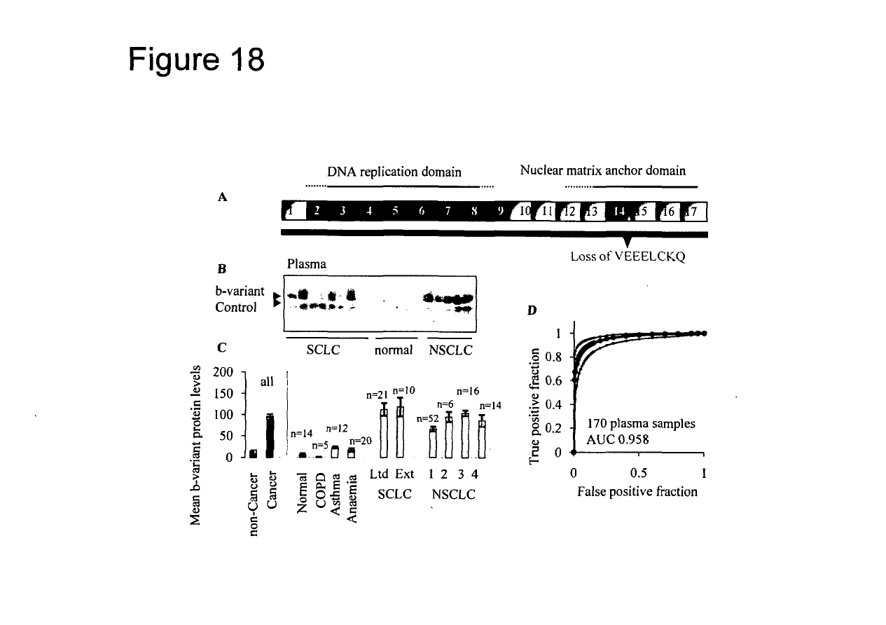

The same SCLC cells harbouring an inducible shRNA delivery vector were used to produce tumours in mice by sub-cutaneous injection. Whether activated from the date of cell injection, or switched on after tumours had formed, b-type transcript-selective RNAi effectively inhibited tumour growth in vivo (FIG. 13A, B). These data indicate that targeting the SCLC-associated Ciz1 splice variant (b-type transcript) is a potentially viable strategy for selective suppression of cell proliferation in tumour types that express it. Additional validation is planned to encompass a lymphoma-based model and systemic delivery of stabilized siRNA.

Detection of Circulating Tumour Cells

RNA isolated from whole peripheral blood of a subset of mice bearing subcutaneous tumours was used to test the sensitivity of b-type transcript detection tools (FIG. 13C). B-variant was easily detected in both the mice with tumours but not in both mice from the control group, raising the possibility that b-variant could form the basis of a blood test for SCLC.

1. A method of diagnosing cancer in a subject, said method comprising the steps of:
i) providing an isolated biological sample to be tested;
ii) detecting whether a Ciz1 b-variant polypeptide is present in said sample,
wherein the presence of said Ciz1 b-variant polypeptide indicates said subject has cancer.

2. The method of claim 1, wherein said cancer is selected from lung, lymphoma, kidney, breast, liver, bladder and thyroid cancer.

3.-6. (canceled)

7. The method of 6 claim 1, wherein said cancer is non-small cell lung cancer (NSCLC).

8. The method of claim 2, wherein said lung cancer is small cell lung cancer (SCLC).

9.-17. (canceled)

18. The method of claim 1, wherein said method comprises the step of imaging the subject's lungs.

19. The method of claim 18, wherein said imaging further comprises the step of performing a chest X-ray, computerized tomography (CT) scan, magnetic resonance imaging (MRI) scan or positron emission tomography (PET) scan, and wherein said imaging alone is insufficient for said diagnosing of cancer.

20.-25. (canceled)

26. The method of claim 1, wherein said Ciz1 b-variant polypeptide comprises the amino acid sequence DEEEIEVRSRDIS (SEQ ID NO: 8).

27. The method of claim 26, wherein said Ciz1 b-variant polypeptide comprises the amino acid sequence of SEQ ID NO: 22.

28. The method of claim 1, wherein said biological sample is tissue, blood, plasma, sputum, bronchoalveolar lavage, bronchoalveolar brushing or urine.

29.-36. (canceled)

37. The method of claim 1, wherein said Ciz1 b-variant polypeptide is extracellular.

38.-48. (canceled)

49. The method of claim 1, wherein said method further comprises the step of contacting said biological sample with a Ciz1 b-variant polypeptide binding agent.

50. The method of claim 49, wherein said Ciz1 b-variant polypeptide binding agent is an antibody or antigen binding fragment thereof.

51.-56. (canceled)

57. The method of claim 49, wherein said Ciz1 b-variant polypeptide binding agent specifically binds a Ciz1 b-variant polypeptide comprising the amino acid sequence SEQ ID NO: 22.

58. The method of claim 57, wherein said Ciz1 b-variant polypeptide binding agent specifically binds a Ciz1 b-variant polypeptide comprising the amino acid sequence of SEQ ID NO: 8.

59.-63. (canceled)

64. The method of claim 1, wherein said method further comprises the step contacting said biological sample with a second Ciz1 b-variant polypeptide binding agent, wherein said second Ciz1 b-variant polypeptide binding agent recognizes an epitope other than an epitope spanning exons 14b and 15.

65.-79. (canceled)

80. An isolated Ciz1 b-variant polypeptide binding agent that specifically binds a Ciz1 b-variant polypeptide.

81. The binding agent of claim 80, wherein said Ciz1 b-variant polypeptide binding agent specifically binds a Ciz1 b-variant polypeptide comprising the amino acid sequence SEQ ID NO: 22.

82. The binding agent of claim 81, wherein said Ciz1 b-variant polypeptide binding agent specifically binds a Ciz1 b-variant polypeptide comprising the amino acid sequence of SEQ ID NO: 8.

83.-105. (canceled)

106. A kit for diagnosis and prognosis of cancer in a subject comprising a component for detecting the presence of a Ciz1 polypeptide in a biological sample.

107. The kit of claim 106, wherein said component for detecting the presence of a Ciz1 polypeptide is a Ciz1 binding agent.

108.-121. (canceled)
